(19) United States
(12) Patent Application Publication
(10) Pub. No.: US 2026/0146254 A1
(43) Pub. Date: May 28, 2026

(54) Enhancing Oligonucleotide Immunomodulatory Activity through Dianophore Long-Lasting Modification: Methods and Applications

(71) Applicants: Susanna BOSI, Trieste (IT); Sergio Gabriel TISMINETZKY, Trieste (IT); Segena Corporation S.A., Montevideo (UY)

(72) Inventors: Susanna Bosi, Trieste (IT); Sergio Gabriel Tisminetzky, Trieste (IT)

(73) Assignee: Segena Corporation S.A., Montevideo (UY)

(21) Appl. No.: 19/121,211

(22) PCT Filed: Nov. 20, 2023
(86) PCT No.: PCT/IB2023/061699

§ 371 Date: Apr. 15, 2025

Related U.S. Application Data
(60) Provisional application No. 63/426,808, filed on Nov. 21, 2022.

(51) Int. Cl. C12N 15/117; A61P 31/02; A61P 35/00; A61P 37/06

(52) U.S. Cl. CPC C12N 15/117; A61P 31/02; A61P 35/00; A61P 37/06; C12N 2310/17; C12N 2310/315; C12N 2310/351; C12N 2310/3515; C12N 2330/30

(57) ABSTRACT

Dianophore Long-Lasting Modifications of oligonucleotides with immunomodulatory and homeostatic properties (including the proliferation of pluripotent mesenchymal stem cells (MSC) of animals, including humans). Different ONs were synthetized with a modification at their backbone structure and chemically combined with some specific organic molecules, modifying the original dianophore (but not the pharmacophore) of the original ON, yielding several long-lasting conjugates. The obtained conjugates are not susceptible to in vivo hydrolytic cleavage and have enhanced in vivo half-life, also in its natural phosphodiester form and have shown much more biological potency (hundred to thousand times). These modified oligonucleotides are used in a wide range of clinical treatments involving the immune system and/or regeneration of mesenchymal tissues which have been damaged through acute injury, abnormal genetic expression or acquired disease. In addition, relates to the impact of LLONC (Long-lasting Oligonucleotide Conjugates) inoculation, having biologically active oligonucleotides (phosphodiester or partially/fully phosphorothioated) with various dianophore modifications.

### BACKGROUND

The first oligonucleotide (ON) technology explored for therapeutic purposes was based on antisense technology, which relies on single-stranded sequences of nucleotides that are complementary to RNA transcripts in human cells. Antisense gapmer, steric block, spliceswitching ONs, and short interfering RNA drugs have been successfully developed. Moreover, antagomirs, microRNA mimics, aptamers, DNA decoys, DNAzymes, synthetic guide strands for CRISPR/Cas, and innate immunity-stimulating ONs are all undergoing clinical trials. Also, DNA targeting, triplex-forming ONs, strand-invading ONs, CpG ON and PyNTTTTGT ONs, have made their mark on drug development research, but not yet as medicines.

In all cases, the design of synthetic nucleic acid chemistry is crucial for achieving biologically active immunomodulatory oligonucleotides IM-ONs. The main restriction for therapeutic ONs is their pharmacokinetics: in contrast to lipophilic small molecules, which are readily taken up by cells, the majority of the ON medicines are more hydrophilic and larger in size, making their uptake challenging.

In most cases, after systemic administration, ON are exposed in the bloodstream to a rapid degradation by nucleases and fast clearance via the reticuloendothelial system (RES) and renal filtration. Then, in most cases, for expressing their activity ONs need to cross the vascular endothelial barrier and diffuse through the extracellular matrix to approach the target cells in tissue and, finally, cross the plasma membrane and the endosomal membrane to access their gene targets in the cytoplasm and/or nucleus of cells.

In the case of CpG immunomodulatory ONs and PyNTTTTGT homeostatic ONs, the path is simpler because they only have to reach to the surface receptors of some blood cells (plasmacytoid dendritic cells (pDCs) and B cells in case of CpG ONs and just B cells in case of PyNTTTTGT ONs) to trigger their biological activity. But, for reaching their target both should remain in blood circulation as much as possible where are especially exposed to nucleases degradation and clearance via RES and renal filtration.

Most of them (antisense gapmer, spliceswitching ONs, short interfering RNA, antimicroRNAs, microRNA mimics, aptamers, DNA decoys, DNAzymes, guide strands for CRISPR/Cas) should have a whole specific sequence, but CpG ONs (i) and PyNTTTTGT ONs (ii) constitute ON families whose members just have to have one or more active sites with a characteristic sequence (CpG or PyNTTTTGT, respectively), immerse in almost any aleatory ON sequence for keeping their biological activity. FIG. 1 shows the most representative molecules of each group in its fully phosphorotioated form, CpG2006 and IMT504, respectively.

By optimizing synthetic oligonucleotide (ON) chemical modification strategies, ON pharmacokinetics and pharmacodynamics properties have become profoundly enhanced. Only in recent years, when ONs have become sufficiently potent, it has been possible to study how to convert them to drug-like molecules.

The most common modifications are phosphorothioate linkages, base methylation, and numerous 2-substitutions in the furanose ring, such as 2-fluoro, O-methyl, or methoxyethyl. Locked nucleic acid and constrained ethyl, a related variant, are bridged forms where the 2-oxygen connects to the 4-carbon in the sugar. Phosphorodiamidate morpholino-oligomers, carrying a modified heterocyclic backbone ring, have also been developed.

However, most of ONs have still a short pharmacological half-life and, consequently, it can be difficult to achieve therapeutically useful blood levels of the ON in patients.

CpG-containing oligodeoxynucleotides (ODNs) stimulate cells that express Toll-like receptor 9 (TLR9), inducing inflammatory cytokines and type I interferon (IFN) to activate humoral or cellular immunity.

More than 600 preclinical studies investigating the treatment or prevention of cancers, infections, and allergies with CpG ONs have been implemented to date (iii), and more than 100 clinical trials have either been completed or are currently in progress (iv). Many of the CpG ONs developed to date have been single-stranded synthetic ONs with ˜20 bases, and they are divided into four classes depending on the differences in their structure and immunoreactivity. Among these four classes, almost all the CpG ONs used in clinical trials have been class-B CpG (CpG-B) ONs (also known as K-type ONs), whereas class-A CpG (CpG-A) ONs (also known as D-type ONs) have also been used but in fewer clinical trials. All the nucleotides of CpG-B ONs used on these trials are phosphorothioated and contain one or more nonpalindromic CpGs in the basic sequence (v, vi, vii, viii); usually, several consecutive injections of high doses of the fully phosphorotioated ON have to be administered in order to obtain acceptable therapeutic results, sometimes, close to the toxic dose.

The most representative molecule of this group is the ON called CpG2006 (known in pharma as CpG7909), with a formula:

CpG 2006:
(SEQ ID No: 1)
5′-TCGTCGTTTTGTCGTTTTGTCGT-3′

On the other hand, PyNTTTTGT ONs, a family of homeostatic oligonucleotides, induce immunomodulation, but also “in vivo” expansion of MSC resulting in wider range of therapeutic applications compared with CpG IM-ONs. These homeostatic ONs have shown a marked improvement of animals suffering nociceptive, neuropathic, and “other” (Nocipathic or Nociplastic) pain, immune suppression, osteoporosis, diabetes, multi-organ failure and sepsis (ix). However, 5 daily and consecutive injections of high doses of the fully phosphorotioated ON have to be administered in order to obtain acceptable therapeutic results.

The most representative molecule of this group is the ON called IMT504, whose formula is:

IMT504:
(SEQ ID No: 2)
5′-TCATCATTTTGTCATTTTGTCATT-3′

Then, one of the non-minor problem during clinical application of RNA/DNA pharmaceuticals, like all those previously mentioned, is their degradation by DNases.

Due to this, nucleotides of CpG-A ONs are partially phosphorothioated, and all the nucleotides of CpG-B ONs are fully phosphorothioated. The half-life of phosphorothioated DNA in vivo is 30-60 minutes, which is a longer lifespan than the half-life of non-phosphorothioated natural phosphodiester DNA [5-10 minutes] (x), but this is still not an adequate length of time.

Similarly, the use of a phosphorothioated backbone in homeostatic PyNTTTTGT ONs is convenient because of its relative nuclease resistance, but it is not an absolute requirement, since as phosphodiester PyNTTTTGT ONs are also active. Despite this, a higher concentration and iterative administrations of phosphodiester ONs should be used (around 50 times more) in order to obtain the same biological effect induced by the corresponding phosphorothioated ONs (xi, 1).

Several associated adverse drug reactions have been described (xii, xiii, xiv, xv, xvi, xvii, xviii) that represented a concern for the use of phosphorothioated CpG ONs. There is much less information about PyNTTTTGT ONs, but they also appeared to be toxic at higher dose (77).

Toxicity was also described for all phosphorothioated antisense oligonucleotides (^{19, 20}).

In summary, when we extrapolate the aforementioned findings to the pharmaceutical industry, several unresolved challenges must be addressed to fully harness the therapeutic potential of oligonucleotides. Firstly, the current capacity for manufacturing therapeutic ONs remains limited, imposing a significant constraint in terms of both cost and accessibility, constituting an important industrial limitation. Then, developing more powerful NOs that allow therapeutic doses to be drastically reduced becomes a critical issue. Work is also being done to increase the half-life in circulation, trying to reduce the number of inoculations necessary to achieve the desired therapeutic effect. Finally, many of the current developments (even approved products) present very high levels of toxicity; In many cases this toxicity is due to the chemical modifications carried out on the NOs, seeking to increase their half-life in circulation; The same fact that makes its degradation by nucleases more difficult makes its metabolic degradation through normal pathways impossible, resulting in toxic effects on the organs involved in its purification. Therefore, it becomes very important to find a solution to this problem in a different way.

In the case of traditional small molecule drugs, their therapeutic properties are inseparable from their pharmacodynamic, pharmacokinetic, and absorption, distribution, metabolism, and excretion (ADME) behavior. Thus, improving therapeutic properties requires a unique and iterative optimization process for each different molecule.

Oligonucleotides (ONs), which are short DNA or RNA sequences typically consisting of 10-40 nucleotides, represent a novel category of therapeutic agents known as “informational” therapeutics [^{21, 22, 23}]. Unlike traditional small-molecule drugs (where changes in chemical structure usually result in alterations of both, biological and pharmacokinetic properties), ON-based drugs exhibit a pharmacokinetic behavior, including absorption, distribution, metabolism, and excretion (ADME), as well as toxicity, that are largely independent of the specific information encoded within their base sequences, linked to the therapeutic activity. Consequently, once an optimized formulation for efficient and safe delivery of ONs is developed, various disease targets can be addressed simply by modifying the ON sequences. This inherent advantage over small-molecule drugs, coupled with the rapid progress in understanding disease mechanisms at the molecular level and advancements in gene sequencing, holds the potential for significantly accelerated drug development cycles, reduced costs, and the prospect of personalized treatments tailored to specific needs. [^{24, 25, 26, 27}].

The informative nature of oligonucleotide drugs, which are drugs designed based on sequence information, promised to lend themselves well to the post-genomic era of medicine [xix]. Researchers were attracted to the promise of rapid and rational drug design against virtually any sequence-dependent target. As with any therapeutic modality, the success of an oligonucleotide drug is defined by both its ability to affect a target and its pharmacokinetic behavior, including ADME, mostly related also with the molecule toxicity.

Oligonucleotide treatments comprise a diverse class of drugs that act by different therapeutic mechanisms but are all guided by a specific sequence of nucleotide bases. However, the intensity of the activity and the pharmacokinetic properties can be optimized to some extent independently [²⁹].

The pharmacokinetic properties of a drug depend on a set of molecular characteristics that we refer to as a dianophore, from the Greek word “dianomi” which means distribution or delivery. For oligonucleotide drugs, the dianophore is largely defined by chemical and structural architecture, such as chemical modifications of the sugars, bases, and phosphate or phosphorothioate backbone, single-stranded or duplex structure, and the presence or absence of a ligand. On the contrary, the pharmacophore (the set of molecular characteristics that determine the biological effect resulting in a therapeutic activity) is defined by its nucleotide sequence. Although the sequence of bases and the precise pattern of chemical modifications can affect in certain degree the overall properties of an oligonucleotide and its trafficking, cellular uptake, and other behaviors, the ability to optimize the pharmacophore and dianophore separately is a key advantage of oligonucleotide drugs.

But, despite extensive research, the development of ON-based drugs has not progressed as rapidly as initially anticipated, resulting in only sixteen drugs reaching the market in the past two decades [^{30, 31}]. This slow bench-to-bedside translation is attributed to the inherent challenges associated with ON drugs [^{32, 33}]. Unmodified, “naked” ONs are susceptible to degradation by nucleases, quickly cleared by renal and hepatic pathways, and face significant barriers to cellular entry due to their hydrophilicity and high molecular weight [^{34, 35}]. To overcome these challenges, advanced delivery systems such as polycationic polymers, nanoparticles, or liposomal formulations [^{36, 37, 38, 39}], as well as direct chemical modifications of the ONs [⁴⁰], have been explored.

However, aside from liposomes, most carrier systems still require clinical validation [⁴¹]. The key challenges include concerns related to toxicity, immunogenicity, formulation consistency (especially for complex systems with multiple components), chemical and in vivo stability, controlled release, and large-scale manufacturing. Among the direct chemical modifications, the most commonly used approach is the phosphorothioate backbone [⁴²], which enhances ON stability against enzymatic degradation and improves cellular uptake [⁴³]. Additionally, phosphorothioates enable ONs to associate with plasma proteins, thereby preventing rapid renal clearance [⁴⁴]. Although other modifications, primarily 2′-modifications and conformation prearrangements, can enhance stability and binding affinity, their effectiveness often relies on their combination with phosphorothioates [⁴⁵].

It is important to note that chemical modifications have their limitations. For instance, phosphorothioates can reduce the binding affinity of ONs for their targets, sometimes requiring additional modifications to compensate [⁴⁶]. Moreover, phosphorothioates are associated with nonspecific adverse effects, including the induction of stress responses, prolonged activated partial thromboplastin time (aPTT), thrombocytopenia, and increased serum transaminase activities [^{47, 48, 49}]. Consequently, there is a continuous search for new modifications or strategies that can replace or reduce the reliance on phosphorothioates.

FIG. 2 shows the pharmacophore structure of CpG ONs (A) and PyNTTTTGT ONs (B) where they stand out, in larger size, the positions of the active groups in both types of ON. It is also shown the dianophore structure (basically the backbone) of a 24 mer ODN responsible of their pharmacokinetic behavior, including ADME, which is mostly related to the molecule toxicity (C).

The development of an optimized dianophore, a chemical architecture that allows effective delivery of an effector to a given tissue and a predictable ADME profile, would allow to build easily new drugs for multiple indications, according to the chosen pharmacophore. Initially, unmodified or minimally modified compounds were rushed to the clinic without delivery vehicles. The massive dose requirements and limited clinical efficacy created a dramatically negative view of the technology, damaging the reputation of the field of oligonucleotide therapy for years, taking more than three decades to reach clinical maturity. Advances in oligonucleotide chemistry and understanding of the fundamental principles that define the in vivo behavior of oligonucleotides have allowed ODN therapies to approach clinical productivity. As a result, the current portfolio of oligonucleotide drugs is broad and includes a variety of molecules with different mechanisms of action.

However, the concept of conserving the pharmacophore sequences suggests that the properties responsible for its therapeutic activity would be maintained. By targeting the improvement of the dianophore, the general pharmacologic properties of the drug can be enhanced while keeping the therapeutic objective unchanged.

Having this in mind, both problems, stability against nucleases and toxicity, may be overcome by conjugating the ONs to an adequate ligand without altering the pharmacophore structure. Additionally, using some specific ligands able to extend the half-life in blood circulation by means of slowing down the clearance via RES and renal filtration, would avoid the need of repetitive and frequent injections. Then, we would be able to conserve all pharmacological activities, but with better performance as a medicine. If the original ON is useful for treating a disease or health condition, the improved molecules with a modified dianophore will also be.

There are numerous examples where the conjugation of therapeutics with polymers, natural or artificial, can prolong the residence times of aptamers in circulation, reducing the frequency of dosing and producing an improvement in efficacy to achieve different therapeutic objectives.

The industrial process for manufacturing large-scale of long-lasting oligonucleotide compounds (LLONCs) that meet the standards set by most of international regulatory agencies, involves significant dedication of time and money. Then, there is a need to optimize efficient and feasible manufacturing methods for this class of therapeutic agents.

In some embodiments, the ligands are coupled, preferably covalently to a ligand. In exemplary embodiments, the ligand is directly linked to the carrier or through an intermediate spacer. In exemplary embodiments, a ligand alters the distribution, targeting ability or lifespan of the oligonucleotide to which it is bonded. In some embodiments, a ligand provides improved affinity for a selected target, for example, molecule, cell or cell type, for a selected compartment, for example, a cell compartment or organ, tissue, organ or region of the body, in comparison with the same oligonucleotide without said ligand. In exemplary embodiments, a ligand provides improved biological activity, in comparison with the same oligonucleotide without said ligand.

Exemplary ligands can improve transport, receptor affinity and specificity and may also improve nuclease resistance of the resultant conjugate oligonucleotide. General examples of ligands include lipophiles, lipids, steroids (e.g., uvaol, hecigenin, diosgenin), terpenes (e.g., triterpenes, e.g., sarsasapogenin, friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics. Ligands can include a naturally occurring substance, (e.g., human serum albumin (HSA), low-density lipoprotein (LDL), or globulin); carbohydrate (e.g., a dextran, pullulan, chitin, chitosan, inulin, cyclodextrin or hyaluronic acid); amino acid, or a lipid. The ligands may also be natural, recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl) methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine, N-acetyl-glucosamine, multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. Other examples of ligands include dyes, intercalating agents (e.g. acridines and substituted acridines), cross-linkers (e.g. psoralene, mitomycin C), porphyrins (TPPC4, texaphyrin, Sapphyrin), polycyclic aromatic hydrocarbons (e.g., phenazine, dihydrophenazine, phenanthroline, pyrenes), Lys-Tyr-Lys tripeptide, aminoglycosides, guanidium aminoglycodies, artificial endonucleases (e.g. EDTA), lipophilic molecules, e.g, cholesterol (and thio analogs thereof), cholic acid, cholanic acid, lithocholic acid, adamantane acetic acid, 1-pyrene butyric acid, dihydrotestosterone, glycerol (e.g., esters (e.g., mono, bis, or tris fatty acid esters, e.g., C10, C11, C12, C13, C14, C15, C16, C17, C18, C19, or C20 fatty acids) and ethers thereof, e.g., C10, C11, C12, C13, C14, C15, C16, C17, C18, C19, or C20 alkyl; e.g., 1,3-bis-O (hexadecyl)glycerol, 1,3-bis-O (octaadecyl)glycerol), geranyloxyhexyl group, hexadecylglycerol, borneol, menthol, 1,3-propanediol, heptadecyl group, palmitic acid, stearic acid (e.g., glyceryl distearate), oleic acid, myristic acid, O3-(oleoyl) lithocholic acid, O3-(oleoyl) cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, naproxen, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the oligonucleotide into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The ligand can increase the uptake of the oligonucleotide into the cell by activating an inflammatory response, for example. Exemplary ligands that would have such an effect include tumor necrosis factor alpha (TNFα), interleukin-1 beta, or gamma interferon. In one aspect, the ligand is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA. A lipid-based ligand can be used to modulate, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney. In a preferred embodiment, the lipid-based ligand binds HSA. A lipid-based ligand can bind HSA with a sufficient affinity such that the conjugate will be preferably distributed to a non-kidney tissue.

However, it is preferred that the affinity not be so strong that the HSA-ligand binding cannot be reversed. In another preferred embodiment, the lipid-based ligand binds HSA weakly or not at all, such that the conjugate will be preferably distributed to the kidney. Other moieties that target to kidney cells can also be used in place of or in addition to the lipid-based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low-density lipoprotein (LDL).

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the Oligonucleotide, such as by enhancing cellular recognition and absorption. The peptide or peptidomimetic moiety can be about 5-50 amino acids long, e.g., about 5, 10, 15, 20, 25, 30, 35, 40, 45, or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. The peptide moiety can be an L-peptide or D-peptide. In another alternative, the peptide moiety can include a hydrophobic membrane translocation sequence (MTS). The peptide moieties can have a structural modification, such as to increase stability or direct conformational properties. Any of the structural modifications described below can be utilized. These problems may be overcome by conjugating the ONs to polymers such as polyethylene glycol or Hyaluronic acid or even fatty acids as we shown in the present patent.

In order to elude (if required) the need of a phosphorothioate backbone in therapeutic ONs due to its nuclease resistance, and to facilitate the drug administration (reducing several injections, each every day or week—as therapies appears to need—to only one shot) for reaching maximal activity, conjugation of the ONs with polymers such as Hyaluronic acid or polyethylene glycol or even fatty acids were tested.

In some embodiments, the ligands were coupled, preferably covalently, directly through an intermediate spacer, to a ligand. In some embodiments, both types of ODNs, CpG and PyNTTTTGT were coupled with different spacers (FIG. 3), yielding a molecule with a reactive amino group, preferably at 3′ end, getting different variants, like, for example, phosohodiester or phosphotioate/C3 spacer ODNs showed in FIG. 4.

### Hyaluronic Acid Conjugates

Hyaluronic acid (HA) is a high molecular weight glycosaminoglycan polymer composed of repeating disaccharides: β1,3 N-acetyl glucosaminyl-β1,4 glucuronide (FIG. 5). HA is ubiquitous, being the main component of extracellular matrix, and is essential for proper cell growth, structural stability of organs, and tissue organization.

From the pharmaceutical standpoint, HA is a promising component, because it is biodegradable, biocompatible, nontoxic, hydrophilic, and non-immunogenic. HA contains several chemical groups to which other components can be conjugated (FIG. 5).

In the most of the cases, active molecules are covalently linked at the carboxylic moiety of the glucuronic acid subunit. Alternatively, the covalent link can occur on the C-6 position of the N-acetyl-D-glucosamine residue of the hyaluronic acid (xx, xxi and xxii).

Other inventive drug delivery systems (DDS) are described containing hyaluronic acid and a therapeutic agent, wherein the therapeutic agent is linked, directly or via a linker, to 6-aminohyaluronic acid and where the linkage of the drug or linker with 6-aminohyaluronic acid consists in an amide bond (xxiii and xxiv). In addition, in this case the 6-position is involved because the OH is substituted with an amino group NH2. This group is then amidated with a COOH belonging to a spacer or to the molecule that is linked. Preferred therapeutic agents for use in the present DDS are anti-inflammatory, antibiotic, antitumor drugs. Preferred linkers are: succinic acid, succinic acid linked to aminoacids, succinic acid linked to peptides. Other inventive drug delivery systems (DDS) are described containing hyaluronic acid and a therapeutic agent, wherein the therapeutic agent is linked, directly or via a linker, to 6-aminohyaluronic acid and where the linkage of the drug or linker with 6-aminohyaluronic acid is realized by an amide bond. In addition, in this case the 6-position is involved because the OH is substituted with an amino group NH2. This group is then amidated with a COOH belonging to a spacer or to the molecule that is linked. Preferred therapeutic agents for use in the present DDS are anti-inflammatory, antibiotic, antitumor drugs. Preferred linkers are: succinic acid, succinic acid linked to aminoacids, succinic acid linked to peptides. The DDS are stable and free of undesired reaction by-products and impurities, and show a high level of pharmacological efficacy.

Alternatively, polysaccharide esters of N-derivatives of glutamic acid (N-GA derivatives) have been used (xxv). These polysaccharidic esters have antiproliferative activity and are characterized by a low systemic toxicity. The esters of the invention are used in the prevention and therapy of diseases caused by cellular hyperproliferation, particularly psoriasis, tumors, rheumatoid arthritis, or intestinal inflammatory pathologies. Derivatization of all the possible polysaccharides with N-derivatives of glutamic acid was described.

Conjugates containing camptothecin connected to hyaluronic acid via a linker were also disclosed, where the linker is bound to the hyaluronic acid through an ester bond (xxvi). Also this patent, related to the specific conjugate HA-CPT, involves the C6 on the glucosamine unit position on the hyaluronic acid. The ester bond involves on one side, a hydroxyl group of hyaluronic acid and, on the other side, a carboxyl group present on the linker; the linker is covalently bonded to camptothecin. Pharmaceutical compositions thereof, their use in the treatment of pathologies responsive to camptothecin, and a process to prepare said conjugates are also described.

### Polyethylene Glycol Conjugates

Poly(ethylene glycol) (PEG), which is the current gold standard for stealth polymers in the emerging field of polymer-based drug delivery, is in competition with other polymers that have convenient features such as biodegradability. PEG still holds first place as far as PEGylated derivatives under investigation in clinical trials are concerned, the development of new copolymers based on PEG or PEG derivatives are trying to overcome some of its drawbacks as for example the hurdle of biodegradability.

PEG appears to be one of the most important and widely used polymer in the field of drug delivery. PEG's high biocompatibility, low toxicity, and shielding effect are properties that have already been described in several reviews (xxvii; xxviii and xxix). Clinically used PEG conjugates accounts for 15 approved products as, for example PegIntron, Pegasys, Oncaspar, Somavert . . . The numerous studies on PEGylation described in the literature constitute a valuable reference and evidence of its many advantages for researchers working on new projects. PEG polymers are commercially available with different reactive end groups and several molecular weights and architectures (i.e. linear, mono- or bi-functional, heterobifunctional, branched, forked, multi-arm, comb-shaped, etc.) that are characterized by high purity and low polydispersity. PEG is non-biodegradable so its in vivo use requires the use of molecular weights below the kidney excretion threshold, approximately 40 kDa (xxx).

PEG is a versatile molecule that can be linked to virtually any possible domain on the selected molecule on interest. The choice of the suitable functional group for the PEG derivative is based on the type of available reactive group on the molecule that will be coupled to the PEG. For proteins, typical reactive amino acids include lysine, cysteine, histidine, arginine, aspartic acid, glutamic acid, serine, threonine and tyrosine. The N-terminal amino group and the C-terminal carboxylic acid can also be used as a site (specific site by conjugation with aldehyde functional polymers (32).

The techniques used to form first generation PEG derivatives are generally reacting the PEG polymer with a group that is reactive with hydroxyl groups, typically anhydrides, acid chlorides, chloroformates and carbonates. In the second generation PEGylation chemistry more efficient functional groups such as aldehyde, esters, amides etc. made available for conjugation. Third generation pegylation agents, where the polymer has been branched, Y shaped or comb shaped are available and show reduced viscosity and lack of organ accumulation (xxxi).

Many forms of ONs, including antisense, aptamer, siRNA, and miRNA have been PEGylated, and one PEGylated aptamer has received regulatory approval. It is recognized that PEGylation alone may not achieve the necessary biopharmaceutical improvement for ON drugs, and a combination with another chemical modification is needed (xxxii. With the popular use of phosphorothioate ONs, which show improvement in bioavailability, enzyme stability, and efficacy, PEGylation is oftentimes not carried out due to the lack of significant additional benefits. Nonetheless, phosphorothioates are imperfect, showing reduced binding affinity and nonspecific stress and toxic responses, and alternatives are still sought after.

### Lipidic Acid Conjugates

Fatty acids have been previously conjugated with drugs to help the drugs as conjugates cross the blood brain barrier. DHA (docosahexaenoic acid) is a 22 carbon naturally-occurring, unbranched fatty acid that previously has been shown to be unusually effective, when conjugated to a drug, in crossing the blood brain barrier. DHA is attached via the acid group to hydrophilic drugs and renders these drugs more hydrophobic (lipophilic-xxxiii, xxxiv-). DHA is an important constituent of the brain and recently has been approved as an additive to infant formula. The mechanism of action by which DHA helps drugs conjugated to it cross the blood brain barrier is unknown.

Another example of the conjugation of fatty acids to a drug is the attachment of pipotiazine to stearic acid, palmitic acid, enanthic acid, undecylenic acid or 2,2-dimethyl-palmitic acid. Pipotiazine is a drug that acts within the central nervous system. The purpose of conjugating pipotiazine to the fatty acids was to create an oily solution of the drug as a liquid implant for slow release of the drug when injected intramuscularly. The release of the drug appeared to depend on the particular fatty acid selected, and the drug was tested for its activity in the central nervous system (65, 66, 67, 68).

Lipidic molecules, including the fatty acids, also have been conjugated with drugs to render the conjugates more lipophilic than the drug. In general, increased lipophilicity has been suggested as a mechanism for enhancing intestinal uptake of drugs xxxv into the lymphatic system, thereby enhancing the entry of the conjugate into the brain and thereby avoiding first-pass metabolism of the conjugate in the liverxxxvi. The type of lipidic molecules employed have included phospholipids, non-naturally occurring branched and unbranched fatty acids, and naturally occurring branched and unbranched fatty acids ranging from as few as 4 carbon atoms to more than 30 carbon atoms. In one instance, enhanced receptor binding activity was observed (for an adenosine receptor agonist) 71, and it was postulated that the pendant lipid molecule interacted with the phospholipid membrane to act as a distal anchor for the receptor ligand in the membrane micro environment of the receptor. This increase in potency, however, was not observed when the same lipid derivatives of adenosine receptor antagonists were used, and generalizations thus were not made possible by those studies.

### SUMMARY

The invention discloses several ways for modifying the dianophore of oligonucleotides, enabling them to maintain their original therapeutic activity by preserving the pharmacophore. However, these modifications significantly enhance the potency of the therapeutic activity, resulting in significantly higher biological activity in vivo compared to the original oligonucleotides.

The embodiments of this invention focus on compositions and methods for creating novel and highly effective long lasting modified oligonucleotides (referred to as LLONC). These modifications improve the dianophore of the original oligonucleotides, which is responsible for their pharmacokinetic behavior, including absorption, distribution, metabolism, and excretion (ADME), as well as toxicity. While keeping the pharmacophore unchanged, these new molecules also possess the ability, like the original oligonucleotides, to suppress autoimmunity, acute or chronic inflammation, repair damaged organs or tissues, and alleviate and/or prevent pain (such as nociceptive, neuropathic, and other forms) in mammals, including humans.

In certain embodiments, the method of producing LLONCs involves the conjugation of amino-spacer modified oligonucleotides, preferably at 3′ end position, with molecules such as natural or semisynthetic polysaccharides like hyaluronic acid or its derivatives and salts, polyalkylene oxide polymers (p. eg. PEG), or lipidic acids.

### a.—Hyaluronic Acid Conjugates:

In a preferred embodiment, the reaction steps include providing an aqueous mixture of HA (preferable 5000≥≥Mw≥≥1 million), or a salt thereof, adjusting the pH of the mixture to between 4.0 and 6.0 by the addition of an acid, and then reacting the aqueous HA solution with a carbodiimide, the reacting step taking place in the absence of a primary amine as nucleophile or a polyanionic polysaccharide (other than HA). Preferably, the HA solution has a concentration of between about 0.1% and about 5%; the acid includes, preferably, hydrochloric acid; the carbodiimide is either a soluble monocarbodiimide or biscarbodiimide; and the molar equivalent ratio of the carbodiimide to the HA is equal to or greater than 5%.

Preferred monocarbodiimides, but not limited to, are the following: I-ethyl-3-(3-dimethylaminopropyl) carbodiimide (“EDC4”), cyclohexyl-B—(N-methylmorpholino) ethylcarbodiimide p-toluene-sulfonate (“CMC”) and, 1-(3-dimethylaminopropyl)-3-ethylcarbodiimide methiodide (“ETC”).

Carbodiimides are monofunctional carbodiimides and include, but not limited to: N-methyl-N′-tert-butylcarbodiimde, N,N′-diisopropylcarbodiimide, N,N′-dicyclohexylcarbodiimde, N,N′-ditert-butylcarbodiimde, N-cyclohexyl-N′-tert-butylcarbodiimde, N,N′-dibutylcarbodiimide, N,N′-diisobutylcarbodiimide, N-allyl-N′-propylcarbodiimde, N,N′-diallycarbodiimide, N, allyl-N′-cyclohexylcarbodiimide, N-crotyl-N′-cyclohexylcarbodiimide, N-ally-N′—(B-hydroxyethyl) carbodiimide, N-methyl-N′-propylcarbodiimide, N-propyl-N′tert-butylcarbodiimide, N-isopropyl-N′-tert-butylcarbodiimide, N-(a-dimethylaminopropyl)-N′tert-butylcarbodiimide, N—(B-bromoallyl)-N′-propylcarbodiimide, N—(B-bromoallyl)-N′-isopropylcarbodiimide, N—(B-bromoallyl)-N′-tert-butylcarbodiimide, N-(a-dimethylaminopropyl)-N′—(B-bromoallyl) carbodiimide, 1-ethyl-3-(6-benzyloxylcarbonylaminohexyl) carbodiimide, 1-(3-dimethylaminopropyl)-3-(6-benzoylaminohexyl)-carbodiimide and the like.

In another embodiment the coupling agent class can include but not be limited to: uronium salts like HATU, HBTU/TBTU, TATU, TCTU, TDBTU, HBPyU, HBPipU, TOTU, HOTU and HCTU; phosphonium salts such as PyBOP, PyBrOP, BOP, AOP, PyAOP TPTDP and PyClop; formamidinium salts like TPTU, TNTU, HSTU, HSPyU, TOTT, HOTT, TFFH, TCFH, TPyICU, PyCIU, DMC, CIP, CIB.

The introduction of the coupling agent generally causes the pH to increase. However, the reaction is monitored by a pH meter and an acid (preferably HCl) is added to keep the pH of the reaction-mixture between about 4.75 and 5.50. The reaction is allowed to proceed at room temperature for about 4 to 24 h (preferably, for sixteen hours). A scheme of this reaction is shown in FIG. 6

The reaction is produced with a very high yield and the non-reactive oligonucleotide can be reprocessed increasing the yield even more. In an easy chromatographic step (FIG. 7.A), the conjugated molecule can be purified from the original reactives, using a size exclusion or ion exchange columns with the methodology available in the state of the art. The complete process is very quick and relatively inexpensive yielding a pure new entity (FIG. 7.B.).

The methodology is very strong and does not require any modifications if the conjugation is made with different hyaluronic acid sizes and ON of different lengths or even with their modified backbone (phosphorotioated, E.g.)

### b.—Polyethylene Glycol Conjugates:

The PEG (FIG. 8) may have average molecular weight of 100 to 100,000 Daltons, specifically 500 to 70,000 Daltons, more specifically 2,000 to 50,000 Daltons, but it not limited thereto. In a particularly preferred embodiment, the molecular weight is about 20,000 daltons. In another preferred embodiment, the molecular weight is about 40,000 daltons. It can be a single linear molecule or a branched one. In another embodiment, PEG can be functionalized so as to form a PEG bearing a terminal carboxylic group able to link the amino-functionalized ON in presence of the same carbodiimides of point a.—According to one embodiment, the PEG may be functionalized PEG with a terminal carboxylic acid that can involve, but are not limited to, terminal groups like malonic, glutaric, adipic, pimelic, fumaric acid. In a preferred embodiment the coupling reaction is performed at pH 6 ensured by a buffer (preferably 2-(N-morpholino) ethanesulfonic-MES-buffer 0.1M) at room temperature for 16 hours. FIG. 9 shows a schematic example of a reaction for producing a PEG-ODN LLONC.

In another embodiment the PEG carboxylic group can be converted into the correspondent acyl chloride or the correspondent anhydride that can directly react in an anhydrous environment like anhydrous dichloromethane at room temperature with the amino group to achieve the amide link (FIG. 10). In another embodiment, PEG can be functionalized so as to form a PEG bearing a terminal anhydride that can also react with the amino-functionalized ON (FIG. 11). In one embodiment, an aldehyde terminated PEG can be coupled to amino-functionalized ON, via reductive amination in presence of sodium cyanoborohydride at pH 4-5, forming the correspondent secondary amine (FIG. 12). In a further embodiment PEG can bear a terminal isocyanate group that can easily react with amino-functionalized ON in anhydrous environment (FIG. 13).

The reaction is produced with a very high yield and the non-reactive oligonucleotide can be reprocessed, increasing the yield even more. In an easy chromatographic step (FIG. 14.A), the PEG-ON conjugated molecule can be purified from the original reactives, using a C18 RP or ion exchange columns with the methodology available in the state of the art. The complete process is very quick and relatively inexpensive yielding a pure new entity (FIG. 14.B.). The methodology is very strong and does not require any modifications if the conjugation is made with different PEG sizes and ON of different lengths or even with their modified backbone (phosphorotioated, p. Eg.)

### c.—Fatty Acid Conjugates:

The fatty acid component can include a long chain, alkyl-substituted fatty acid. The fatty acid component preferably includes greater than 12 carbon atoms and more preferably between 12 to 24 carbon atoms. The alkyl chain can be a straight chain, a branched chain, or cyclic chain, all of which can be, mono- or polyunsaturated, (conjugated or non-conjugated), and combinations thereof. It can also be a hydrocarbon chain of either a J2 prostanoid or a mammalian or non-mammalian fatty acid. e.g., myristic acid, palmitic acid, stearic acid, oleic acid, linoleic acid, linolenic acid, eicosatrienoic acid, arachidonic acid, eicosapentenoic acid, and docosatetraenoic acid. Also pentadecanoic acid; heptadecanoic acid; nonadecanoic acid; heneicosanoic acid; 9-trans-tetradecanoic acid, 14:1 T; 10-trans-pentadecanoic acid, 15:1 T; 9-trans-hexadecenoic acid, 16:1 T; 10-heptadecenoic acid, 17:1; 10-trans-heptadecenoic acid, 17:1 T; 7-trans-nonadecenoic acid, 19:1T; 10,13-nonadecadienoic acid, 19:2; 11-trans-eicosenoic acid, 20:1T; and 12-heneicosenoic acid, 21:1.

In other preferred embodiments, the fatty acid includes naturally occurring fatty acids; more specifically the ω-3 long chain polyunsaturated fatty acids. Referenced examples of fatty acids for use in the present invention include, but are not restricted to: lauric acid (n-dodecanoic acid), myristic acid (n-tetradecanoic acid), palmitic acid (n-hexadecanoic acid), stearic acid (n-octadecanoic acid), arachidic acid (n-eicosanoic acid), behenic acid (n-docosanoic acid), lignoceric acid (n-tetracosanoic acid), palmitoleic acid (cis-Δ9-hexadecenoic acid), oleic acid (cis-Δ9-octadecenic acid), linoleic acid (cis, cis-Δ9,Δ12-octadecadienoic acid, cis, trans-Δ9,Δ11-octadecadienoic acid, and trans, cis-Δ10, Δ12-octadecadienoic acid), linolenic acid (cis-Δ9,Δ12, Δ15-octadecatrienoic acid, cis, trans, cis-Δ9,Δ11, Δ13-octadecatrienoic acid, cis, trans, trans-Δ9,Δ11, Δ13-octadecatrienoic acid, and trans, trans, cis-Δ9,Δ11,Δ13-octadecatrienoic acid), and arachidonoic (cis-Δ5, Δ8,Δ11,Δ14-eicosatetraenoic acid), docosahexanoic acid (DHA), and eicosapentenoic acid (EPA).

In preferred embodiments, the fatty acids can be selected from the list shown in FIG. 15. In a preferred embodiment the coupling reaction between the carboxylic group of the lipidic acid and the amino group on the ON is performed at pH 6, ensured by MES buffer 0.1M at room temperature for 16 hours in presence of one of the condensing agents previously described at point a (Hyaluronic acid conjugates). FIG. 16 shows a schematic example of a reaction for producing a Palmitic Acid-ODN LLONC.

In another embodiment, the lipidic carboxylic group of docohexanoic acid can be converted into the correspondent acyl chloride or the correspondent anhydride that can directly react in an anhydrous environment like anhydrous dichloromethane at room temperature with the amino group to achieve the amide link (FIG. 17).

The reaction is produced with a very high yield and the non-reactive oligonucleotide can be reprocessed, increasing the yield even more. In an easy chromatographic step (FIG. 18.A), the Lipid-ON conjugated molecule can be purified from the original reactives, using a C18 RP or ion exchange columns with the methodology available in the state of the art. The complete process is very quick and relatively inexpensive yielding a pure new entity (FIG. 18.B.). The methodology is very strong and does not require any modifications if the conjugation is made with different lipids and ON of different lengths or even with their modified backbone (phosphorotioated, E.g.)

### Regarding Oligonucleotides:

A phosphodiester as well as a partially or fully phosphorothioated molecule are also embodied herein as typical backbone modifications of the conjugated oligonucleotide.

In certain embodiments, the conjugated oligonucleotide can be selected from a chain size ON from 12 to 100 nucleotides long.

There are hundreds of CpG ONs with different sequences but similar activity; so, variations from the model molecule of SEQ ID N^{o}: 1, can be equivalently use for synthetized LLONCs. Then:

In certain embodiments the active zone of the conjugated CpG oligonucleotide can have a 100% sequence identity to SEQ ID N^{o}: 1.

In certain embodiments the active zone of the conjugated CpG oligonucleotide can have at least a 90% sequence identity to SEQ ID N^{o}: 1.

In certain embodiments the active zone of the conjugated CpG oligonucleotide can have at least an 80% sequence identity to SEQ ID N^{o}: 1.

In certain embodiments the active zone of the conjugated CpG oligonucleotide can have at least a 50% sequence identity to SEQ ID N^{o}: 1.

In addition, there are also more than 50 PyNTTTTGT oligonucleotides with different sequences but similar activity; so, variations from the model molecule of SEQ ID N^{o}: 2, can be equivalently use for synthetized LLONCs. Then:

In certain embodiments the active zone of the conjugated PyNTTTTGT oligonucleotide can have a 100% sequence identity to SEQ ID N^{o}: 2.

In certain embodiments the active zone of the conjugated PyNTTTTGT oligonucleotide can have at least a 90% sequence identity to SEQ ID N^{o}: 2.

In certain embodiments the active zone of the conjugated PyNTTTTGT oligonucleotide can have at least a 80% sequence identity to SEQ ID N^{o}: 2.

In certain embodiments the active zone of the conjugated PyNTTTTGT oligonucleotide can have at least a 50% sequence identity to SEQ ID N^{o}: 2.

In certain embodiments where the oligonucleotides are conjugated with Hyaluronic acid. Hyaluronic acid size can be selected from a molecular weight range between 4 KDa to 1 MDa, such that the resulting conjugate contains at least a portion, retains or increase the original biological activity concerning to the oligonucleotide which forms the conjugate.

In certain embodiments where the oligonucleotides are conjugated with polyethylene glycol; polyethylene glycol size can be selected in the way that the molecular weight of the conjugate, excluding the weight of the ON, is between about 300 to about 40,000 daltons, such that the resulting conjugate contains at least a portion, retains or increase the original biological activity concerning to the oligonucleotide which forms the conjugate.

In certain embodiments where the oligonucleotides are conjugated with lipidic molecules, lipidic molecules employed can be selected between phospholipids, non-naturally occurring branched and unbranched fatty acids, and naturally occurring branched and unbranched fatty acids ranging from as few as 4 carbon atoms to more than 30 carbon atoms, such that the resulting conjugate contains at least a portion, retains or increase the original biological activity concerning to the oligonucleotide which forms the conjugate.

### LLONC Uses:

In certain embodiments, a composition comprises a therapeutically effective amount of any combination of different proportions of any type of LLONC described in this invention, and even free ON and/or long-lasting organic molecules.

In certain embodiments, a composition comprises a therapeutically effective amount of LLONCs disclosed in this invention, combined with one or more anti-inflammatory agents, immunosuppressive agents, chemotherapeutic agents, analgesic agent, antibiotic agent, anticoagulant agent, antidepressant agent, antiepileptic agent, antipsychotic agent, sedative agent, antiviral agent, cardiovascular agent, other chemotherapeutic or biotherapeutic agent or combinations thereof.

In certain embodiments, a composition comprises a therapeutically effective amount of LLONCs disclosed in this invention is directly administered into the body by enteral/gastroenteral, parenteral or topic routes.

In certain embodiments, a composition comprises a therapeutically effective amount of LLONCs disclosed in this invention is applied through an ex-vivo treatment, activating one or more immune cells. In certain embodiments, the immune cells comprise: B cells, T cells, antigen presenting cells or combinations thereof. In certain embodiments, the cells are autologous cells, comprising: autologous, allogeneic, haplotype matched, haplotype mismatched, haplo-identical, xenogeneic, cell lines or combinations thereof. In certain embodiments, after the ex-vivo activation of immune cells, the treatment comprises the administration of the activated cells or the supernatant of the treated cells or isolated/purified fractions of the treated cells or the isolated/purified fractions of the supernatant of the treated cells or combinations thereof.

In certain embodiments, a composition comprises a therapeutically effective amount of LLONCs disclosed in this invention is applied through an ex-vivo treatment, activating one or more stem cells. In certain embodiments, the cells are autologous cells, comprising: autologous, allogeneic, haplotype matched, haplotype mismatched, haplo-identical, xenogeneic, cell lines or combinations thereof. In certain embodiments, after the ex-vivo activation of immune cells, the treatment comprises the administration of the activated cells or the supernatant of the treated cells or isolated/purified fractions of the treated cells or the isolated/purified fractions of the supernatant of the treated cells or combinations thereof.

In certain embodiments, the suppression of the autoimmune response in a subject comprises the use (in-vivo or ex-vivo) of LLONC where the long-lasting organic molecules conjugated with the oligonucleotide is Hyaluronic Acid. In certain embodiments, the long-lasting organic molecules conjugated with the oligonucleotide is Polyethylene Glycol. In certain embodiments, the long-lasting organic molecules conjugated with the oligonucleotide is a lipidic molecule, thereby suppressing the autoimmune response in the subject.

In certain embodiments, the suppression of acute or chronic inflammation or repairing a damaged organ or tissue in a subject comprises the use (in-vivo or ex-vivo) of LLONC where the long-lasting organic molecules conjugated with the oligonucleotide is Hyaluronic Acid. In certain embodiments, the long-lasting organic molecules conjugated with the oligonucleotide is Polyethylene Glycol. In certain embodiments, the long-lasting organic molecules conjugated with the oligonucleotide is a lipidic molecule, thereby suppressing the autoimmune response in the subject.

In certain embodiments, the cancer treatment comprises the use (in-vivo or ex-vivo) of LLONC where the long-lasting organic molecules conjugated with the oligonucleotide is Hyaluronic Acid. In certain embodiments, the long-lasting organic molecules conjugated with the oligonucleotide is polyethylene glycol. In certain embodiments, the long-lasting organic molecules conjugated with the oligonucleotide is a lipidic molecule, thereby suppressing the autoimmune response in the subject.

In certain embodiments, the cancer treatment adjuvancy comprises the use (in-vivo or ex-vivo) of LLONC where the long-lasting organic molecules conjugated with the oligonucleotide is Hyaluronic Acid. In certain embodiments, the long-lasting organic molecules conjugated with the oligonucleotide is Polyethylene Glycol. In certain embodiments, the long-lasting organic molecules conjugated with the oligonucleotide is a lipidic molecule, thereby suppressing the autoimmune response in the subject.

In certain embodiments, the treatment for immune response regulation (e. g. autoimmunity, immunodepression, immunodeficiency) comprises the use (in-vivo or ex-vivo) of LLONC where the long-lasting organic molecules conjugated with the oligonucleotide is hyaluronic acid. In certain embodiments, the long-lasting organic molecules conjugated with the oligonucleotide is polyethylene glycol. In certain embodiments, the long-lasting organic molecules conjugated with the oligonucleotide is a lipidic molecule, thereby suppressing the autoimmune response in the subject.

In certain embodiments, the graft versus host disease prevention comprises the use (in-vivo or ex-vivo) of LLONC where the long-lasting organic molecules conjugated with the oligonucleotide is hyaluronic acid. In certain embodiments, the long-lasting organic molecules conjugated with the oligonucleotide is Polyethylene Glycol. In certain embodiments, the long-lasting organic molecules conjugated with the oligonucleotide is a lipidic molecule, thereby suppressing the autoimmune response in the subject.

In certain embodiments, the cytokine storm syndrome caused by viral infections (Influenza virus, Dengue virus, Coronavirus, Hantavirus, just as an example.) treatment comprises the use (in-vivo or ex-vivo) of LLONC where the long-lasting organic molecules conjugated with the oligonucleotide is Hyaluronic Acid. In certain embodiments, the long-lasting organic molecules conjugated with the oligonucleotide is Polyethylene Glycol. In certain embodiments, the long-lasting organic molecules conjugated with the oligonucleotide is a lipidic molecule, thereby suppressing the autoimmune response in the subject.

In certain embodiments, the sepsis treatment comprises the use (in-vivo or ex-vivo) of LLONC where the long-lasting organic molecules conjugated with the oligonucleotide is Hyaluronic Acid. In certain embodiments, the long-lasting organic molecules conjugated with the oligonucleotide is Polyethylene Glycol. In certain embodiments, the long-lasting organic molecules conjugated with the oligonucleotide is a lipidic molecule, thereby suppressing the autoimmune response in the subject.

In certain embodiments, the neurodegenerative disease treatment comprises the use (in-vivo or ex-vivo) of LLONC where the long-lasting organic molecules conjugated with the oligonucleotide is hyaluronic acid. In certain embodiments, the long-lasting organic molecules conjugated with the oligonucleotide is polyethylene glycol. In certain embodiments, the long-lasting organic molecules conjugated with the oligonucleotide is a lipidic molecule, thereby suppressing the autoimmune response in the subject.

In certain embodiments, the inflammatory disease treatment comprises the use (in-vivo or ex-vivo) of LLONC where the long-lasting organic molecules conjugated with the oligonucleotide is Hyaluronic Acid. In certain embodiments, the long-lasting organic molecules conjugated with the oligonucleotide is polyethylene glycol. In certain embodiments, the long-lasting organic molecules conjugated with the oligonucleotide is a lipidic molecule, thereby suppressing the autoimmune response in the subject.

In certain embodiments, the acute or chronic pain (Nociceptive, Neuropathic, and “other”-Nocipathic or Nociplastic pain-) treatment or prevention comprises the use (in-vivo or ex-vivo) of LLONC where the long-lasting organic molecules conjugated with the oligonucleotide is hyaluronic acid. In certain embodiments, the long-lasting organic molecules conjugated with the oligonucleotide is polyethylene glycol. In certain embodiments, the long-lasting organic molecules conjugated with the oligonucleotide is a lipidic molecule, thereby suppressing the autoimmune response in the subject.

In certain embodiments, the aging condition treatment comprises the use (in-vivo or ex-vivo) of LLONC where the long-lasting organic molecules conjugated with the oligonucleotide is hyaluronic acid. In certain embodiments, the long-lasting organic molecules conjugated with the oligonucleotide is polyethylene glycol. In certain embodiments, the long-lasting organic molecules conjugated with the oligonucleotide is a lipidic molecule, thereby suppressing the autoimmune response in the subject.

In certain embodiments, the LLONC can be used to form a coating on an implantable device, which may be used alone or include a bioactive agent. Representative bioactive agents include, anticancer drugs, anti-inflammatory, homeostatic, anticoagulants, analgesics, only as examples.

The compositions provided herein, in certain embodiments, can be coated onto an implantable device. The implantable device can be any implantable device. In one embodiment, the implantable device is a drug-delivery stent. The implantable device can be used for the treatment of a medical condition such as athero-sclerosis, thrombosis, restenosis, high cholesterol, hemorrhage, vascular dissection or perforation, vascular aneurysm, vulnerable plaque, chronic total occlusion, claudication, anastomotic proliferation for vein and artificial grafts, bile duct obstruction, ureter obstruction, tumor obstruction, and combinations thereof. In one embodiment, the implantable device can be an implantable cardioverter defibrillator, artificial hip, heart pacemaker, artificial or replacement bones, breast implant, spine screw, artificial disc, intra-uterine device, metal screw, pin, plate, or rod (traumatic fracture repair), artificial knee, coronary stent, vessel stent, ear tube, artificial eye lenses, biological sensor, just as examples.

Other embodiments are described infra.

### Definitions

The terminology used herein is only for the purpose of describing particular embodiments and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments±50%), in some embodiments±20%, in some embodiments±1.0%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

The term “Aging”, as used herein, refers to those progressive physiological changes in an organism that lead to senescence, or a decline of biological functions and of the organism's ability to adapt to metabolic stress. Aging takes place in a cell, an organ, or the total organism with the passage of time. It is a process that goes on over the entire adult life span of any living thing.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

The term “autoimmunity,” as used herein, refers to the failure of an organism to recognize its own constituent parts as self, resulting in an immune response against the organism's own cells and tissues. “Autoimmune disease” refers to any diseases caused by autoimmunity. Examples of autoimmune diseases are: Achalasia, Addison's disease, adult still's disease, agammaglobulinemia, alopecia areata, amyloidosis, ankylosing spondylitis, anti-GBM/anti-TBM nephritis, Antiphospholipid syndrome, Autoimmune angioedema, Autoimmune dysautonomia, Autoimmune encephalomyelitis, Autoimmune hepatitis, autoimmune inner ear disease (AIED), Autoimmune myocarditis, autoimmune oophoritis, autoimmune orchitis, autoimmune pancreatitis, autoimmune retinopathy, autoimmune urticaria, Axonal & neuronal neuropathy (AMAN), Baló disease, Behcet's disease, benign mucosal pemphigoid, Bullous pemphigoid, Castleman disease (CD), celiac disease, Chagas disease, Chronic inflammatory demyelinating polyneuropathy (CIDP), chronic recurrent multifocal osteomyelitis (CRMO), Churg-Strauss Syndrome (CSS) or eosinophilic granulomatosis (EGPA), cicatricial pemphigoid, Cogan's syndrome, cold agglutinin disease, Congenital heart block, Coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), fiant cell myocarditis, flomerulonephritis, Goodpasture's syndrome, franulomatosis with polyangiitis, Graves' disease, Guillain-Barre syndrome, Hashimoto's thyroiditis, hemolytic anemia, Henoch-Schonlein purpura (HSP), Herpes gestationis or pemphigoid gestationis (PG), hidradenitis suppurativa (HS) (acne inversa), hypogammalglobulinemia, IgA Nephropathy, IgG4-related sclerosing disease, Immune thrombocytopenia purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, Lyme disease chronic, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, perivenous encephalomyelitis, Pernicious anemia (PA), POEMS syndrome, polyarteritis nodosa, polyglandular syndromes type I, II, III, polymyalgia rheumatica, polymyositis, postmyocardial infarction syndrome, Postpericardiotomy syndrome, Primary biliary cirrhosis, Primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm & testicular autoimmunity, stiff person syndrome (SPS), subacute bacterial endocarditis (SBE), Susac's syndrome, sympathetic ophthalmia (SO), Takayasu's arteritis, temporal arteritis/giant cell arteritis, thrombocytopenia purpura (TTP), Tolosa-Hunt syndrome (THS), transverse myelitis, Type 1 diabetes, ulcerative colitis (UC), undifferentiated connective tissue disease (UCTD), uveitis, vasculitis, vitiligo, Vogt-Koyanagi-Harada disease 72.

The term as used herein, “Pain” refers (last definition proposed by IASP's, 2019-https://www.iasp-pain.org/PublicationsNews/NewsDetail.aspx?ItemNumber=9218-) to “An unpleasant sensory and emotional experience associated with actual or potential tissue damage, or described in terms of such damage. It is also contained under “Neurological Disorders” definition, but with entity enough for being treated independently. There are two well-recognized broad categories of pain: the common sensical sort (the pain of damage), and the somewhat kind that comes from damage to the system that reports and interprets damage, the nervous system. More specifically:
1. Nociceptive pain arises from various kinds of trouble in tissues, reported to the brain by the nervous system^{xxxvii}. This is the type of pain everyone is most familiar with, everything from bee stings and burns and toe stubs to repetitive strain injury, nausea, tumours, and inflammatory arthritis. Nociceptive pain typically changes with movement, position, and load. Represents the normal response to noxious insult or injury of tissues such as skin, muscles, visceral organs, joints, tendons, or bones.
   Examples include:
a. Somatic: musculoskeletal (joint pain, myofascial pain), cutaneous; often well localized
b. Visceral: hollow organs and smooth muscle.
2. Neuropathic pain arises from damage to the nervous system itself, central or peripheral, either from disease, injury, or pinching; Formally: “Pain caused by a lesion or disease of the somatosensory nervous system.” Neuropathic pain is caused by a lesion or disease of the somatosensory system, including peripheral fibres (Aβ, Aδ and C fibres) and central neurons, and affects 7-10% of the general population (xxxviii). IASP has recently [2008] published a new definition of neuropathic pain according to which neuropathic pain is defined as ‘pain caused by a lesion or disease of the somatosensory system.’ The simplest neuropathies are mechanical insults: anything that damages neurons, from (e.g.) multiple sclerosis to chemotherapy to alcoholism to phantom limb pain. It's often stabbing, electrical, or burning, but nearly any quality of pain is possible. Unfortunately, it's also more likely to lead to chronic pain, because nerves don't heal appropriately (⁷⁵).

Lesions or diseases of the somatosensory nervous system can lead to altered and disordered transmission of sensory signals into the spinal cord and the brain; common conditions associated with neuropathic pain include postherpetic neuralgia, trigeminal neuralgia, painful radiculopathy, diabetic neuropathy, HIV infection, leprosy, amputation, peripheral nerve injury pain and stroke (in the form of central post-stroke pain). Triggers that lead to corneal nerve damage result in corneal neuropathy. Damage to nerves during refractive surgeries, ocular surface diseases such as chronic dry eye disease, recurrent corneal erosions, corneal neuropathic infections such as herpetic simplex and zoster, systemic neuropathic conditions such as diabetes, exposure to topical and systemic drugs, radiation keratopathy and chemotherapy (^{76, 77}).

Some common kinds of pain are not a great fit for either of the two official categories. The canonical example is the pain of fibromyalgia. [Mayo] Historically they have often been called “functional pain disorders” (⁷⁸). Other major examples:
a. complex regional pain syndrome (CRPS) [Mayo] and the closely related amplified pain syndome [Cleveland Clinic]
b. nonspecific chronic low-back pain
c. primary (unexplained) headache
d. irritable bowel syndrome [Mayo] and other chronic visceral pain disorders
e. conditions that begin as nociceptive pain, like osteoarthritis, but then go into a hellish downward spiral of sensitization.

Fibromyalgia is probably a pain system dysfunction, a poorly understood multi-system failure causing widespread body pain, but “dysfunction” of the nervous system is specifically excluded from neuropathic pain, by decree, as of. Dysfunction means that fibromyalgia isn't caused by any (known) damage to the nervous system.

It's plausible that both, small fibre peripheral neuropathy and positional cervical cord compression, are candidate neuropathic etiologies; both, hard to detect, able to explaining at least some of the symptoms of fibromyalgia, both associated with people who have diagnosed with fibromyalgia. Probably, this is a dysfunction, coming from widespread problems in a complex system (xxxix,xl).

Obviously, these kinds of pain can and do overlap. Some medical problems, like injuries, can affect both nerves themselves and other tissues, causing both kinds of pain; really, most pain does involve elements of these three types (xli).

As used herein, the term “cancer therapy” refers to a therapy useful in treating cancer. Examples of anti-cancer therapeutic agents include, but are not limited to, antibacterial agents as described herein as well as, e.g., surgery, chemotherapeutic agents, immunotherapy, growth inhibitor-agents, cytotoxic agents, agents used in radiation therapy, anti-angiogenesis agents, apoptotic agents, anti-tubulin agents, and other agents to treat cancer, such as:

A “biotherapeutic agent” is a chemical compound useful in the treatment of cancer. Examples of biotherapeutic agents are epidermal growth factor receptor (EGFR) antagonist (e.g., a tyrosine kinase inhibitor), anti-VEGF receptor(s) antibodies (e.g., bevacizumab, ranibizumab), anti-von Willebrand factor (VWF) antibodies (e.g., caplacizumab), anti-CD20 antibodies (e.g., ocrelizumab), anti-CD3 antibodies (e.g., blinatumomab), anti-CD3 antibodies (e.g., blinatumomab), anti-CD3/EpCAM antibodies (e.g., catumaxomab), anti-CD30 antibodies (e.g., brentuximab), anti-CD79-b antibodies (e.g., polatuzumab), anti-EGFR antibodies (e.g., cetuximab), anti-GD2 antibodies (e.g., dinutuxiximabeta), anti-GD22 antibodies (e.g., inutuzumabozogamicin), anti-HER-2 antibodies (e.g., ado trastuzumab), anti-IL-23 antibodies (e.g., guselkumab), anti-IL-4/IL-13 antibodies (e.g., dupilumab), anti-IL-5R antibodies (e.g., benralizumab), anti-IL-6R antibodies (e.g., sarilumab), anti-PD-L1 antibodies (e.g., avelumab, durvalumab), COX-2 inhibitor (e.g., celecoxib), HERI/EGFR inhibitor (e.g., erlotinib), platelet derived growth factor inhibitors (e.g., Imatinib Mesylate), interferons, cytokines, antagonists (e.g., neutralizing antibodies) that bind to one or more of the following targets ErbB2, ErbB3, ErbB4, PDGFR-beta, BlyS, APRIL, BCMA receptor(s), TRAIL/Apo2, and other bioactive and organic chemical agents, etc. Combinations thereof are also contemplated for use with the methods described herein.

A “chemotherapeutic agent” is a chemical compound useful in the treatment of cancer. Examples of chemotherapeutic agents include A “chemotherapeutic agent” is a chemical compound useful in the treatment of cancer. Examples of chemotherapeutic agents include 2,2′,2″-trichiorotriethylamme; 2-ethylhydrazide; 5-FU (5-fluorouracil); 6-azauridine; 6-diazo-5-oxo-L-norieucine; 6-mercaptopurine; 6-thioguanine; aceglatone; acetogenins (especially buliataem and bullatacinone); aclacinomysins; actinomycin; AG1478; AG1571; albumin-engineered nanoparticle formulation of paclitaxel; aldophosphannde glycoside; alkyl sulfonates such as busulfan, improsulfan and piposuifan; alkylating agents such as thiotepa and cyclosphosphamide; aminolevulinic acid; aminopterin; amsacrine; and doxetaxel; androgens such as calusterone; anthramycin; anti-adrenals such as aminoglutethimide; antibiotics such as the enediyne antibiotics (e.g. calicheamicin); anti-metabolites such as methotrexate and 5-fluorouracil (5-FU); arabinoside (“Ara-C”); azacytidine; azaserine; aziridmes such as benzodopa, carboquone, meturedopa and uredopa; bestrabucil; bisantrene; bisphosphonates such as clodronate; bleomycins; bortezomib; bryostatin; cactinomycin; callystatin; caminomycin; camptothecin (including the synthetic analogue topotecan); capecitabine; carabicin; carmofur; carzinophilin; CC-1065 (including its adozcicsin, carzcicsin and bizcicsin synthetic analogues); chloranbucil; chlornaphazine; cholophosphamide; chromomycinis; chromoprotein enediyne antibiotic chromophores; CPT-11; cremophor-free; cryptophycins (particularly cryptophycin 1 and ciyptophycin 8); cyclophosphamide; cytarabine; dacarbazine; dactinomycin; daunomycin; daunorubicin; defofamine; demecolcine; detorubicin; diaziquone; dideoxyuridme; difluoromethylornithine (DMFO); dolastatin; doxifluridine; doxorubicin; dromostanolone propionate; duocarmycin (including the synthetic analogues KW-2189 and CB1-TM1); dynemicin including dynemicin a; edatraxate; edatrexate; eieutherobin; elfomithine; elliptinium acetate; emluracii; enocitabine; epirubicin; epitiostanol; epothilone; erlotinib; esorubicin; esperamicin; estramustine; ethylenimines and methylamelamines including altretarriine, triethylenemelamine, triethylenephosphoramide, triethylenethiophosphoramide and trimethylomelamme; etoglucid; etoposide (VP-16); floxuridine; folic acid analogues such as denopterin; folic acid replenisher such as frolinic acid; fulvestrant; gacytosinc; gallium; gefitimb; gemcitabine; hydroxyurea; ibandronate; idarubicin; ifosfamide; ifosfamide; imatinib mesylate; lapatimb; lentinan; letrozole; leucovorin; lonafarnib; lonidainine; losoxantrone; marcellomycin; maytansinoids such as maytansine and ansanntocins; mechlorethamine; mechlorethamine oxide hydrochloride; melphalan; mepitiostane; mercaptopurine: methotrexate; methotrexate; mitobronitol; mitoguazone; mitolactol; mitomycins such as mitomycin c; mitotane; mitoxantrone; mitoxantrone; mopidanmol; mycophenolic acid; neocarzinostatin chromophore; niannomustine; nitraerine; nitrate; nitrogen mustards such as chlorambucil; nitrosureas such as carmustine, chlorozotocm, fotemustine, lomustine, nimustine and ranimnustine; nogalamycin; novantrone; novembichin; olivomycins; oxaliplatin; paclitaxel; pancratistatin; pentostatin; peplomycin; phenamet; phenesterine; pipobroman; pirarubicin; platinum; platinum analogs such as cisplatin and carboplatin: vinblastine; podophyllinic acid; potfiromycm; prednimustine; procarbazine; pteropterin; PTK787/ZK 222584; purine analogs such as fiudarabine; puromycin; pyrimidine analogs such as ancitabine; quelamycin; rapamycin; razoxane; retinoids such as retinoic acid; rhizoxin; rodorubicin; sarcodictyin; sizofuran; sorafenib; spirogermanium; spongistatm; streptonigrin; streptozocin; sutent; taxoids; teniposide; tenuazonic acid; testolactone; thiamiprine; thioguanine; thiotcpa; topoisomerase inhibitor RFS 2000; triaziquone; trichothecenes; triiostane; trimetrexate; trofosfamide; tubercidin; ubemmcx; uracil mustard; urethan; vincristine; vindesme; vinorelhine; xeloda; zinostatin; zorubicin and pharmaceutically acceptable salts, acids or derivatives of any of the above.

Also included in this definition of “chemotherapeutic agent” are: (i) anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene; (ii) aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in aberrant cell proliferation, such as, for example, PKC-alpha, Ralf and H-Ras; (viii) ribozymes such as a VEGF expression inhibitor (e.g., ribozyme) and aHER2 expression inhibitor: (ix) vaccines such as gene therapy vaccines, anti-angiogenic agents such as bevacizumab; and (xi) pharmaceutically acceptable salts, acids or derivatives of any of the above.

The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used herein, the term “cytokine” refers genencally to proteins released by one cell population that act on another cell as intercellular mediators or have an autocrine effect on the cells producing the proteins. Examples of such cytokines include lymphokines, monokines; interleukins (“ILs”) such as IL-1, IL-la, IL-2, IL-3, IL-4, IL-5, IL-6, IL-7, IL-8, IL-9, IL-10, IL-11, IL-12, IL-13, IL-15, IL-17A-F, IL-18 to IL-29 (such as 1L-23), IL-31, IL-35, rIL-2; a tumor-necrosis factor such as TNF-□ or TNT-φ, TGF-π-3; and other polypeptide factors including leukemia inhibitory factor (“LIF”), ciliary neurotrophic factor (“CNTF”), CNTF-like cytokine (“CLC”), cardiotrophin (“CT”), and kit ligand (“KL”).

As used herein, the terms “cytokine storm” or “cytokine storm syndrome” refers to the result of an immune system gone wild. The body's own killer immune cells are often defective, resulting in increased production of inflammatory proteins that can lead to organ failure and death. Cytokine storm syndrome, or CSS, is an overly exuberant immune response to a triggering event, frequently certain viral infections, including deadly strains of Influenza virus, Dengue virus, Coronavirus, Hantavirus, just as an example. No one knows why some people—and not others—develop this response; but there are likely host risk factors, including genetic mutations in genes that contribute to a familial form of this disease.

Cytokine storms are associated with a wide variety of infectious and noninfectious diseases and have even been the unfortunate consequence of attempts at therapeutic intervention. Previous reviews have centered on the advent of the concept (xlii) or its role in graft-versus-host disease (xliii), multiple sclerosis (xliv), pancreatitis (xlv), or multiple organ dysfunction syndrome (xlvi). Though the term was not explicitly stated, recent reviews have addressed potential cellular and molecular mechanisms contributing to the cytokine storm in viral disease (xlvii, xlviii), some of which specifically focused on influenza. Here, the cytokine storm is focused in the context of infection, with particular emphasis on respiratory viruses.

Acute lung injury (ALI) is a common consequence of a cytokine storm in the lung alveolar environment and systemic circulation and is most commonly associated with suspected or proven infections in the lungs or other organs (xlix). In humans, ALI is characterized by an acute mononuclear/neutrophilic inflammatory response followed by a chronic fibroproliferative phase marked by progressive collagen deposition in the lung. Pathogen-induced lung injury can progress into ALI or its more severe form, acute respiratory distress syndrome (ARDS), as seen with SARS-COV, MERS, Dengue multiple infection, Ebola and influenza virus infections. IL-1B is a key cytokine driving proinflammatory activity in bronchoalveolar lavage fluid of patients with lung injury (I). Intense inflammation in the lungs also can have other systemic effects on other organs (li).

The cytokine storm is best exemplified by severe lung infections, in which local inflammation spills over into the systemic circulation, producing systemic sepsis, as defined by persistent hypotension, hyper- or hypothermia, leukocytosis or leukopenia, and often thrombocytopenia. Viral, bacterial, and fungal pulmonary infections all cause the sepsis syndrome, and these etiological agents are difficult to differentiate on clinical grounds and patients eventually passed away because of multiple organ dysfunction syndrome (MODS), as a consequence of a severe cytokine storm, leaded by IL6, which is mainly used as a severity disease marker. In some cases, persistent tissue damage without severe microbial infection in the lungs also is associated with a cytokine storm and clinical manifestations that mimic sepsis syndrome. In addition to lung infections, the cytokine storm is a consequence of severe infections in the gastrointestinal tract, urinary tract, central nervous system, skin, joint spaces, and other sites (92).

Today, there are both broadly immunosuppressive approaches, such as high-dose corticosteroids and more novel targeted approaches that go after inflammatory cytokine proteins. These include the interleukins IL-1 and IL-6, and interferon-gamma. Systemic production of IL-10 following the onset of a cytokine storm is a marker of a counter-anti-inflammatory response that has been termed “immunoparalysis,” in that it is associated with downregulation of neutrophil and monocyte function in the systemic circulation. Downregulation of systemic inflammation might be conceptually beneficial in controlling systemic responses to local infections. However, it has been suggested that patients who survive the initial cytokine storm but subsequently die may be those who do not recover from immunoparalysis. Patients with persistent downregulation of HLA-DR (a marker of immunosuppression) on monocytes 3 to 4 days after the onset of severe sepsis and cytokine storm have a high mortality rate, suggesting a rationale for therapy to reverse immunosuppression under such circumstances (62).

Across much of the world, infectious diseases remain a very real threat, accounting for approximately half of all deaths. Malaria, tuberculosis, HIV disease, influenza, dengue, and endemic and emerging infections all contribute to morbidity and mortality. In addition to the emergence of new diseases, the continued rise of drug resistance among all the major infections is outpacing the rate of discovery of new antibiotics. Against this backdrop of antimicrobial resistance and the emergence of new pathogens, increasing interest has focused on the development of drugs that target the immune response to infection. Many acute infections are characterized by a powerful and potentially destructive immune response, and it would seem logical to target this response in order to reduce the self-inflicted damage initiated by the host in response to infection (lii).

Interestingly, in the lungs, the location of the initial infection does not seem to be a determinant of the severity of local and systemic cytokine storms. For example, influenza viruses infect and destroy the ciliated epithelial cells of the conducting airways, whereas SARS-COV infects type II pneumocytes in the alveolar walls, and hantavirus particles infect microvascular endothelial cells in the alveolar walls, yet all can lead to indistinguishable clinical syndromes of acute lung injury (ALI) with respiratory failure, sepsis, and a cytokine storm (62). Dengue virus, is able to infect human skin dendritic cells producing an increased vascular permeability, hemorrhagic manifestations, thrombocytopenia, hepatomegaly and a final circulatory failure which are due, in its severe form, to the occurrence of a cytokine storm (liii).

Yet to date, successful targeting of the immune system during acute infections has proved to be extraordinarily difficult and largely unsuccessful. However, new ONs, mainly PyNTTTGT ONs and new LLONC derivatives appears to be a good potential drug against this biphasic immune dysfunction, reaching easily the organism to hemostasis.

A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, a dosing regimen is or has been correlated with a desired therapeutic outcome, when administered across a population of patients.

As used herein, the term “immune cells” generally includes white blood cells (leukocytes) which are derived from hematopoietic stem cells (HSC) produced in the bone marrow “Immune cells” includes, e.g., lymphocytes (T cells, B cells, natural killer (NK) ceils) and myeloid-derived ceils (neutrophil, eosinophil, basophil, monocyte, macrophage, dendritic cells).

The term “Immunomodulatory oligonucleotides” refers to short sequences of synthetic DNA or RNA molecules that possess the ability to modulate the immune system. They are engineered to mimic certain patterns or motifs which once introduced into the body, they activate an adequate immune response. These oligonucleotides find applications across various domains of immunology and medical research, including vaccine development, immunotherapy, and also the investigation of immune responses.

Upon introduction into the body, immunomodulatory oligonucleotides set off a series of signaling events that culminate in immune system regulation. This regulation encompasses the production of proinflammatory cytokines and the recruitment of immune cells to the injection site. Eventually, as modulator of the immune response, they can also act as promotors of anti-inflammatory reaction, being an excellent tool against allergy.

Immunostimulatory oligonucleotides can enhance the presentation of antigens to immune cells, such as dendritic cells. This enhancement contributes to an improved immune response to vaccines by increasing the efficiency of antigen uptake and processing.

immunostimulatory oligonucleotides are synthetic NA sequences designed to activate the immune system. Their ability to trigger immune responses makes them important tools in immunology research and valuable components in vaccine development and immunotherapy.

Two different families are well distinguished:

“immunomodulatory CpG containing oligonucleotide”, or “CpG ODN” refer to an oligonucleotide, which contains a cytosine, guanine dinucleotide sequence and stimulates (e.g. has a mitogenic effect) on vertebrate lymphocyte. Preferred immunostimulatory oligonucleotides are between 2 to 100 base pairs in size and contain a consensus mitogenic CpG motif represented by the formula:

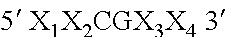

wherein C and G are unmethylated, X₁, X₂, X₃ and X₄ are nucleotides and a GCG trinucleotide sequence is not present at or near 5′ and 3′ termini.

Preferably the immunomodulatory oligonucleotides range between 8 to 40 base pairs in size. In one preferred embodiment, X₁X₂ is the dinucleotide GpA. In another preferred embodiment, X₃X₄ is preferably the dinucleotide TpC or also TpT. In a particularly preferred embodiment, the consensus motif X₁X₂CGX₃X₄ is preceded on the 5′ end by a T. Particularly preferred consensus sequences are TGACGTT or TGACGTC.

“Non CpG immunostimulatory oligonucleotides: They are oligonucleotides containing the non-palindromic sequence motif:

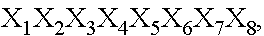

wherein X₁ is C, T, G or A (preferably T or C); X₂ is C, T, G or A; X₇ is C, T, G or A (preferably G); at least three, and preferably all, of X₃, X₄, X₅, X₆ and X₈ are T; with the proviso that, in the motif, a C does not precede a G (in other terms, the nucleic acid motif does not consist of a CpG oligonucleotide), which modulate the immune response. This immune modulation is characterized by stimulation of proliferation, differentiation, cytokine production and antibody production on B-cells and cell differentiation, cytokine production and antibody production on B-cells and cell differentiation on plasmacytoid dendritic cells. These activities allow the use of these molecules for controlling inflammatory as well as anti-inflammatory processes in the body. They also induce in vivo expansion of MSCs, resulting in a marked improvement in preclinical models of neuropathic pain, osteoporosis, diabetes and sepsis. Immunoprotective immunoregenerative therapy.

As used herein, the term “Lipids” refers to cholesterol (and thio analogs thereof), cholic acid, cholanic acid, lithocholic acid, adamantane acetic acid, 1-pyrene butyric acid, dihydrotestosterone, glycerol (e.g., esters (e.g., mono, bis, or tris fatty acid esters, e.g., C10, C11, C12, C13, C14, C15, C16, C17, C18, C19, or C20 fatty acids) and ethers thereof, e.g., C10, C11, C12, C13, C14, C15, C16, C17, C18, C19, or C20 alkyl; e.g., 1,3-bis-O (hexadecyl)glycerol, 1,3-bis-O (octaadecyl)glycerol), geranyloxyhexyl group, hexadecylglycerol, borneol, menthol, 1,3-propanediol, heptadecyl group, palmitic acid, stearic acid (e.g., glyceryl distearate), oleic acid, myristic acid, O3-(oleoyl) lithocholic acid, O3-(oleoyl) cholenic acid, dimethoxytrityl, or phenoxazine), a thioether, e.g., hexyl-S-tritylthiol, a thiocholesterol, an aliphatic chain, e.g., dodecandiol or undecyl residues, a phospholipid, e.g., di-hexadecyl-rac-giycerol or triethylammonium 1,2-di-0-hexadecyi-rac-glycero-3-H-phosphonate.

As used herein, the term, “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject which had, has, or is susceptible to cancer. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

The term “inflammation”, as used herein, refers to a local response to cellular injury that is characterize by capillary dilatation, leukocyte infiltration, redness, heat, and pain and that serves as a mechanism initiating the elimination of noxious agents and of damaged tissue. Inflammation is normally a self-limited process avoiding unnecessary′ organic damage. “Inflammatory disease” is a medical condition characterized by exaggerated or chronic inflammation. Autoimmune diseases are generally also inflammatory diseases. However, numerous inflammatory diseases are not consider autoimmune diseases. Examples of inflammatory diseases are Sepsis, Alzheimer, ankylosing spondylitis, arthritis, asthma, atherosclerosis, colitis, dermatitis, diverticulitis, fibromyalgia, hepatitis, irritable bowel syndrome, nephritis, Parkinson, Sclerosis multiple, bipolar disorder, autism, type-2 diabetes, osteoporosis and obesity.

By the term “modulate,” it is meant that any of the mentioned activities, are, e.g., increased, enhanced, increased, agonized (acts as an agonist), promoted, decreased, reduced, suppressed blocked, or antagonized (acts as an agonist). Modulation can increase activity more than 1-fold, 2-fold, 3-fold, 5-fold, 10-fold, 100-fold, etc., over baseline values. Modulation can also decrease its activity′ below baseline values. Modulation can also normalize an activity to a baseline value.

The term “modulator” is used to refer to an entity or agent whose presence in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate agent that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level.

As used herein, the term “neurodegenerative diseases” refers to a therapy useful in preventing and/or treating acute or chronic neurodegenerative diseases. Examples of neurodegenerative diseases are Alzheimer's disease (AD), Amyotrophic lateral sclerosis (ALS, Lou Gehrig's disease), Apoplexy, Cancers, Charcot-Marie-Tooth disease (CMT), Chronic traumatic encephalopathy, Cystic fibrosis, some cytochrome c oxidase deficiencies (often the cause of degenerative Leigh syndrome), Dementia, Ehlers-Danlos syndrome, Fibrodysplasia ossificans progressive, Friedreich's ataxia, Frontotemporal dementia (FTD), Guilin-Barre syndrome, some cardiovascular diseases (e.g. atheroscleroticones like coronary artery disease, aortic stenosisetc.), Huntington's disease, Infantile neuroaxonal dystrophy, Keratoconus (KC), Keratoglobus, Leukodystrophies, Lewy body disease, Lou Gehrig's disease, Macular degeneration (AMD), Marfan's syndrome (MFS), some mitochondrial myopathies, Mitochondrial DNA depletion syndrome, Multiple sclerosis (MS), Multiple system atrophy, Muscular dystrophies (MD), Neuronal ceroid lipofuscinosis, Niemann-Pick diseases, Osteoarthritis, Osteoporosis, Parkinson's disease, Pulmonary arterial hypertension, all prion diseases (Creutzfeldt-Jakob disease, fatal familial insomnia etc.), progressive dementia caused by gradual neuronal death and progressive Ataxia, Progressive supranuclear palsy, Retinitis pigmentosa (RP), Rheumatoid arthritis, Sandhoff Disease, Spinal muscular atrophy (SMA, motor neuron disease), Subacute sclerosing panencephalitis, Tay-Sachs disease, other forms of degenerative dementia, like final symptoms of Vascular dementia (e.g. brain stroke or cerebral ischemia).

As used herein, the term “Neurodegenerative Diseases Therapy” refers to a therapy useful in preventing and/or treating Neurodegenerative Diseases. Examples of Neurodegenerative Diseases therapeutic agents include, but are not limited to (R)-BPAP; 4-Cl-kynurenine; A-134974; A-35380; A-366833; AC-184897; AC-90222; ADCI; AEG-3482 series; AGY-110; AGY-207; AK-275; Alaptid; ALE-0540; Aloracetam; AlphaRx; AM-36; AMPA antagonists (Annovis); AMPAKINES; Amyloid-inhibiting peptides; Anapsos; Andrographolide; APBPI-124; Apoptosin; Aptiganel; AR-139525; AR-A-008055; Aricept (Donepezil); AR-R-17779; AR-R18565; ARRY-142886; ARX-(2000, -2001, -2002); AS-004509; AS-600292; AS-601245; a-Tocopherol; AutoVac; Axokine; AZ-36041; BA-1016; BAY-X-9227; BD-1054; BLS-602; BLS-605; BLSI; Breflate; BTG-A derivatives; C60 Fullerenes; CAS-493; Celecoxib; CEP-1347; CEP-3122; CEP-4143; CHF-2060; Clioquinol; Clk-1; CNIC-568; CNS-2103; CNS-5065; Copaxone; CP-132484; CP-283097; CPC-304; CX-516; Cyclo-phosphamide; Cyclosporin A; DCG-IV; DD-20207; Dehydroascorbic acid; Dexefaroxan; Dihydro-quinolines; Diperdipine; Dizocilpine; DP-103; DP-109; DP-b99; Dykellic acid; E-2101; EAβ-318; EF-7412; EGIS-7444; EHT-202; EP-475; Epigallocatechin-3-gallate; EQA-00; EQA-00, Anapsos; Ersofermin; ES-242-1; Estrogen; Estrogen/Progesterone; Ethanoanthracene derivatives; F-10981; F-2-CCG-I; FCE-29484A; FCE-29642A; FGF-9, rhuFGF-16; Fibroblast growth factor; Formobactin; FPL-16283; GAG mimetics; Galantamine derivatives; Galdansetron; Ganstigmine; GDNF; GGF-2; GKE-841; Glialines; GM-1 ganglioside; GM-1 ganglioside; GP-14683; GR-73632; GSK-3 inhibitors; GT-715; GV-2400; HBNF; HF-0220; HP-184; IAP; IDN-6556; IGF modulators; Imidazole derivatives; Imidazolyl nitrones; Inosine; Interferon Alpha; Interferon b; Interleukin-2-like growth factor; lometopane; Ipenoxazone; Isp-1; Istradefylline; Itameline; KF-17329; KP-102; KRX-411; KW-6002; L-687306; L-687414; L-689560; L-701252; Lamotrigine; LAU-0501; Leteprinim; Liatermine; LIGA-20; LY-178002; LY-233536; LY-235959; LY-302427; LY-354006; LY-354740; LY-451395; MCC-257; MDL-100748; MDL-102288; MDL-105519; MDL-27266; MDL-29951; MEM-1003; Memantine; Metallotexa-phyrins; Methylphenyle thynylpyridine (MPEP); Microalgal compound; Mirapex (pramipexole); MS-153; MT-5; N-3393; Naltrindole derivatives; NAPVSIPQ; NBI-30702; NC-531; Neotrofin; Neramexane; Nerve growth factor gene therapy; Neublastin; Neurocrine; Neurostrol; NNC-07-0775; Noggin; Norleu; NOX-700; NPS-1407; NRT-115; NS-1608; NS-2330; NXD-5150; NXY-059; Olanzapine; ONO-2506; OPC-14117; P-58; P-9939; PACAP; Palmidrol; Pan-Neurotrophin-1; PAN-811; PBT-1; PD-132026; PD-150606; PD-159265; PD-90780; PDC-008.004; PE21; Phenserine; Philanthotoxins; Piperidine derivatives; PK-11195 analogs; PN-277; PNU-101033E; PNU-157678; POL-255; PPI-368; PRE-103; PRS-211220; PYM-50028; QG-2283; Rasagiline; ReN-1820; Retigabine; RI-820; Rilutek (Riluzole)+ Dopamine agonist; RJR-1401; Ro-09-2210; RPR-104632; RS-100642; S-14820; S-176251; S-18986; S-33113-1; S-34730; S-34730-1; Safinamide; SB-277011; Selegiline; SEMAX; SIB-1553A; SIB-1553A; SIB-1765F; Siclofen; SJA-6017; SKF-74652; SL-34.0026; SNX-482; SP-(V5.2) C; SPC-9766; SPH-1371; SPM-914; SPM-935; SSR-180575; SSR-482073; Sumanirole; SUN-C5174; Survivins; SYM-2207; T-588; Talampanel; Talampanel; Taltirelin; TC-2559; TCH-346; Throphix; Thurinex; TK-14; Traxoprodil; Traxoprodil; U-74500A; UK-351666; UK-356297; UK-356464; Vanoxerine; Vasolex; VX-799; WAY-855; WIB-63480-2; WIN-67500; WIN-68100; WIN-69211; Xaliprodene; Ziconotide; Zonampanel and Zydis.

The term “Neurological Disorders” as used herein, refers to diseases of the central and peripheral nervous system. “Neurological condition or disease” refers to any diseases caused in the brain, spinal cord, cranial nerves, peripheral nerves, nerve roots, autonomic nervous system, neuromuscular junction, and muscles. Examples of neurological disorders are: Abulia, Achromatopsia, Acoustic neuroma, Agraphia, AIDS-neurological manifestations, Akinetopsia, Alcoholism, Alien hand syndrome, Allan-Herndon-Dudley syndrome, Alternating hemiplegia of childhood, Alzheimer's disease, Amaurosis fugax, Amnesia, Amyotrophic lateral sclerosis, Aneurysm, Angelman syndrome, Anosognosia, Aphasia, Aphantasia, Apraxia, Arachnoiditis, Arnold-Chiari malformation, Asomatognosia, Asperger syndrome, Ataxia, ATR-16 syndrome, Attention deficit hyperactivity disorder, Auditory processing disorder, Autism spectrum disorder, Behçet's disease, Bell's palsy, Bipolar disorder, Blindsight, Brachial plexus injury, Brain injury, Brain tumor, Brody myopathy, Canavan disease, Capgras delusion, Carpal tunnel syndrome, Causalgia, Central pain syndrome, Central pontine myelinolysis, Centronuclear myopathy, Cephalic disorder, Cerebral aneurysm, Cerebral arteriosclerosis, Cerebral atrophy, Cerebral autosomal dominant arteriopathy with subcortical infarcts and leukoencephalopathy, Cerebral dysgenesis-neuropathy-ichthyosis-keratoderma syndrome, Cerebral gigantism, Cerebral palsy, Cerebral vasculitis, Cerebrospinal fluid leak, Cervical spinal stenosis, Charcot-Marie-Tooth disease, Chiari malformation, Chorea, Chronic fatigue syndrome, Chronic inflammatory demyelinating polyneuropathy, Chronic pain, Cluster Headache, Cockayne syndrome, Coffin-Lowry syndrome, Coma, Complex regional pain syndrome, Compression neuropathy, Congenital distal spinal muscular atrophy, Congenital facial diplegia, Corticobasal degeneration, Cranial arteritis, Craniosynostosis, Creutzfeldt-Jakob disease, Cumulative trauma disorders, Cushing's syndrome, Cyclic vomiting syndrome, Cyclothymic disorder, Cytomegalic inclusion body disease, Cytomegalovirus Infection, Dandy-Walker syndrome, Dawson disease, De Morsier's syndrome, Dejerine-Klumpke palsy, Dejerine-Sottas disease, Delayed sleep phase disorder or syndrome, Dementia, Dermatomyositis, Developmental coordination disorder, Diabetic neuropathy, Diffuse sclerosis, Diplopia, Disorders of consciousness, Distal hereditary motor neuropathy type V, Distal spinal muscular atrophy type 1, Distal spinal muscular atrophy type 2, Down syndrome, Dravet syndrome, Duchenne muscular dystrophy, Dysarthria, Dysautonomia, Dyscalculia, Dysgraphia, Dyskinesia, Dyslexia, Dystonia, Empty sella syndrome, Encephalitis, Encephalocele, Encephalopathy, Encephalotrigeminal angiomatosis, Encopresis, Enuresis, Epilepsy, Epilepsy-intellectual disability in females, Erb's palsy, Erythromelalgia, Essential tremor, Exploding head syndrome, Fabry's disease, Fahr's syndrome, Fainting, Familial spastic paralysis, Fetal alcohol syndrome, Febrile seizures, Fisher syndrome, Fibromyalgia, Foville's syndrome, Fragile X syndrome, Fragile X-associated tremor/ataxia syndrome, Friedreich's ataxia, Frontotemporal dementia, Functional Neurological Disorder, Gaucher's disease, Generalized anxiety disorder, Generalized epilepsy with febrile seizures plus, Gerstmann's syndrome, Giant cell arteritis, Giant cell inclusion disease, Globoid cell leukodystrophy, Gray matter heterotopia, Guillain-Barre syndrome, Head injury, Headache, Hemicrania Continua, Hemifacial spasm, Hemispatial neglect, Hereditary motor neuropathies, Hereditary motor neuropathies, Hereditary spastic paraplegia, Heredopathia atactica polyneuritiformis, Herpes zoster, Herpes zoster oticus, Hirayama syndrome, Hirschsprung's disease, Holmes-Adie syndrome, Holoprosencephaly, HTLV-1 associated myelopathy, Huntington's disease, Hydranencephaly, Hydrocephalus, Hypercortisolism, Hypoalgesia, Hypoesthesia, Hypoxia, Immune-mediated encephalomyelitis, Inclusion body myositis, Incontinentia pigmenti, Infantile spasms, Inflammatory myopathy, Intracranial cyst, Intracranial hypertension, Isodicentric 15, Ischemic optic neuropathy, Joubert syndrome, Karak syndrome, Kearns-Sayre syndrome, Kinsbourne syndrome, Kleine-Levin syndrome, Klippel Feil syndrome, Krabbe disease, Kufor-Rakeb syndrome, Kugelberg-Welander disease-see Spinal muscular atrophy, Lafora disease, Lambert-Eaton myasthenic syndrome, Landau-Kleffner syndrome, Lateral medullary (Wallenberg) syndrome, Learning disabilities, Leigh's disease, Lennox-Gastaut syndrome, Lesch-Nyhan syndrome, Leukodystrophy, Leukoencephalopathy with vanishing white matter, Lewy body dementia, Lissencephaly, Locked-in syndrome, Lou Gehrig's disease-see Amyotrophic lateral sclerosis, Lumbar disc disease, Lumbar spinal stenosis, Lupus erythematosus-neurological sequelae, Lyme disease, Machado-Joseph disease, Macrencephaly, Macropsia, Macular degeneration, “Mal de debarquement”, Megalencephalic leukoencephalopathy with subcortical cysts, Megalencephaly, Melkersson-Rosenthal syndrome, Menieres disease, Meningitis, Menkes disease, Metachromatic leukodystrophy, Microcephaly, Micropsia, Migraine, Miller Fisher syndrome, Mini-stroke (transient ischemic attack), Misophonia, Mitochondrial myopathy, Mobius syndrome, Monomelic amyotrophy, Morvan syndrome, Motor neurone disease-Amyotrophic lateral sclerosis, Motor skills disorder, Moyamoya disease, Mucopolysaccharidoses, Multifocal motor neuropathy, Multi-infarct dementia, Multiple sclerosis, Multiple system atrophy, Muscular dystrophy, Myalgic encephalomyelitis, Myasthenia gravis, Myelinoclastic diffuse sclerosis, Myoclonic Encephalopathy of infants, Myoclonus, Myopathy, Myotonia congenita, Myotubular myopathy, Narcolepsy, Neuro-Behçet's disease, Neurofibromatosis, Neuroleptic malignant syndrome, Neuromyotonia, Neuronal ceroid lipofuscinosis, Neuronal migration disorders, Neuropathy, Neurosis, Niemann-Pick disease, Non-24-hour sleep-wake disorder, Nonverbal learning disorder, Occipital Neuralgia, Occult spinal dysraphism sequence, Ohtahara syndrome, Olivopontocerebellar atrophy, Opsoclonus myoclonus syndrome, Optic neuritis, Optic nerve disease, Optical manifestation related to Parkinson's and Alzheimer's diseases and cerebral stroke, Orthostatic hypotension, O'Sullivan-McLeod syndrome, Otosclerosis, Overuse syndrome, Palinopsia, PANDAS, Pantothenate kinase-associated neurodegeneration, Paramyotonia congenita, Paresthesia, Parkinson's disease, Paraneoplastic diseases, Paroxysmal attacks, Parry-Romberg syndrome, Pelizaeus-Merzbacher disease, Periodic paralyses, Peripheral neuropathy, Pervasive developmental disorders, Phantom limb/Phantom pain, Photic sneeze reflex, Phytanic acid storage disease, Pick's disease, Pinched nerve, Pituitary tumors, Polyneuropathy, PMG, Polio, Polymicrogyria, Polymyositis, Porencephaly, Post-polio syndrome, Postherpetic neuralgia, Posttraumatic stress disorder, Postural hypotension, Postural orthostatic tachycardia syndrome, Prader-Willi syndrome, Primary glaucomatous, Primary lateral sclerosis, Prion diseases, Progressive hemifacial atrophy, Progressive multifocal leukoencephalopathy, Progressive supranuclear palsy, Prosopagnosia, Pseudotumor cerebri, Quadrantanopia, Quadriplegia, Rabies, Radiculopathy, Ramsay Hunt syndrome type I, Ramsay Hunt syndrome type II, Ramsay Hunt syndrome type III-see Ramsay-Hunt syndrome, Rasmussen encephalitis, Reflex neurovascular dystrophy, Refsum disease, REM sleep behavior disorder, Repetitive stress injury, Restless legs syndrome, Retrovirus-associated myelopathy, Rett syndrome, Reye's syndrome, Rhythmic movement disorder, Romberg syndrome, Saint Vitus dance, Sandhoff disease, Sanfilippo syndrome, Schilder's disease (two distinct conditions), Schizencephaly, Sensory processing disorder, Septo-optic dysplasia, Shaken baby syndrome, Shingles, Shy-Drager syndrome, Sjögren's syndrome, Sleep apnea, Sleeping sickness, Snatiation, Sotos syndrome, Spasticity, Spina bifida, Spinal and bulbar muscular atrophy, Spinal cord injury, Spinal cord tumors, Spinal muscular atrophy, Spinal muscular atrophy with respiratory distress type 1-see Distal spinal muscular atrophy type 1, Spinocerebellar ataxia, Split-brain, Steele-Richardson-Olszewski syndrome-see Progressive supranuclear palsy, Stiff-person syndrome, Stroke, Sturge-Weber syndrome, Stuttering, Subacute sclerosing panencephalitis, Subcortical arteriosclerotic encephalopathy, Superficial siderosis, Sydenham's chorea, Syncope, Synesthesia, Syringomyelia, Tardive dyskinesia, Tarlov cyst, Tarsal tunnel syndrome, Tay-Sachs disease, Temporal arteritis, Temporal lobe epilepsy, Tetanus, Tethered spinal cord syndrome, Thalamocortical dysrhythmia, Thomsen disease, Thoracic outlet syndrome, Tic Douloureux, Tinnitus, Todd's paralysis, Tourette syndrome, Toxic encephalopathy, Transient ischemic attack, Transmissible spongiform encephalopathies, Transverse myelitis, Traumatic brain injury, Tremor, Trichotillomania, Trigeminal neuralgia, Tropical spastic paraparesis, Trypanosomiasis, Tuberous sclerosis, Unverricht-Lundborg disease, Vestibular schwannoma, Viliuisk encephalomyelitis, Visual Snow, Von Hippel-Lindau disease, Wallenberg's syndrome, Werdnig-Hoffmann disease-see Spinal muscular atrophy, Wernicke's encephalopathy, West syndrome, Whiplash, Williams syndrome, Wilson's disease, Y-Linked hearing impairment, Zellweger syndrome.

As used herein, the term “Neurological Diseases therapy”′ refers to a therapy useful in preventing and/or treating Neurological Diseases. Examples of Neurological Diseases therapeutic agents include, but are not limited to Calcium and colecalciferol, Alendronic acid (alendronate), Amantadine, Amitriptyline, Apomorphine, Azathioprine, Baclofen, Diazepam/Midazolam, Calcium and vitamin D, Carbamazepine, Clobazam, Clonazepam, Co-beneldopa, Co-careldopa, Colecalciferol (vitamin D), Cyclophosphamide, Diazepam, Dopamine agonists, Entacapone, Epistatus, Eslicarbazepine, Ethosuximide, Gabapentin, Lacosamide, Lamotrigine, Lansoprazole, Levetiracetam, Levodopa, Pyridostigmine, Methotrexate, Midazolam, Omeprazole, Oxcarbazepine, Phenobarbital, Phenytoin, Pramipexole, Prednisolone, Pregabalin, Primidone, Propantheline, Propranolol, Pyridostigmine, Ranitidine, Rasagiline, Retigabine, Riluzole, Ropinirole, Rotigotine, Selegiline, Sodium valproate, Entacapone/Tolcapone, Steroids, Rasagiline/Selegiline, Tolcapone, Topiramate, Verapamil, Vigabatrin, Vitamin D and Zonisamide.

As used in this specification and the appended claims, the term “or” is generally employed in its sense including “and/or” unless the content clearly dictates otherwise.

As used herein, the term “pain therapy” refers to a therapy useful in preventing and/or treating acute or chronic pain ((Nociceptive, Neuropathic, and “other”—Nocipathic or Nociplastic pain—). Examples of anti-pain therapeutic agents include, but are not limited to Corticosteroids, Opioids, Antidepressants, Anticonvulsants (anti-seizure medications), Nonsteroidal anti-inflammatory drugs (NSAIDs) and Lidocaine. Examples of Corticosteroids are prednisone, prednisolone, and methylprednisolone. Examples of Opioids are Codeine, Fentanyl, Hydrocodone, Hydrocodone-acetaminophen, Hydromorphone, Meperidine, Methadone, Morphine, Oxycodone, and Oxycodone-acetaminophen and Oxycodone plus naloxone. Some antidepressant medications include, e.g.: 1) Selective serotonin reuptake inhibitors (SSRIs) such as citalopram, fluoxetine, paroxetine, and sertraline. 2) Tricyclic antidepressants such as amitriptyline, desipramine, doxepin, imipramine, and nortriptyline. 3) Serotonin and norepinephrine reuptake inhibitors (SNRIs) such as venlafaxine and duloxetine. Some examples of Anticonvulsants (anti-seizure medications) include carbamazepine, gabapentin, and pregabalin. Examples of Nonsteroidal anti-inflammatory drugs (NSAIDs) are Diclofenac, Etodolac, Ibuprofen, Ketoprofen, Nabumetone, Naproxen, Oxaprosin and Paracetamol. Another means of topical pain relief comes in the form of a lidocaine.

The phrase “pharmaceutical acceptable carrier” refers to a carrier for the administration of a therapeutic agent. Exemplary carriers include saline, buffered saline, dextrose, water, glycerol, ethanol, and combinations thereof. For drugs administered orally, pharmaceutically acceptable earners include, but are not limited to pharmaceutical acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while com starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

By “proliferative disease” or “cancer” as used herein is meant, a disease, condition, trait, genotype or phenotype characterized by unregulated cell growth or replication as is known in the art; including colorectal cancer, as well as, for example, I Acute Lymphoblastic Leukemia (ALL); Acute Myeloid Leukemia (AML); Adolescents, Cancer in; Adrenocortical Carcinoma; Childhood Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Gastrointestinal Carcinoid Tumors; Astrocytomas, Childhood (Brain Cancer); Atypical Teratoid/Rhabdoid Tumor, Childhood, Central Nervous System (Brain Cancer); Basal Cell Carcinoma of the Skin; Bile Duct Cancer; Bladder Cancer; Childhood Bladder Cancer; Bone Cancer (includes Ewing Sarcoma and Osteosarcoma and Malignant Fibrous Histiocytoma); Brain Tumors; Breast Cancer; Bronchial Tumors (Lung Cancer); Burkitt Lymphoma-Non-Hodgkin Lymphoma-; Carcinoid Tumor (Gastrointestinal); Childhood Carcinoid Tumors; Carcinoma of Unknown Primary; Childhood Carcinoma of Unknown Primary; Cardiac (Heart) Tumors, Childhood; Central Nervous System; Atypical Teratoid/Rhabdoid Tumor, Childhood (Brain Cancer); Medulloblastoma and Other CNS Embryonal Tumors, Childhood (Brain Cancer); Germ Cell Tumor, Childhood (Brain Cancer); Primary CNS Lymphoma; Cervical Cancer; Childhood Cervical Cancer; Childhood Cancers; Cancers of Childhood, Unusual; Cholangiocarcinoma-Bile Duct Cancer-; Chordoma, Childhood; Chronic Lymphocytic Leukemia (CLL); Chronic Myelogenous Leukemia (CML); Chronic Myeloproliferative Neoplasms; Colorectal Cancer; Childhood Colorectal Cancer; Craniopharyngioma, Childhood (Brain Cancer); Cutaneous T-Cell Lymphoma (Mycosis Fungoides and Sezary Syndrome); Ductal Carcinoma In Situ (DCIS)—Breast Cancer—; Embryonal Tumors, Medulloblastoma and Other Central Nervous System, Childhood (Brain Cancer); Endometrial Cancer (Uterine Cancer); Ependymoma, Childhood (Brain Cancer); Esophageal Cancer; Esthesioneuroblastoma (Head and Neck Cancer); Ewing Sarcoma (Bone Cancer); Extracranial Germ Cell Tumor, Childhood; Extragonadal Germ Cell Tumor; Eye Cancer; Childhood Intraocular Melanoma; Intraocular Melanoma; Retinoblastoma; Fallopian Tube Cancer; Fibrous Histiocytoma of Bone, Malignant, and Osteosarcoma; Gallbladder Cancer; Gastric (Stomach) Cancer; Childhood Gastric (Stomach) Cancer; Gastrointestinal Carcinoid Tumor; Gastrointestinal Stromal Tumors (GIST) (Soft Tissue Sarcoma); Childhood Gastrointestinal Stromal Tumors; Germ Cell Tumors; Childhood Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors, Childhood; Hepatocellular (Liver) Cancer; Histiocytosis, Langerhans Cell; Hodgkin Lymphoma; Hypopharyngeal Cancer (Head and Neck Cancer); Intraocular Melanoma; Childhood Intraocular Melanoma; Islet Cell Tumors, Pancreatic Neuroendocrine Tumors; Kaposi Sarcoma (Soft Tissue Sarcoma); Kidney (Renal Cell) Cancer; Langerhans Cell Histiocytosis; Laryngeal Cancer (Head and Neck Cancer); Leukemia; Lip and Oral Cavity Cancer (Head and Neck Cancer); Liver Cancer; Lung Cancer (Non-Small Cell, Small Cell, Pleuropulmonary Blastoma, and Tracheobronchial Tumor); Lymphoma; Male Breast Cancer; Malignant Fibrous Histiocytoma of Bone and Osteosarcoma; Melanoma; Childhood Melanoma; Melanoma, Intraocular (Eye); Childhood Intraocular Melanoma; Merkel Cell Carcinoma (Skin Cancer); Mesothelioma, Malignant; Metastatic Cancer; Metastatic Squamous Neck Cancer with Occult Primary (Head and Neck Cancer); Midline Tract Carcinoma With NUT Gene Changes; Mouth Cancer (Head and Neck Cancer); Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms, Chronic; Nasal Cavity and Paranasal Sinus Cancer (Head and Neck Cancer); Nasopharyngeal Cancer (Head and Neck Cancer); Neuroblastoma; Non-Hodgkin Lymphoma; Non-Small Cell Lung Cancer; Oral Cancer, Lip and Oral Cavity Cancer and Oropharyngeal Cancer (Head and Neck Cancer); Osteosarcoma and Malignant Fibrous Histiocytoma of Bone; Ovarian Cancer; Childhood Ovarian Cancer; Pancreatic Cancer; Childhood Pancreatic Cancer; Pancreatic Neuroendocrine Tumors (Islet Cell Tumors); Papillomatosis (Childhood Laryngeal); Paraganglioma; Childhood Paraganglioma; Paranasal Sinus and Nasal Cavity Cancer (Head and Neck Cancer); Parathyroid Cancer; Penile Cancer; Pharyngeal Cancer (Head and Neck Cancer); Pheochromocytoma; Childhood Pheochromocytoma; Pituitary Tumor; Plasma Cell Neoplasm/Multiple Myeloma; Pleuropulmonary Blastoma (Lung Cancer); Pregnancy and Breast Cancer; Primary Central Nervous System (CNS) Lymphoma; Primary Peritoneal Cancer; Prostate Cancer; Rectal Cancer; Recurrent Cancer; Renal Cell (Kidney) Cancer; Retinoblastoma; Rhabdomyosarcoma, Childhood (Soft Tissue Sarcoma); Salivary Gland Cancer (Head and Neck Cancer); Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Soft Tissue Sarcoma; Uterine Sarcoma; Sezary Syndrome (Lymphoma); Skin Cancer; Childhood Skin Cancer; Small Cell Lung Cancer; Small Intestine Cancer; Soft Tissue Sarcoma; Squamous Cell Carcinoma of the Skin; Squamous Neck Cancer with Occult Primary, Metastatic (Head and Neck Cancer); Stomach (Gastric) Cancer; Childhood Stomach (Gastric) Cancer; T-Cell Lymphoma, Cutaneous-Lymphoma (Mycosis Fungoides and Sezary Syndrome); Testicular Cancer; Childhood Testicular Cancer; Throat Cancer (Head and Neck Cancer); Nasopharyngeal Cancer; Oropharyngeal Cancer; Hypopharyngeal Cancer; Thymoma and Thymic Carcinoma; Thyroid Cancer; Tracheobronchial Tumors (Lung Cancer); Transitional Cell Cancer of the Renal Pelvis and Ureter (Kidney (Renal Cell) Cancer); Unknown Primary, Carcinoma of; Childhood Cancer of Unknown Primary; Ureter and Renal Pelvis, Transitional Cell Cancer (Kidney (Renal Cell) Cancer; Urethral Cancer; Uterine Cancer, Endometrial; Uterine Sarcoma; Vaginal Cancer; Childhood Vaginal Cancer; Vascular Tumors (Soft Tissue Sarcoma); Vulvar Cancer; Wilms Tumor and Other Childhood Kidney Tumors; multidrug resistant cancers; and proliferative diseases and conditions, such as neovascularization associated with tumor angiogenesis and other cancer or proliferative disease, condition, trait, genotype or phenotype that can respond to the modulation of its environment, alone or in combination with other therapies.

The term “sample” as used herein refers to a biological sample obtained for the purpose of evaluation in vitro. With regard to the methods disclosed herein, the sample or patient sample preferably may comprise any fluid or tissue. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis).

Preferred samples are whole blood, serum, plasma, or urine. A sample can also be a partially purified fraction of a tissue or bodily fluid.

The term “Sepsis” generally refers to a syndrome of systemic immune response to an infection or to microbial pathogenic components (liv). Diabetes mellitus, lymphoproliferative disease, hepatic cirrhosis, respiratory illnesses and diseases (like Acute respiratory distress syndrome (ARDS), asthma, Chronic Obstructive Pulmonary Disease (COPD), chronic bronchitis, emphysema, lung cancer, cystic fibrosis/bronchiectasis, pneumonia, pleural effusion, etc), extensive burning, severe trauma, use of intravenous or vesicular catheters, prosthesis and treatments with immunosuppressive medicines or intravenous drugs are frequent causes that contribute to acquisition of infections resulting in Sepsis.

Stimulus prompting Sepsis can be exogenous (i.e. infectious) or endogenous (i.e. severe trauma) and all of them act as alarm signal for the immune system and in general of the system in charge of keeping homeostasis. However, during Sepsis this system become deregulated causing multiple organic damage.

The first period of Sepsis is characterized by the accumulation of oxygen and nitrogen reactive forms. Some symptoms corresponding to this period are increment of the cardiac frequency, tachycardia, fever and neutrophilia. Following, elevation of proinflammatory cytokines and chemokines in plasma as well as migration of polymorphonuclear leukocytes, monocytes and lymphocytes towards tissues occurs. Own to this dramatic presentation, the prevalent definition for Sepsis has been for a long time that Sepsis is, basically, an uncontrolled inflammatory response. However, a number of recent and rigorous observations have conducted to a redefinition of Sepsis (Iv), bringing about the idea that in Sepsis there exist successive pro-inflammatory and anti-inflammatory (immunosuppressive) periods. Even though, same patients die during the first pro-inflammatory period because of a Septic Shock or meningococcemia currently most of the patients survive this period (Ivi, lvii) The great majority of death occur during the immunosuppressive period, which in general starts between the second and third day of Sepsis and could last several weeks. In spite of antibiotic treatment and strong control medical actions many patients cannot eradicate the infection and may also get secondary nococomial infections (lviii, lix).

Patients with sepsis are in high risk of morbidity and mortality mainly because of the multiple organ dysfunction and treatment complications.

The terms “subject”, “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates. Patients in need of therapy comprise those at risk of developing a certain condition, disease or disorder (e.g. due to genetic, environmental or physical attributes, such as for example, obesity)—Patients in need of therapy also include those afflicted with a condition, disease or disorder. The diseases or disorders comprise, for example: autoimmune diseases, cancer, inflammatory diseases, neurological diseases or disorders, neuroinflammatory diseases or disorders, cardiovascular disease, obesity, diseases or disorders caused by infectious agents such as, for example, viruses, bacteria, fungi, prions, or parasites.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

“Treating” or “treatment” covers the treatment of a disease-state in a mammal, and includes: (a.) preventing the disease-state from occurring in a mammal, in particular, when such mammal is predisposed to the disease-state but has not yet been diagnosed as having it; (b) inhibiting the disease-state, e.g., arresting it development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc.).

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The practice of the present invention employs, unless otherwise indicated, conventional techniques of chemistry, molecular biology, microbiology, recombinant DNA, genetics, immunology, cell biology, ceil culture and transgenic biology, which are within the skill of the art. See, e.g., Maniatis etal., 1982, Molecular Cloning (Cold Spring Harbor Laboratory Press, Cold Spring Harbor, N.Y.); Sambrook etal, 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al, 1992), Current Protocols in Molecular Biology (John Wiley & Sons, including periodic updates); Glover, 1985, DNA Cloning (1RL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984): Transcription And Translation (B. D. Hames & S. J. Higgins eds. 1984): Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds. . . . Academic Press, London, 1987); Handbook Of Experimental Immunology, Volumes I-IV (D. M. Weir and C. C. Blackwell, eds., 1986); Riott, Essential Immunology7, 6th Edition, Blackweli Scientific Publications, Oxford, 1988; Hogan etal., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

### BRIEF DESCRIPTION OF THE DRAWINGS

FIG. 1: Developed formula of ODN CpG 2006, main representative molecule of GpG ONs and ODN IMT504, main representative molecule of PyNTTTTGT ONs.

FIG. 2: 3D view of ODN CpG 2006 (A.), ODN IMT504 (B) and 24mer phosphorotioate NA backbone (C), showing with bigger atoms size the active sites (CpG2006 and IMT504) and the Sulphur atoms in phosphorotioate backbone. FIGS. 2.A and 2.B highlight pharmacophore domain of each ON and 2.C. the dianóforo domain of both.

FIG. 3: Developed formula of different carbon chains used as molecular spacers.

FIG. 4: Developed formula of IMT504 Phosphodiester, IMT504 Phosphotioated and respective C3 amino derivatives.

FIG. 5. Hyaluronic Acid schematic formula showing Active Sites

FIG. 6: Hyaluronic Acid/ODN coupling reaction scheme via carbodiimide reaction

FIG. 7: Chromatographic purification of HA(100)-ON1 form the reaction mixture (A). and analytical HPLC of the purified HA(100)-ON1 peak.

FIG. 8: Polyethylene Glycol schematic formula showing Active Sites

FIG. 9: Polyethylene Glycol/ODN coupling reaction scheme via carbodiimide reaction

FIG. 10: Acyl chloride functionalized polyethylene Glycol/ODN coupling reaction scheme

FIG. 11: Anhydride functionalized polyethylene Glycol/ODN coupling reaction scheme

FIG. 12: Aldehyde functionalized polyethylene Glycol/ODN coupling reaction scheme

FIG. 13: Isothiocyanate functionalized polyethylene Glycol/ODN coupling reaction scheme

FIG. 14: Chromatographic purification of PEG(40)-ON6 form the reaction mixture (A). and analytical HPLC of the purified PEG(40)-ON6 peak.

FIG. 15: Schematic formula of some example of Lipids: Palmitic Acid, Stearic Acid, Oleic Acid, Linoleic Acid, Alpha Linolenic Acid and Docosahexanoic Acid

FIG. 16: Palmitic Acid/ODN coupling reaction scheme via carbodiimide reaction

FIG. 17: Docosahexaenoic/ODN coupling reaction scheme via acyl chloride formation

FIG. 18: Chromatographic purification of Myr-ON6 form the reaction mixture (A). and analytical HPLC of the purified Myr-ON6 peak.

FIG. 19: Graph showing the evolution of mechanical allodynia before and after (arrows) IMT504 (5 daily consecutive shots of 20 mg/Kg) and LLONCs Palm-ON1 and HA(100)-ON1 (only one shot of 0.4 mg/Kg) administration.

FIGS. 20 and 21 demonstrate the comparative effects of LLONC HA(100)-ON1 regarding to the standard PyTTTGT ON (IMT504) on mature oligodendrocytes in the Corpus Callosum (CC) of CPZ-demyelinated rats. FIG. 20: Representative images of Platelet-derived growth factor receptor (+) (PDGFR (+)) cells—as a marker for neural stem cells—and myelin-associated glycoprotein (MAG) immunohistochemistry in the CC of control (Control+SS) and CPZ treated animals 7 days after injection with saline solution (CPZ+SS, ×5 daily), IMT504 (20 mg/kg body weight, ×5 daily, CPZ+IMT504), LLONC HA(100)-ON1 (0.4 mg/kg body weight, just one injection, CPZ+HA(100)-ON1); cell nuclei visualized with Hoechst. Magnified images show MAG positive cells. FIG. 21: Quantification of MAG positive cells. Results are expressed as positive cells by area.

FIGS. 22 and 23 are graphs showing the comparative effects of HA(100)-ON1 vs. IMT504 on the inflammatory response in the Corpus Callosum (CC) of CPZ-demyelinated rats. Quantification of Allograft Inflammatory Factor I (IBA.1) (FIG. 22) and the inflammatory marker CD68 (FIG. 23) positive microglial cells by immunohistochemistry in the CC of CPZ and control animals 7 days before the CPZ withdrawal. Results are expressed as positive cells by area.

FIG. 24 shows the comparative effects of LLONCc Myr-ON1, Myr-ON6, PEG(40)-ON1 and PEG(40)-ON1 and the standard PyTTTGT ON (IMT504) on mature oligodendrocytes in the Corpus Callosum (CC) of CPZ-demyelinated rats. Figure shows representative images of Platelet-derived growth factor receptor (+) cells—as a marker for neural stem cells- and myelin-associated glycoprotein (MAG) immunohistochemistry, Allograft Inflammatory Factor 1 (IBA.1) and the inflammatory marker CD68 positive microglial cells, in the CC of control (Control+SS) and CPZ treated animals 7 days after injection with saline solution (CPZ+SS, ×5 daily), IMT504 (20 mg/kg body weight, ×5 daily (CPZ-IMT504) and Myr-ON1 (CPZ-ODN1-Myr), Myr-ON6 (CPZ-ODN6-Myr), PEG(40)-ON1 (CPZ-ODN1-PEG) and PEG(40)-ON6 (CPZ-ODN6-PEG) (0.4 mg/kg body weight, just one injection, CPZ+HA(100)-ON1); cell nuclei visualized with Hoechst. Magnified images show MAG positive cells. FIG. 21: Quantification of PDGFR, MAG, AND CD68 positive microglial cells. Results are expressed as positive cells by area.

FIG. 25. Secondary effects of cytostatic protection. Balb/c mice were randomly divided into seven groups (n=5; N=35), namely, Control CPt(−) group which was injected (sc) with 200 □l of phosphate buffered saline (PBS); Control CPt(+) group which was injected (sc) with 200 □l of 0.05 mM phosphate buffered saline (PBS) pH 7.4. IMT504 group which was injected (sc) with 200 □l of IMT504 (20 mg/ml) in PBS (total dose of 20 mg/kg animal weight) every day during the first 5 consecutive days. PEG(20)-ON1 200 group which was injected (sc) with 200 □l of PEG(20)-ON1 in PBS (200 □g/kg animal weight) in only one shot. PEG(20)-ON1 20 group which was injected (sc) with 200 □l of PEG(20)-ON1 in PBS (20 g/kg animal weight) in only one shot. Palm-ON1 200 group which was injected (sc) with 200 □l of Palm-ON1 in PBS (200 □g/kg animal weight) also in only one shot and Palm-ON1 20 group which was injected (sc) with 200 □l of Palm-ON1 in PBS (20 □g/kg animal weight) also in only one shot. Two days after, all animals, except those belonging to the group Control CPt(−) have received an intraperitoneal injection of cisplatin (20 mg kg-1). After the treatment with cisplatin each dead animal was autopsied immediately and the survived ones were sacrificed, and autopsied 14 days after Cisplatin treatment. Spleen and kidneys of any animal were collected and were kept in 4% paraformaldehyde for 24 h and then embedded in paraffin wax. Tissues were sectioned and stained with Masson's trichrome and hematoxylin and eosin (H&E) stains before examination under a light microscope.

Survival curve of animals were drawn (FIG. 25A) and data were analyzed by a Kaplan-Meier test. Macroscopic comparison of spleen (FIG. 25.B) and histological comparison of spleen (FIG. 25.C) and kidney ((FIG. 25.D) are also shown.

FIG. 26 shows the comparative effects of different adjuvants of recombinant gonadotropin-releasing hormone (GnRH) as a contraceptive vaccine.

FIG. 26.A. Kinetics of the testosterone level (triangles) of rat males after immunization with PBS (None), GnRH+Chitosan, GnRH+IMT504 and GnRH+HA(100)-ON1 in 3 different amounts (50, 5 and 0.05 ug of HA(100)-ON1/dose).

FIG. 26.B.Testis volume (bars) and the testosterone level (triangles) of rat males 16 weeks after treated with PBS (None), GnRH+Chitosan, GnRH+HA(100)-ON1 or GnRH+IMT504

FIG. 27 shows the comparative effects of different PyNTTTTGT adjuvants. Young male Wistar rats, 4-week-old, were taken for immunization and assigned to various groups randomly on equitable basis of body weights. Vero cell RABV vaccine (VeroRab™, Sanofi-Pasteur) containing ≥2.5 IU per dose was used for immunization of rats.

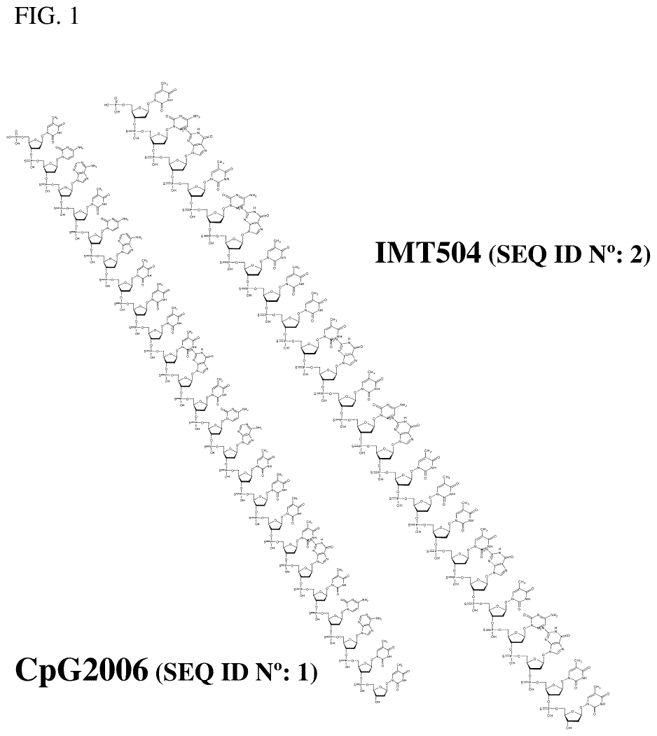

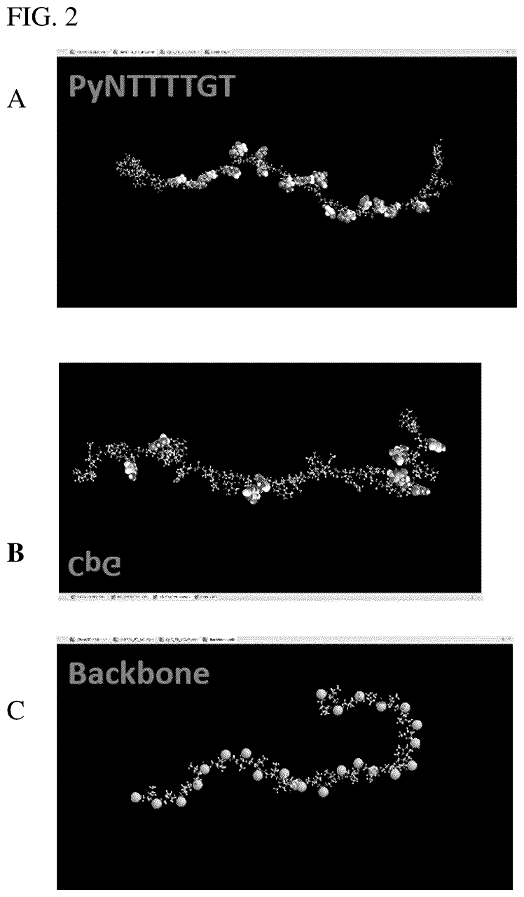

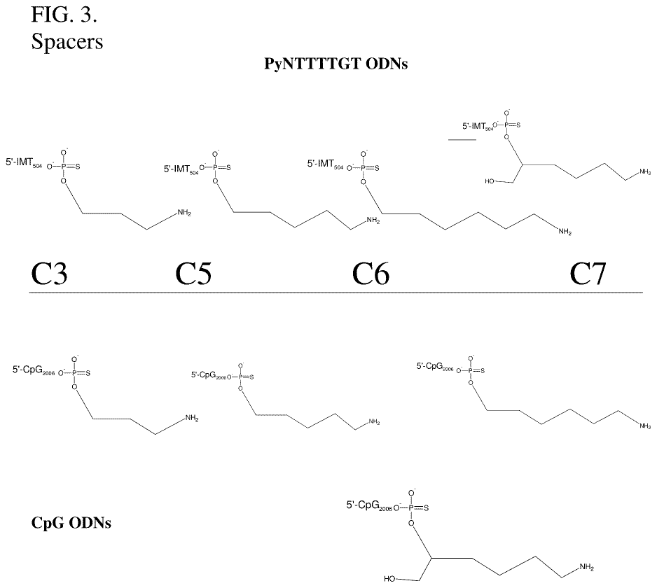

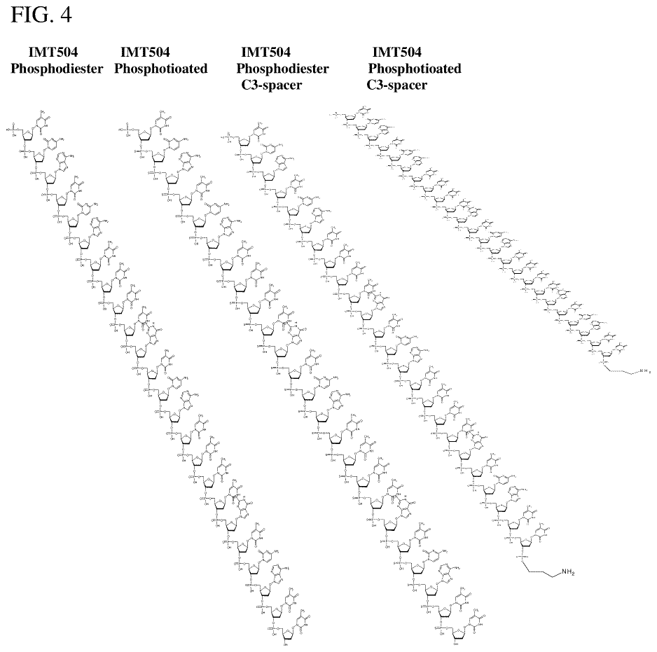

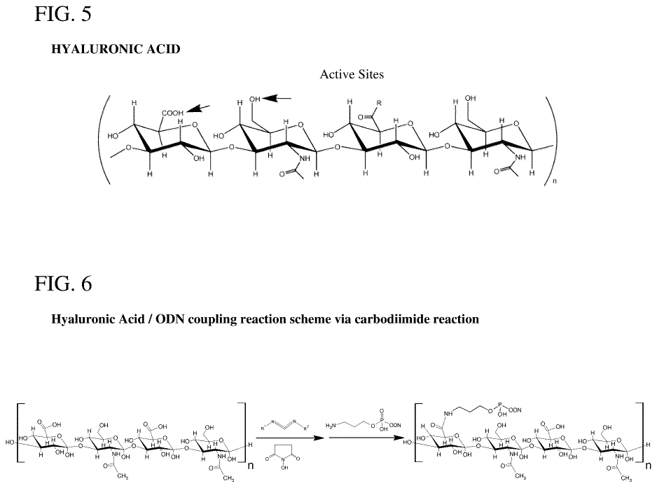

Each rat received a single subcutaneous injection containing 50 μl of a 1/125 dilution of the original vaccine. Control groups received the vaccine alone (control) and experimental groups received vaccine with the addition of 50 μg (IMT504 50) or 10 μg (IMT504 10) of ODN IMT504. Or 5 μg (ODN1-Palm 5) or 1 μg (ODN1-Palm 1) or 0.2 μg (ODN1-Palm 0.2) of Palm-ON1. Or 5 μg (ODN1-PEG 5) or 1 μg (ODN1-PEG 1) or 0.2 μg (ODN1-PEG 0.2) of PEG(20)-ON1

Each group of rats was injected subcutaneously (s.c.) with the corresponding composition and then, the same composition booster injections were given 2 and 4 weeks after the first shot.

At times 0, 14, 28 and 42 days, the rats were bled (collected by retro-orbital puncture) and sera separated and stored at −20° C. until assayed for specific total IgG antibody determination by ELISA. Optical Density (OD) was measured at 490 nm.

FIG. 28 shows the comparative effects of different CpG adjuvants. Young male Wistar rats, 4-week-old, were taken for immunization and assigned to various groups randomly on equitable basis of body weights. Vero cell RABV vaccine (VeroRab™, Sanofi-Pasteur) containing ≥2.5 IU per dose was used for immunization of rats.

Each rat received a single subcutaneous injection containing 50 μl of a 1/125 dilution of the original vaccine. Control groups received the vaccine alone (control) and experimental groups received vaccine with the addition of 50 μg (CPG2006 50) or 10 μg (CPG2006 10) of ODN CPG2006. Or 5 μg (ODN5-Palm 5) or 1 μg (ODN1-Palm 1) or 0.2 μg (ODN1-Palm 0.2) of Palm-ON5. Or 5 μg (ODN5-PEG 5) or 1 μg (ODN5-PEG 1) or 0.2 μg (ODN5-PEG 0.2) of PEG(20)-ON5.

Each group of rats was injected subcutaneously (s.c.) with the corresponding composition and then, the same composition booster injections were given 2 and 4 weeks after the first shot.

At times 0, 14, 28 and 42 days, the rats were bled (collected by retro-orbital puncture) and sera separated and stored at −20° C. until assayed for specific total IgG antibody determination by ELISA. Optical Density (OD) was measured at 490 nm.

FIG. 29 shows the comparative effects of LLONC HA(1000)-ON1 vs. IMT504 and positive control in an animal model of Chronic Lymphocytic Leukemia. Tumor induced animals were untreated (Control) or treated with IMT504 (20 mg/ml) in PBS (total daily dose of 20 mg/kg animal weight) every day during the first 5 consecutive days (total dose 100 mg/kg) or with 4.5 mg/kg animal weight of HA(1000)-ON1 (injected in only one shot; total dose 4.5 mg/Kg).

Animals were sacrificed at day 13 and cells recovered from peripheral blood, peritoneal cavity and spleen compartment samples and were processed and quantified by mean of cytometric analysis, measuring CD19+, CD5+ B cells. The cytometric results were summarized in a bar figure.

FIG. 30 shows the comparative effects of LLONC Myr-ON1 and Myr-ON6 vs. IMT504 and positive control in an animal model of Chronic Lymphocytic Leukemia. Tumor induced animals were untreated (Control) or treated with IMT504 (20 mg/ml) in PBS (total daily dose of 20 mg/kg animal weight) every day during the first 5 consecutive days (total dose 100 mg/kg) or with 2 mg/kg animal weight of Myr-ON1 or with 2 mg/kg animal weight of Myr-ON6 (injected in only one shot; total dose 2 mg/Kg).

Animals were sacrificed at day 11 and cells recovered from peritoneal cavity were processed and quantified by mean of cytometric analysis, measuring CD19+, CD5+ B cells. The cytometric results were summarized in a bar figure.

FIG. 31 shows the comparative effects of LLONC PEG(40)-ON1 and PEG(40)-ON6 vs. IMT504 and positive control in an animal model of Chronic Lymphocytic Leukemia. Tumor induced animals were untreated (Control) or treated with IMT504 (20 mg/ml) in PBS (total daily dose of 20 mg/kg animal weight) every day during the first 5 consecutive days (total dose 100 mg/kg) or with 1 mg/kg animal weight of PEG(40)-ON1 or with 1 mg/kg animal weight of PEG(40)-ON6 (injected in only one shot; total dose 1 mg/Kg).

Animals were sacrificed at day 11 and cells recovered from peritoneal cavity were processed and quantified by mean of cytometric analysis, measuring CD19+, CD5+ B cells. The cytometric results were summarized in a bar figure.

FIG. 32 shows the comparative effects of CpG LLONCs PEG(20)-ON5 and Palm-ON5 vs. IMT504 and CpG2006 and positive control in an animal model of Chronic Lymphocytic Leukemia. Tumor induced animals were untreated (Control) or treated with IMT504 (20 mg/ml) in PBS (total daily dose of 20 mg/kg animal weight) every day during the first 5 consecutive days (total dose 100 mg/kg) or CpG2006 (20 mg/ml) in PBS (total daily dose of 20 mg/kg animal weight) every day during the first 5 consecutive days (total dose 100 mg/kg) or with 1 mg/kg animal weight of PEG(20)-ON5 or with 1 mg/kg animal weight of PEG(20)-ON5 (injected in only one shot; total dose 1 mg/Kg).

Animals were sacrificed at day 11 and cells recovered from peritoneal cavity were processed and quantified by mean of cytometric analysis, measuring CD19+, CD5+ B cells. The cytometric results were summarized in a bar figure.

FIG. 33 shows the comparative effects of LLONC HA(1000)-ON1 vs. IMT504 on the melanoma growth. Tumor induced animals (injecting B16-F10 melanoma cells) were untreated (Control) or treated with IMT504 (20 mg/ml) in PBS (total daily dose of 20 mg/kg animal weight) every day during the first 5 consecutive days or with HA(1000)-ON1 (injected with 3.5 mg/kg animal weight in only one shot).

Mice were observed for tumor growth and a caliper was used to measure perpendicular tumor diameters at time 9, 12, 14 and 16 days after the first (or only) shot of the treatment and the total tumor volume was calculated.

FIG. 34 shows the comparative effects of LLONC HA(1000)-ON1 vs. IMT504 on the breast cancer growth.

Tumor induced animals (4T1 murine mammary carcinoma cells) were untreated (Control) or treated with IMT504 (20 mg/ml) in PBS (total daily dose of 20 mg/kg animal weight) every day during the first 5 consecutive days or with HA(1000)-ON1 (injected with 3.5 mg/kg animal weight in only one shot). Mice were observed for tumor growth and a caliper was used to measure perpendicular tumor diameters at time 9, 12, 14 and 16 days after the first (or only) shot of the treatment and the total tumor volume was calculated.

FIG. 35 shows the comparative effects of LLONC HA(100)-ON1 and HA(1000)-ON1 vs. IMT504 and positive control in the treatment on the sepsis in mouse model induced by LPS. It shows the Kaplan-Meier analysis of survival mice in a sepsis model.

FIG. 36 shows the comparative effects of LLONC HA(1000)-ON1 vs. IMT504 and positive control in the evolution on the serum titer of IL6, as a marker of the cytokine storm induced in mouse by LPS.

FIG. 37 Effect of Palm-ON1 on Colitis mouse model induced by TNBS. Gut was removed from sacrificed animals, and the colon was cut close to the ileocecal valve. Distal colon sections (2 cm) were cut and histologically analyzed estimating the histological activity index (HAI).

### DETAILED DESCRIPTION

Immunostimulatory oligonucleotides (IS-ONs) are a class of synthetic or naturally occurring short sequences of DNA or RNA that have gained significant attention in the field of immunology and medical research. These molecules are designed to harness the power of the immune system by stimulating various components of the innate and adaptive immune responses. IS-ONs serve as potent immunomodulators, triggering a cascade of immune reactions that can be harnessed for therapeutic and research purposes. Their versatile applications range from enhancing vaccine efficacy to treating autoimmune diseases, and from cancer immunotherapy to investigating the intricacies of immune function.

The stimulation of the immune system by synthetic CpG ODN via TLR9 leads to a variety of directed effects linking innate to adaptive immune responses. TLR9 agonists can be used as highly effective targeted immune modulators with broad potential applications as vaccine adjuvants, and as stand-alone therapy or in combination with other therapies in cancer, infectious diseases or asthma and allergy. Animal models and ongoing phase I to phase III clinical trials suggest that CpG ODN have anti-tumor activities and are effective vaccine adjuvants in a variety of disease indications (Ix).

Some CpG ODNs induce highly purified human adult B cells to proliferate, produce IgM, IgG and IgA and increase cell surface expression of CD86 (a marker of B cell activation) and CD25 (the IL-2 receptor). Maximal human B cell stimulation (cellular proliferation, CD80 and CD86 expression, immunoglobulin production and IL-6 secretion) is achieved with ODNs that possess a nuclease-resistant phosphorothioate-modified backbone with one or more CpG motifs and no polyG motif. The CpG ODNs that induce a Th-1 response and also stimulate B cells potently belong to the B class (also known as K type), while the A class (also known as D type) are potent in activating NK cells and human plasmacytoid dendritic cells to secrete interferon-α. A third class of CpG is known as the C class, which combines the properties of both A and B classes by being able to stimulate B cell and NK cell activation and IFN-α production. The B-class CpG ODNs enhance the ability of dendritic cells to produce IL-12 and help polarize T cell responses in the TH1 direction. Several CpG ODNs are in clinical trials to enhance vaccine responses to infective agents and cancer cells (lxi).

On the other hand, the potential therapeutic effect of a family of homeostatic single strand phosphorothioated oligodeoxynucleotides (ON) with the active motif represented by the sequence corresponding to the general formula PyNTTTTGT in which Py is C or T, and N is A, T, C, or G was demonstrated in several animal models (references). These molecules have potent effects for the human and primate innate and adaptive immune systems. They directly activates B cells to produce beneficial cytokines, promotes antibody production and maturation, expands antibody diversity, and potentiates the capacity of B cells to serve as antigen-presenting cells (lxii). PyNTTTTGT ONs can also activate plasmacytoid dendritic cells, which are important components of the antigen-presenting cells in the human immune system as well as regulators of the immune response (15 y lxiii). Moreover, these homeostatic ONs activates CD4 and CD8 T cells and natural killer cells (NK cells and NKT cells) to promote cellular immune functions with activities against specific forms of cancer and virusinfected cells (lxiv). In addition, preclinical studies indicate an excellent safety profile (lxv, lxvi, lxvii). Stimulation of these cells results in secretion of cytokines such as IL6, IL10, GM-CSF, IFN and tumor necrosis factor-□, secretion of immunoglobulins, expression of cell surface proteins such as CD25, CD40, CD80, and CD86, and major histocompatibility complex classes I and II. Also, incubation of lymphocytes B with PyNTTTTGT ONs results in protection of these cells from spontaneous apoptosis and expansion of at least a subset of these cells [lxviii, 16]. B cells produce cytokines in response to their environment.

The immune homeostatic response of animals to aggression (infections, traumas, tumor transformation, radiation etc) is based on an intrincate network of cells and chemical messengers. Abnormally high inflammation immediately after aggression or abnormally prolonged pro-inflammatory stimulus bringing about chronic inflammation are associated to life threatening or severe disabling diseases. Mesenchymal stem cells (MSC) transplant has demonstrated to be an effective therapy in preclinical studies representing a vast amount of inflammatory conditions. As alternative to MSC transplant, the PyNTTTTGT ONs with capacity to boost human MSC expansion and/or activation in vivo may also be effective. Regarding to this, the mentioned homeostatic and immunomodulatory ONs induce in vivo expansion of MSC resulting in a marked improving of animals suffering Neural Pain, Osteoporosis, Diabetes and Sepsis (lxix).

Participation of MSCs in the pathway stimulated by PyNTTTTGT ONs migth modulate the inflammatory process, thereby stimulating the switch from the pro-inflammatory to the anti-inflammatory reconstructive stage of the immune response (lxx).

Conjugated Nucleic Acid: In all tested cases (neuropathic pain, demyelinization, osteoporosis, diabetes and sepsis), the main inconvenience of these new therapeutic molecules is the requirement of daily injections of the molecule over 5 successive days. In an attempt to improve this issue, the PyNTTTTGT ONs were conjugated with different recognized long-lasting organic molecules. Single stranded nucleic acids to be conjugated can be chemically synthesized, either as a single nucleic acid molecule (e.g., using automated DNA synthesis in 3′ to 5′ direction using phosphoramidite technology) or as a series of oligonucleotides. Necessary chemical modifications on the ONs for allowing the conjugation reaction can be added before, during or after the synthesis.

Binding entity for conjugation: In certain embodiments, the selected nucleic acid may be combined directly with a selected long-lasting organic molecule. In other embodiments, the selected nucleic acid may have a minor modification (—NH2 or —COOH addition) for allowing the covalent combination with a selected long-lasting organic molecule. However, the use of single spacers (e.g. C3, C5, C7 hydrocarbon chains) or much more complex ones (peptides, terpenes, receptors, hormones, etc) are also embodied herein. The ONs can be eventually modified in just one position or in several locations along the sequence. In case than more than one points of binding were selected, both, the binding entities and the long-lasting organic molecules selected may be all the same or different.

Long-lasting organic molecules: In certain embodiments, the selected long-lasting organic molecule of the invention involves moieties include but are not limited to lipid moieties such as a cholesterol moiety, a cholesteryl moiety (¹¹²), cholic acid (¹¹³), a thioether, e.g., hexyl-S-tritylthiol (^{114, 115}), a thiocholesterol (¹¹⁶), an aliphatic chain, e.g., dodecandiol or undecyl residues (^{117, 118, 119}), a phospholipid, e.g., di-hexadecyl-rac-giycerol or triethylammonium 1,2-di-0-hexadecyi-rac-glycero-3-H-phosphonate (^{120, 121}), a polyamine or a polyethylene glycol chain (¹²²), or adamantane acetic acid (¹²³) or complex polysaccharides, like Hyaluronic Acid (^{124, 125}).

The ONs can be conjugated directly or through a binding entity. It is not necessary for all positions in a given nucleic acid sequence to be uniformly modified, and in fact more than one of the aforementioned modifications may be incorporated in a single nucleic acid sequence or even at within a single nucleoside within a nucleic acid sequence.

Nucleic Acid Sequences: In certain embodiments, a pharmaceutical composition comprises a therapeutically effective amount of a LLONC. Following, the term “percent sequence identity” refers to the degree of identity between any given query sequence and a subject sequence. In certain embodiments, a pharmaceutical composition comprises a therapeutically effective amount of a LLONC, where the oligonucleotide component has at least a 50% sequence identity to SEQ ID N^{o}: 1. In certain embodiments, a pharmaceutical composition comprises a therapeutically effective amount of a LLONC, where the oligonucleotide component has at least a: 60%, 70%, 75%, 80%, 85%, 90%, 91%, 92%, 93%, 94%, 95%, 96%, 97%, 98%, 99%, or at least a 99.9% sequence identity to SEQ ID N^{o}: 1. In all cases, the sequence should conserve at least one unmethylated CpG pair.

In certain embodiments, a pharmaceutical composition comprises a therapeutically effective amount of a LLONC, where the oligonucleotide component has at least a 50% sequence identity to SEQ ID N^{o}: 2. In certain embodiments, a pharmaceutical composition comprises a therapeutically effective amount of a LLONC, where the oligonucleotide component has at least a: 60%, 70%, 75%, 80%, 85%, 90%, 91%, 92%, 93%, 94%, 95%, 96%, 97%, 98%, 99%, or at least a 99.9% sequence identity to SEQ ID N^{o}: 2. In certain embodiments, a pharmaceutical composition comprises a therapeutically effective amount of a LLONC, where the oligonucleotide component has at least a: 90%, 91%, 92%, 93%, 94%, 95%, 96%, 97%, 98%, 99%, or at least a 99.9% sequence identity to the specific antisense ON. In all cases, the sequence should conserve at least one active site with the sequence PyNTTTTGT.

Modified Nucleic Acid Sequences: In certain embodiments, the nucleic acid sequence of the ON in the LLONC may be modified or derived from a native nucleic acid sequence, for example, by introduction of mutations, deletions, substitutions, modification of nucleobases, backbones and the like. Examples of some modified nucleic acid sequences envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, modified oligonucleotides comprise those with phosphorothioate backbones and those with heteroatom backbones, wherein the native phosphodiester backbone is represented as O—P—O—CH). The amide backbones disclosed by De Mesmaeker et al. (^{lxxi}) are also embodied herein. In some embodiments, the nucleic acid sequences having morpholino backbone structures (lxxii), peptide nucleic acid (PNA) backbone wherein the phosphodiester backbone of the oligonucleotide is replaced with a poliyamide backbone, the nucleobases being bound directly or indirectly to the aza nitrogen atoms of the poliyamide backbone (lxxiii). The nucleic acid sequences may also comprise one or more substituted sugar moieties. The nucleic acid sequences may also have sugar mimetics such as cyclobutyl in place of the pentofuranosyl group.

The nucleic acid sequence of the ON of the LLONC may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(m.ethylamino) adenine, 2-(imidazolylalkyl) adenine, 2-(aminoalklyamino) adenine or other heterosubstituted alkyladenines, 2-thiouracil, 2-thiothymine, 5-bromouracil, 5-hydroxymethyluracil, 8-azaguanine, 7-deazaguanine, N⁶ (6 aminohexyl) adenine and 2,6-diaminopurine. A “universal” base known in the art, e.g., inosine may be included.

### Methods and Uses Thereof

The invention provides a method for preparing LLONCs . . . .

One method comprises LLONCs, were the ON selected for conjugation has the sequence 5′-TCGTCGTTTTGTCGTTTTGTCGT-3′ (SEQ ID N^{o}: 1). In certain embodiments, a LLONC, where the oligonucleotide component has at least a 50% sequence identity to SEQ ID N^{o}: 1. In certain embodiments, LLONCs, where the oligonucleotide component has at least a: 60%, 70%, 75%, 80%, 85%, 90%, 91%, 92%, 93%, 94%, 95%, 96%, 97%, 98%, 99%, or at least a 99.9% sequence identity to SEQ ID N^{o}: 1. In all cases, the sequence should conserve at least one unmethylated CpG pair. The term “percent sequence identity” refers to the degree of identity between any given query sequence and a subject sequence.

One method comprises LLONCs, were the ON selected for conjugation has the sequence 5′-TCATCATTTTGTCATTTTGTCATT-3′ (SEQ ID N^{o}: 2). In certain embodiments, a LLONC, where the oligonucleotide component has at least a 50% sequence identity to SEQ ID N^{o}: 2. In certain embodiments, LLONCs, where the oligonucleotide component has at least a: 60%, 70%, 75%, 80%, 85%, 90%, 91%, 92%, 93%, 94%, 95%, 96%, 97%, 98%, 99%, or at least a 99.9% sequence identity to SEQ ID N^{o}: 2. In all cases, the sequence should conserve at least one active site with the sequence PyNTTTTGT. The term “percent sequence identity” refers to the degree of identity between any given query sequence and a subject sequence.

One method comprises LLONCs, were the ON selected for conjugation has the antisense sequence or has at least a: 90%, 91%, 92%, 93%, 94%, 95%, 96%, 97%, 98%, 99%, or at least a 99.9% sequence identity to the specific antisense ON.

In addition, the invention provides a treatment (in vivo or ex vivo) for regulating the immune response or suppressing-autoimmunity or suppressing acute or chronic inflammation or repairing a damaged organ or tissue, or cancer or ameliorating or suppressing pain in a mammal, especially a human being.

In certain embodiments, ex vivo treatments with LLONCs can be made on the appropriate cells (crude or purified) which can be autologous cells, comprising: autologous, allogeneic, haplotype matched, haplotype mismatched, haplo-identical, xenogeneic, cell lines or combinations thereof. In certain embodiments, the cells are B cells, T cells, antigen presenting cells, chimeric antigen receptor-T cells (CART), stem cells or combinations thereof.

In certain embodiments, the method further comprises administering one or more chemotherapeutic agents and/or the same or other active oligonucleotide with a LLONC. In certain embodiments, the method comprises administering to the subject one or more chemotherapeutic agents and/or radiotherapy and/or surgery.

During trauma, cancer, autoimmune disease and/or inflammatory disease, tissues and organs result damaged. Even after resolution of inflammation accumulated, damage and fibrosis may severally limit organ or tissue functionality. On the other hand, wounds or burns may be life threatening. In all these cases rapid tissue damage resolution, preserving functionality is highly desirable.

Thus, in one embodiment, the inventive LLONCs are used to treat an autoimmune disease, an inflammatory disease, or tissue damage in a mammal including a person. As used herein, the term “treatment,” refers to a procedure to obtain a desired pharmacologic effect. Preferably, the effect is therapeutic, that is, the effect partially or completely cures a disease.

Exemplary autoimmune diseases which may be treated by the LLONCs described at the present invention include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis, myocardial infarction, thrombosis, Wegener's granulomatosis, Takayasu's arteritis, Kawasaki syndrome, anti-factor VIII autoimmune disease, necrotizing small vessel vasculitis, microscopic polyangiitis, Churg and Strauss syndrome, pauci-immune focal necrotizing and crescentic glomerulonephritis, antiphospholipid syndrome, antibody-induced heart failure, thrombocytopeniaurpura, autoimmune hemolytic anemia, cardiac autoimmunity in Chagas' disease and anti-helper T lymphocyte autoimmunity.

Examples of autoimmune rheumatoid diseases include, but are not limited to rheumatoid arthritis and ankylosing spondylitis.

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome, diseases include, but are not limited to autoimmune diseases of the pancreas, Type 1 diabetes, autoimmune thyroid diseases, Graves' disease, spontaneous autoimmune thyroiditis, Hashimoto's thyroiditis, idiopathic myxedema, ovarian autoimmunity, autoimmune anti-sperm infertility⁷, autoimmune prostatitis and Type I autoimmune polyglandular syndrome.

Examples of autoimmune gastrointestinal diseases include, but are not limited to, chronic inflammatory intestinal diseases, celiac disease, colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis, primary biliary cirrhosis and autoimmune hepatitis.

Examples of autoimmune neurological diseases include, but are not limited to, multiple sclerosis, Alzheimer's disease, myasthenia gravis, neuropathies, motor neuropathies, Guillain-Barre syndrome and autoimmune neuropathies, myasthenia, Lambert-Eaton myasthenic syndrome, paraneoplastic neurological diseases, cerebellar atrophy, paraneoplastic cerebellar atrophy and stiff-man syndrome, non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies, immune neuropathies, acquired neuromyotonia, arthrogryposis multiplex congenita, neuritis, optic neuritis and neurodegenerative diseases.

Examples of autoimmune muscular diseases include, but are not limited to, myositis, autoimmune myositis and primary Sjogren's syndrome and smooth muscle autoimmune disease.

Examples of autoimmune nephric diseases include, but are not limited to, nephritis and autoimmune interstitial nephritis.

Examples of autoimmune diseases related to reproduction include, but are not limited to, repeated fetal loss.

Examples of autoimmune connective tissue diseases include, but are not limited to, ear diseases, autoimmune ear diseases and autoimmune diseases of the inner ear.

Examples of autoimmune systemic diseases include, but are not limited to, systemic lupus erythematosus and systemic sclerosis.

In certain embodiments, the compositions are administered to patients to prevent or treat an acute inflammatory disease, a chronic inflammatory disease, a neurodegenerative disease, a malignant tumor, or a benign tumor.

In certain embodiments of the present invention, the inflammatory disease comprises; psoriasis, rheumatoid arthritis (RA), Morbus Bechterew, multiple sclerosis (MS), systemic lupus erythematosus (SLE), Behcet's disease, uveitis, Sjogren syndrome, an inflammatory bowel disease (JBD), asthma, chronic obstructive pulmonary disease (COPD), neuropathic pain, atopic dermatitis, or allergy.

Examples of inflammatory diseases which may be treated by the present method include, but are not limited to, chronic inflammatory diseases and acute inflammatory diseases.

Inflammatory diseases associated with hypersensitivity: Examples of hypersensitivity include, but are not limited to, Type I hypersensitivity, Type II hypersensitivity, Type III hypersensitivity, Type IV hypersensitivity (Type I, II, and III reactions are the result of antibody actions, while type IV reactions involve T cell lymphocytes and cell-mediated immune responses), immediate hypersensitivity, antibody mediated hypersensitivity, immune complex mediated hypersensitivity, T lymphocyte mediated hypersensitivity and DTH.

Type I or immediate hypersensitivity, includes, for example, Allergic rhinitis (‘hay fever’); Allergic asthma; Allergic conjunctivitis; Anaphylaxis; Angioedema; Atopic dermatitis (eczema); Cephalosporin allergy; Eosinophilia; Food allergy; Penicillin allergy; Sweet itch; Urticaria (hives).

Type II hypersensitivity include, but are not limited to, Anti-factor VIII autoimmune disease, Antiphospholipid syndrome, Acquired neuromyotonia, Adrenoceptor antibodies in heart failure, Alzheimer's disease, Amyotrophic lateral sclerosis, Ankylosing spondylitis, Arteritis, Arthrogryposis multiplex congenital, Atherosclerosis, Autoimmune anti-sperm infertility, Autoimmune diseases of the gastrointestinal tract, Autoimmune diseases of the musculature, Autoimmune hemolytic anemia, Autoimmune hepatitis, Autoimmune myositis, Autoimmune polyendocrinopathies, Autoimmune reproductive diseases, Autoimmune thyroid diseases, Cardiovascular autoimmune diseases, Cardiovascular diseases, Celiac disease, Cerebellar atrophies, Cerebellar atrophy, Chronic inflammatory intestinal disease, Churg and Strauss syndrome, Crescentic glomerulonephritis, Diabetes, Dysimmune neuropathies, Encephalitis, Gastrointestinal diseases, Gilles de la Tourette syndrome, Glandular autoimmune diseases, Glandular diseases, Glomerulonephritis, Granulomatosis, Graves' disease, Guillain-Barre syndrome, Heart failure, Hemolytic anemia, Hepatic autoimmune diseases, Hepatic diseases, Idiopathic myxedema, Intestinal diseases, Kawasaki syndrome, Lambert-Eaton myasthenic syndrome, Microscopic, Motor neuropathies, Multiple sclerosis, Myasthenia gravis, Myasthenic diseases, Myocardial infarction, Myositis, Myxedema, Necrotizing small vessel vasculitis, Neurodegenerative diseases, Neurological autoimmune diseases, Neurological diseases, Neuromyotonia, Neuropathies and autoimmune neuropathies, Neuropathies, Non-paraneoplastic stiff man syndrome, Ovarian autoimmunity, Ovarian diseases, Pancreatic autoimmune diseases, Paraneoplastic cerebellar atrophy, Paraneoplastic neurological diseases, Pauci-immune focal necrotizing glomerulonephritis, Polyangiitis, Polyendocrinopathies, Primary biliary's cirrhosis, Progressive cerebellar atrophies, Rasmussen's encephalitis, Repeated fetal loss, Rheumatoid arthritis, Rheumatoid autoimmune diseases, Rheumatoid diseases, Sclerosis, Sjogren's syndrome, Smooth muscle autoimmune disease, Spondylitis, Spontaneous autoimmune thyroiditis, Sydeham chorea, Systemic autoimmune diseases, Systemic diseases, Systemic lupus erythematosus, Systemic sclerosis, Takayasu's arteritis, Thrombocytopenia purpura, Thrombosis, Thyroid diseases, Thyroiditis, Vasculitises, Wegener's granulomatosis;

Type III hypersensitivity include, but are not limited to, Arthus reaction, Farmer's Lung, Henoch-Schonlein purpura (IgA vasculitis), Polyarteritis nodosa, Post-streptococcal glomerulonephritis, Reactive arthritis, Rheumatoid Arthritis, Serum sickness and Systemic lupus erythematosus.

Type IV or T cell mediated hypersensitivity, include, but are not limited to, Anti-helper T lymphocyte autoimmunity, Autoimmune connective tissue diseases, Autoimmune ear disease, Autoimmune neurological diseases, Autoimmune, polyglandular syndrome, Autoimmune prostatitis, Autoimmune thrombocytopenia purpura, Autoimmune thyroid diseases, Biliary cirrhosis, Bullous pemphigoid, Bullous skin diseases, Cardiac autoimmunity in chagas' disease, Cardio vascular diseases, Chronic active hepatitis, Connective tissue diseases, Cutaneous diseases, Dermal diseases, Disease of the inner ear, Ear diseases, Glandular autoimmune diseases, Glandular diseases, Graves' disease, Hemolytic anemia, Hepatic autoimmune diseases, Hepatic diseases, Hepatitis, Interstitial nephritis, Multiple sclerosis, Myasthenia gravis, Nephric autoimmune diseases, Nephric diseases, Nephritis, Neuritis, Neurological diseases, Optic neuritis, Ovarian diseases, Pancreatic autoimmune diseases, Pancreatic diseases, Pemphigus foliaceus, Pemphigus vulgaris, Polyglandular syndrome, Primary biliary cirrhosis, Prostatitis, Rheumatoid arthritis, Rheumatoid diseases, Skin diseases, Stiff-man syndrome, Systemic autoimmune diseases, Systemic diseases, Systemic lupus erythematosus, Thyroid diseases, Type I autoimmune polyglandular syndrome and Type I diabetes,

Examples of delayed type hypersensitivity include, but are not limited to, contact dermatitis and drug eruption.

Examples of types of T lymphocyte mediating hypersensitivity⁷ include, but are not limited to, helper T lymphocytes and cytotoxic T lymphocytes.

Examples of helper T lymphocyte-mediated hypersensitivity include, but are not limited to, THT lymphocyte mediated hypersensitivity and TH2 lymphocyte mediated hypersensitivity.

In another embodiment, the inventive LLONCs are used as a prophylactic procedure to prevent an undesirable event, i.e., rejection of an organ, tissue or cells transplant. Regarding to this, the invention comprises administering a “prophylactically effective amount” of LLONCs to a person that has received a transplant in order to avoid rejection. “Prophylactically effective amount” refers to an amount effective, at dosages and for periods of time necessary, to attain a desired prophylactic result (e.g., prevention of graft vs. host disease).

In certain embodiments, a method of treating a subject comprising administering the LLONCs to the appropriate cells in an ex vivo treatment.

In certain embodiments, a method of treating a subject comprising administering the LLONCs oligonucleotide in combination with one or more therapeutic agents.

As used herein, the term “combination” embraces groups of compounds or non-drug therapies useful as part of a combination therapy. Such combination treatment is achieved by way of the simultaneous, sequential, or separate dosing of the individual components of the treatment. In certain examples, a composition of the invention is conjointly administered with one or more ant-inflammatory agents, chemotherapeutics, other therapeutics or combinations thereof.

In certain embodiments, a composition of the invention is administered in combination with a non-steroidal anti-inflammatory. Suitable non-steroidal anti-inflammatory compounds include, but are not limited to, piroxicam, diclofenac, etodoiac, indomethacin, ketoralac, oxaprozin, tolmetin, naproxen, flubiprofen, fenoprofen, ketoprofen, ibuprofen, mefenamic acid, sulindac, apazone, phenylbutazone, aspirin, celecoxib and rofecoxib.

According to the invention, in an ex vivo treatment, some appropriate cells previously activated with a LLONC, can be administered to a mammal, including a person, using standard cell transfer techniques. Examples of these techniques include autologous cell transplant, allogeneic cell transplant, and hematopoietic stem cell transplant. In a preferred embodiment, a composition comprising Breg-nov cells is transplant to a mammal via adoptive transfer methods (50). The LLONC activated cells can be administered to a human or other mammal in a suitable amount in order to achieve a desirable therapeutic effect.

The inventive LLONCs can be applied alone or in combination with other standard therapies. For example, they can be used in combination with immunosuppressive, anti-inflammatory or tissue repair agents for the treatment or prevention of a disease disclosed herein. Furthermore, LLONCs can be associated with devises aimed to repair injured organs and tissues including bones (e.g. patches for treatment of burns, patches for treatment of ulcers or dental implants).

The LLONCs may be applied to a surface of a device (e.g., a catheter) or contained within a pump, patch, or any other drug delivery device. The nucleic acids and vectors disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

### Combination Therapy

LLONC compositions of the invention may be combined in a pharmaceutical combination formulation, or dosing regimen as combination therapy, with a second compound, for example, chemotherapeutic agents, monoclonal antibodies, therapeutic vaccines, agents used in the treatment of autoimmune diseases, pain, inflammatory processes, etc. The second compound of the pharmaceutical combination formulation or dosing regimen preferably has complementary activities to the compounds of the invention such that they do not adversely affect the other(s). Such molecules are suitably present in combination in amounts that are effective for the purpose intended.

The combination therapy may be administered as a simultaneous or sequential regimen. When administered sequentially, the combination may be administered in two or more administrations. The combined administration includes co-administration, using separate formulations or a single pharmaceutical formulation, and consecutive administration in either order, wherein preferably there is a time period while both (or all) active agents simultaneously exert their biological activities. Suitable dosages for any of the above co-administered agents are those presently used and may be lowered due to the combined action (synergy) of the newly identified agent and other chemotherapeutic agents or treatments.

The combination therapy may provide “synergy” and prove “synergistic”, e.g. the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g. by different injections in separate syringes. In general, during alternation therapy, an effective dosage of each active ingredient is administered sequentially, e.g. serially, whereas in combination therapy, effective dosages of two or more active ingredients are administered together.

As an example, the agent may be administered in combination with surgery to remove an abnormal proliferative cell mass. As used herein, ‘in combination with surgery” means that the agent may be administered prior to, during or after the surgical procedure. Surgical methods for treating epithelial tumor conditions include intra-abdominal surgeries such as right or left hemicolectomy, sigmoid, subtotal or total colectomy and gastrectomy, radical or partial mastectomy, prostatectomy and hysterectomy. In these embodiments, the agent may be administered either by continuous infusion or in a single bolus. Administration during or immediately after surgery may include a lavage, soak, or perfusion of the tumor excision site with a pharmaceutical preparation of the agent in a pharmaceutically⁷ acceptable carrier. In some embodiments, the agent is administered at the time of surgery as well as following surgery in order to inhibit the formation and development of metastatic lesions. The administration of the agent may continue for several hours, several days, several weeks, or in some instances, several months following a surgical procedure to remove a tumor mass.

The subjects can also be administered the agent in combination with non-surgical anti-proliferative (e.g., anti-cancer) drug therapy. In one embodiment, the agent may be administered with a vaccine (e.g., anti-cancer vaccine) therapy. In one embodiment, the agent may be administered in combination with an anti-cancer compound such as a cytostatic compound. A cytostatic compound is a compound (e.g., a nucleic acid, a protein) that suppresses cell growth and/or proliferation. In some embodiments, the cytostatic compound is directed towards the malignant cells of a tumor. In yet other embodiments, the cytostatic compound is one that inhibits the growth and/or proliferation of vascular smooth muscle cells or fibroblasts. In some embodiments LLONCs are used for reducing the toxic secondary effects of citostatics.

Suitable anti-proliferative drugs or cytostatic compounds to be used in combination with the agents of the invention include anti-cancer drugs. Anti-cancer drugs are well known and include: Acivicin; Aclarubicin; Acodazole Hydrochloride; Acronine; Adozelesin; Aldesleukin; Altretamine; Ambomycin; Ametantrone Acetate; Aminoglutethimide; Amsacnne; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycm; Cisplatin; Cladribine; Cnsnatol Mesylate; Cyclophosphamide; Cytarabine; Dacarbazine; Dactinomycin; Daunorubicin Hydrochloride; Decitabine; Dexorrnaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabme; Gemcitabme Hydrochloride; Hydroxyurea; Idarabicin Hydrochloride; Ifosfamide; Ilmofosine; Interferon Alfa-2a; Interferon Alfa-2b; Interferon Aifa-nl; Interferon Alfa-n3; Interferon Beta-Ia; Interferon Gamma-Ib; Ipropiatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillm; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride: Pyrazofurin; Riboprme; Rogietimide; Safingol; Safingol Hydrochloride: Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Taxotere; Tecogalan Sodium; Tegafur; Teioxantrone Hydrochloride; Temoporfin; Teniposide; Teroxirone; Testolactone; Thiamiprine; Thioguanine; Thiotepa; Tiazofurin; Tirapazamine; Topotecan Hy drochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinflunine; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

The LLONCs of this invention are useful as adjuvants in a vaccine formulation comprising one or more antigens. In embodiments of this aspect, the vaccine formulation can be liquid or lyophilized in dosage form. Many dosage forms are known in the art and can be applied herein. In these preparations, the oligonucleotides of this invention may be combined with other immunostimulant compounds. Examples of well-known immunostimulants are: □-interferon, (3-interferon, y-interferon, granulocyte macrophage colony stimulator factor (GM-CSF), interleukin 2 (IL2), interleukin 12 (IL12) and CpG oligonucleotide, just as an example.

An aspect of this invention is the use of the present oligonucleotides for the manufacture of a medicament for vaccinating an animal. The animal can be vaccinated prophylactically or therapeutically. A prophylactic vaccine is designed to elicit protection from a disease caused by an infectious agent through the induction of specific immunity. A therapeutic vaccine is designed to induce remission of an illness (i. e. a tumor and metastasis or illness associated with an infectious agent like the human immunodeficiency virus).

In preferred embodiments, the antigenic component of the vaccine is one or more antigens, either natural or recombinant, of viruses like: Human immunodeficiency viruses, such as HIV-1 and HIV-2, coronavirus, polio viruses, hepatitis A virus, human coxsackie viruses, rhinoviruses, echoviruses, equine encephalitis viruses, rubella viruses, dengue viruses, encephalitis viruses, yellow fever viruses, coronaviruses, vesicular stomatitis viruses, rabies viruses, ebola viruses, parainfluenza viruses, mumps virus, measles virus, respiratory syncytial virus, influenza viruses, Hantaan viruses, bunga viruses, hemorrhagic fever viruses, reoviruses, orbiviuises, rotaviruses, Hepatitis B virus, parvoviruses, papilloma viruses, polyoma viruses, adenoviruses, herpes simplex virus (HSV) 1 and 2, varicella zoster virus, cytomegalovirus (CMV), variola viruses, vaccinia viruses, pox viruses, African swine fever virus, the unclassified agent of delta hepatitis, the agents of non-A, non-B hepatitis; or one or more antigens of infectious bacteria like: Helicobacter pylori, Borrelia burgdorferi, Legionella pneumophila, Mycobacterium tuberculosis, Mycobacterium bovis (BCG), Mycobacterium avium, Mycobacterium intracellulare, Staphylococcus aureus, Neisseria gonorrhoeae, Neisseria meningitidis, Listeria monocytogenes, Streptococcus pyogenes, Streptococcus pneumoniae, Haemophilus influenzae, Moraxella catharralis, Klebsiella pneumoniae, Bacillus anthracis, Corynebacterium diphtheriae, Clostridium perfringers, Clostridium tetani, Enterobacter aerogenes, Klebsiella pneumoniae, Pasteurella multocida, and Treponema pallidum; of infectious fungi like: Cryptococcus neoformans, Histoplasma capsulatum, Coccidioides immitis, Blastomyces dermatitidis, Candida albicans; of infectious protists like: Plasmodium falciparum, Trypanosoma cruzi, Leishmania donovani and Toxoplasma gondii. and human tumoral cells.

The method of vaccination includes administering one or more of the LLONCs of this invention and one or more antigens, that is, the vaccine can be designed against one disease target or a combination of disease targets.

Another aspect of this invention is a method of treatment of a person with vaccine for treating or prevent a tumor disease. The method of vaccination includes administering one or more of the LLONCs of this invention and one or more antigens characteristic of the tumor illness. Examples of tumor disease to be treated with these vaccines are: Chronic Myelogenous Leukemia, Precursor B-lymphoblastic lymphoma, B-cell chronic lymphocytic leukaemia, Lymphoplasmacytic lymphoma, Mantle cell lymphoma, Follicle center lymphoma, (follicular and diffuse), Marginal zone-B lymphoma, Extranodal lymphoma, Nodal marginal zone B-cell lymphoma, Splenic marginal zone B-cell lymphom, Hairy cell leukaemia, Plasmocytoma, Diffuse large B-cell lymphoma, Burkitt's lymphom, High grade B-cell lymphom, Burkitt like, Melanoma, Kaposis Sarcoma, Multiple Myeloma, Renal Cell Carcinoma, Bladder Cancer, Lung Cancer, Skin Cancer, Breast Cancer, Colon Cancer and Uterus Cancer.

In certain embodiments, the composition comprises LLONCs or any kind of cells which have been cultured ex vivo or a combination of both and one or more second, third, fourth, fifth etc., agents

With respect to treatment of autoimmune disease, excessive and prolonged activation of immune cells, such as T and B lymphocytes, and overexpression of the master proinflammatory cytokine tumor necrosis factor alpha (TNF), together with other mediators such as interlukin-6 0L-6), interlukin-1 (IL-1), and interferon gamma (IFN-γ), play a central role in the pathogenesis of autoimmune inflammatory responses in rheumatoid arthritis (RA), inflammatory bowel disease (IBD), Crohn's disease (CD), and ankylosing spondylitis (AS).

Non-steroidal anti-inflammatory drugs (NSAIDs), glucocorticoids, disease-modifying antirheumatic drugs (DMARDs) are traditionally used in the treatment of autoimmune inflammatory diseases. NSAIDs and glucocorticoids are effective in the alleviation of pain and inhibition of inflammation, while DMARDs have the capacity of reducing tissue and organ damage caused by inflammatory responses. More recently, treatment for RA and other autoimmune diseases has been revolutionized with the discovery that TNF is critically important in the development of the diseases. Anti-TNF biologies (such as infliximab, adalimumab, etanercept, golimumab, and certolizumab pepol) have markedly improved the outcome of the management of autoimmune inflammatory diseases. Other more powerful immunosuppressant drugs, such as methotrexate, cyclophosphamide, and azafhioprine can also be used in combination therapies.

According to the invention, LLONCs may be administered prior to, concurrent with, or following the other therapeutic compounds or therapies. The administration schedule may involve administering the different agents in an alternating fashion. In other embodiments, the agent may be delivered before and during, or during and after, or before and after treatment with other therapies. In some cases, the agent is administered more than 24 hours before the administration of the second agent treatment. In other embodiments, more than one anti-proliferative therapy or an autoimmune therapy may be administered to a subject. For example, the subject may receive the agents of the invention, in combination with both surgery and at least one other anti-proliferative compound. Alternatively, the agent may be administered in combination with more than one anti-cancer drug.

### Pharmaceutical Compositions

In certain embodiments, the present invention provides for a pharmaceutical composition comprising a LLONC as identified herein. The composition can be suitably formulated and introduced into a subject or the environment of a cell (e.g., immune cell, lymphs, a neoplasia, a cancer cell or a tumor) by any means recognized for such delivery.

Such compositions typically include the agent and a pharmaceutically acceptable carrier. As used herein the language “pharmaceutically acceptable carrier” includes saline, solvents, dispersion media, coatings, antibacterial and antifungal agents, isotonic and absorption delaying agents, liposomes, organic or mineral micro or nanoparticles and the like, compatible with pharmaceutical administration. Supplementary active compounds can also be incorporated into the compositions.

A pharmaceutical composition is formulated to be compatible with its intended route of administration (see below). Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Compositions of the present invention are administered to subjects in a variety of routes including but not limited to: oral administration, intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, or subcutaneous administration. The composition may be administered directly into the cancerous tumor, or in some embodiments can be administered to the immune cell.

Sterile injectable solutions can be prepared by incorporating the active compound in the required amount in a selected solvent with one or a combination of ingredients enumerated above (or equivalents), as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as pail of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

As defined herein, a therapeutically effective amount of LLONCs of the invention targeting a disease or disorder (i.e., an effective dosage) depends on the target disease or disorder selected. For instance, single dose amounts of a composition of the invention targeting a disease or disorder in the range of approximately 1 μg to 1000 mg/Kg may be administered; in some embodiments, 10, 30, 100, or 1000 pg/Kg, or 10, 30, 100, or 1000 ng/Kg, or 10, 30, 100, or 1000 μg/Kg, or 10, 30, 100, or 500 mg/Kg may be administered.

A therapeutically effective amount of the LLONCs of the present invention can be determined by methods known in the art. The therapeutically effective quantities of a pharmaceutical composition of the LLONCs will depend on the age and on the general physiological condition of the patient and the route of administration. In certain embodiments, the therapeutic doses will generally be between about 1 pg/Kg and 500 mg/Kg and preferably between about 1 □g/Kg and 50 mg/Kg. Other ranges may be used, including, for example, 0.050-500 mg/Kg, 0.5-100 mg/Kg and 1-20 mg/Kg.

Administration may be a single dose, multiple doses spaced at intervals to allow for a biological response to occur, once a day, twice a day, or more often, and may be decreased during a maintenance phase of a disease or disorder, e.g. once every second or third day instead of every day or twice a day. The dose and the administration frequency will depend on the clinical signs, which confirm maintenance of the remission phase, with the reduction or absence of at least one or more preferably more than one clinical sign of the acute phase known. Certain factors may influence the dosage and timing required to effectively treat a patient, including but not limited to the severity of the disease or disorder, previous treatments, the general health and/or age of the subject, and other diseases present. Moreover, treatment of a patient with a therapeutically effective amount of the compositions embodied herein can include a single treatment or, optionally, can include a series of treatments.

The compositions of the invention could also be formulated as nanoparticle formulation. The compounds of the invention can be administered for immediate-release, delayed-release, modified-release, sustained-release, pulsed-release and/or controlled-release applications. The pharmaceutical compositions of the invention may contain from 0.01 to 99% weight-per volume of the active material. For administration by inhalation, the compounds are delivered in the form of an aerosol spray from pressured container or dispenser which contains a suitable propellant, e.g., a gas such as carbon dioxide, or a nebulizer (^{xxiv}).

Systemic administration can also be by transmucosal or transdermal means. For transmucosal or transdermal administration, penetrants appropriate for the barrier to be permeated are used in the formulation. Such penetrants are generally known substances, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active LLONCs are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. Liposomal suspensions (including liposomes targeted to infected cells with monoclonal antibodies to viral antigens) can also be used as pharmaceutically acceptable carriers. These can be prepared according to methods known to those skilled in the art (for example as described in lxxv).

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may van’ within this range depending upon the dosage form employed and the route of administration utilized. For a compound used in a method of the invention, the therapeutically effective dose can be estimated initially from cell culture assays. A dose may be formulated in animal models to achieve a circulating plasma concentration range that includes the ICsO (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

It can be appreciated that the method of introducing an agent into the environment of a cell will depend on the type of cell and the makeup of its environment. Suitable amounts of an agent must be introduced and these amounts can be empirically determined using standard methods.

The pharmaceutical compositions can be included in a kit, container, pack, or dispenser together with instructions for administration.

### Other Embodiments

From the foregoing description, it will be apparent that variations and modifications may be made to the invention described herein to adopt it to various usages and conditions. Such embodiments are also within the scope of the following claims.

### EXAMPLES

The following examples further illustrate the invention.

### Example 1: LLONC Synthesis and Properties

In order to analyze the effect of conjugation with different organic molecules for dianophore modification of Oligonucleotides, several molecules were synthetized. In

| | | Nomenclature | | | | | | |
|---|---|---|---|---|---|---|---|---|
| Molecule | Description | HA(50) | HA(100) | HA(1000) | PEG(20) | PEG(40) | Myr | Palm |
| ON 1 | 5′-TCATCATTTTGTCATTTTGTCATT~[Amino C3]-3′ | HA(50)-ON 1 | HA(100)-ON 1 | HA(1000)-ON 1 | PEG(20)-ON 1 | PEG(40)-ON 1 | Myr-ON 1 | Palm-ON 1 |
| | fully phosphodiester | | | | | | | |
| ON 2 | 5′-ACATCATTTTGTCATTTTGTCATT~[Amino C3]3′ | HA(50)-ON 2 | HA(100)-ON 2 | HA(1000)-ON 2 | PEG(20)-ON 2 | PEG(40)-ON 2 | Myr-ON 2 | Palm-ON 2 |
| | fully phosphodiester | | | | | | | |
| ON 3 | 5′-TTTTTTTTTTTTCATTTTGTGGGG~[Amino C3]3′ | HA(50)-ON 3 | HA(100)-ON 3 | HA(1000)-ON 3 | PEG(20)-ON 3 | PEG(40)-ON 3 | Myr-ON 3 | Palm-ON 3 |
| | fully phosphodiester | | | | | | | |
| ON 4 | 5′-ACATCATTTTGTCATTTTGTAATT~[Amino C3]3′ | HA(50)-ON 4 | HA(100)-ON 4 | HA(1000)-ON 4 | PEG(20)-ON 4 | PEG(40)-ON 4 | Myr-ON 4 | Palm-ON 4 |
| | fully phosphodiester | | | | | | | |
| ON 5 | 5′-TCGTCGTTTTGTCGTTTTGTCGTT~[Amino C3]3′ | HA(50)-ON 5 | HA(100)-ON 5 | HA(1000)-ON 5 | PEG(20)-ON 5 | PEG(40)-ON 5 | Myr-ON 5 | Palm-ON 5 |
| | fully phosphodiester | | | | | | | |
| ON 6 | 5′-TCATCATTTTGTCATTTTGTCATT~[Amino C3]-3′ | HA(50)-ON 6 | HA(100)-ON 6 | HA(1000)-ON 6 | PEG(20)-ON 6 | PEG(40)-ON 6 | Myr-ON 6 | Palm-ON 6 |
| | fully phosphorothioated | | | | | | | |

the following table is indicated the nomenclature assigned to each entity.

The methodology for their synthesis, as well as their properties, are described in the following examples.

### Example 2: HA(100)-ON1 Synthesis

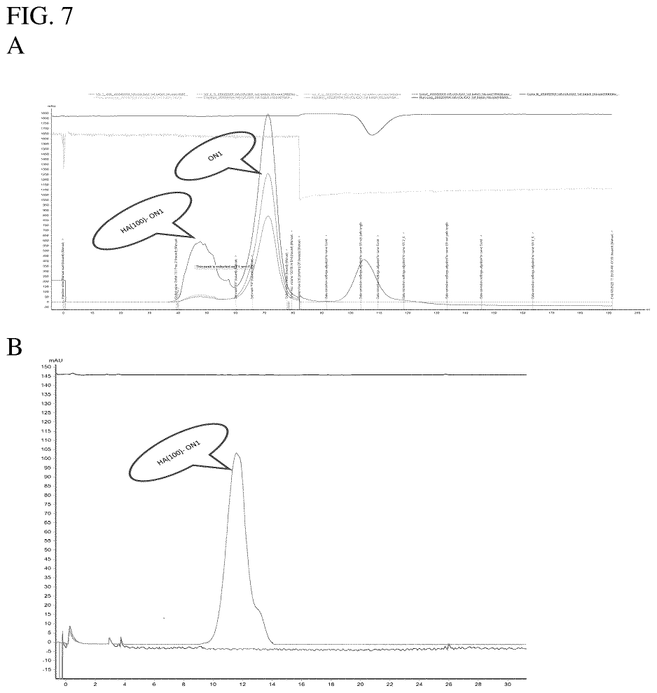

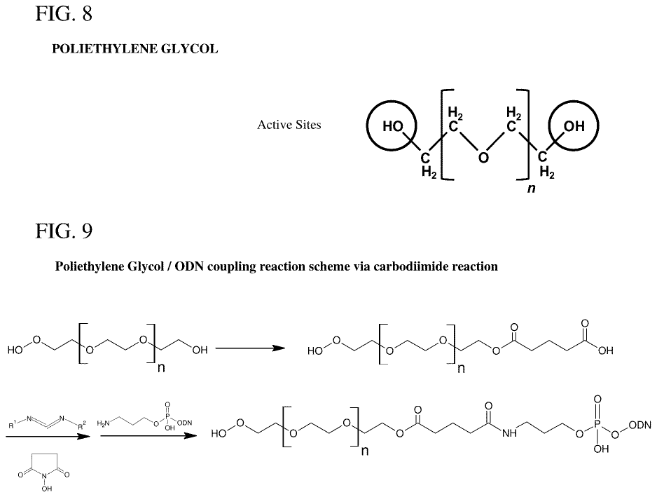

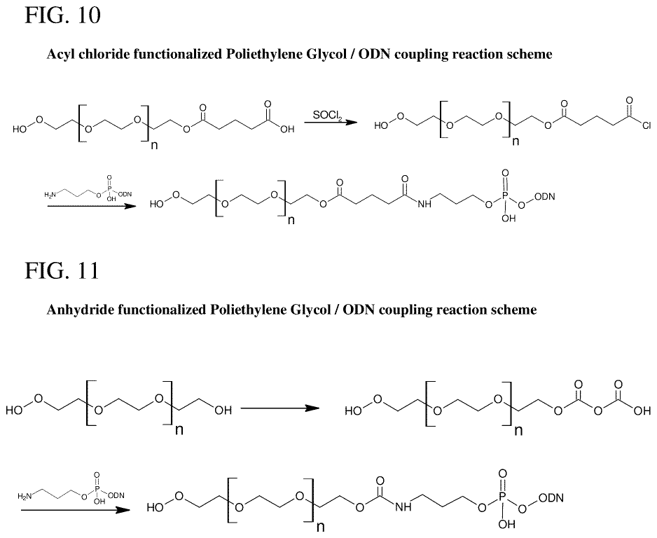

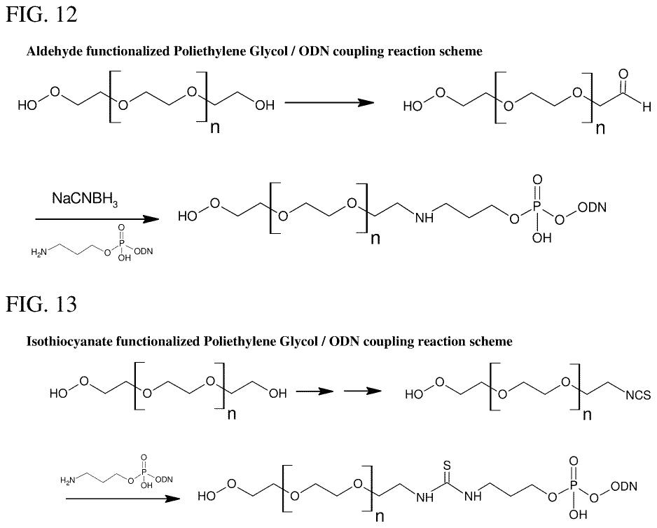

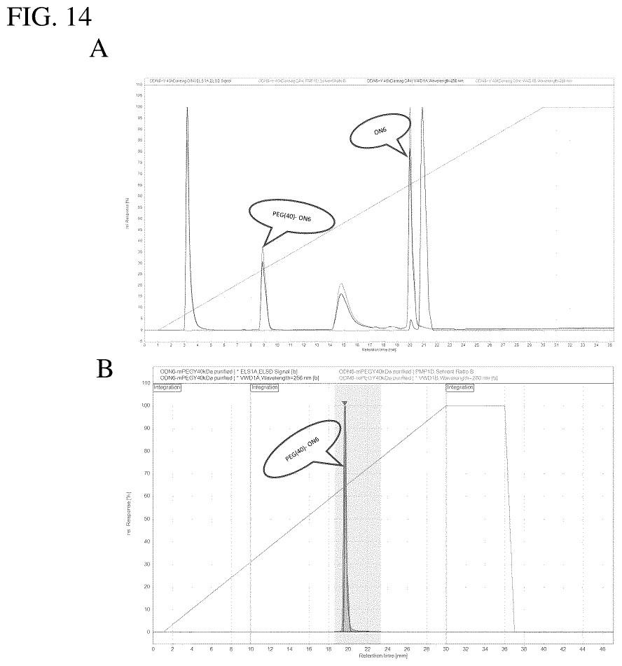

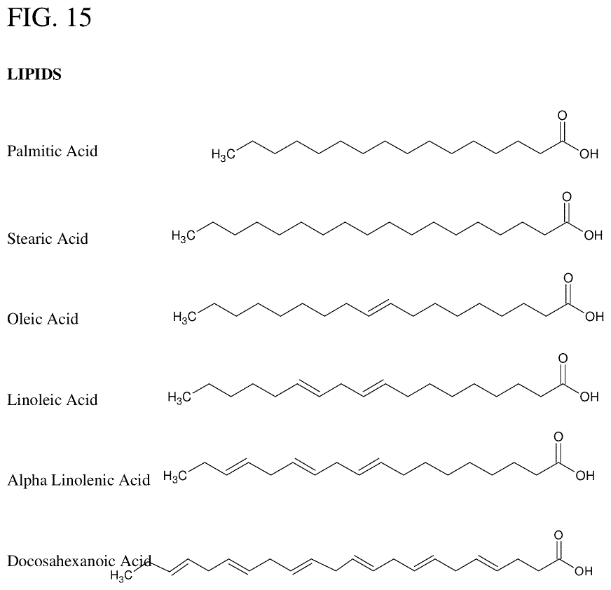

10 mg (25 μmol) of Hyaluronic Acid (MW 100 KDa) are dissolved in 5 ml of MES (morpholino ethanesulfonic acid) buffer at pH6 and at room temperature. 96 mg (2 mmol) of EDC-HCl (N-(3-Dimethylaminopropyl)-N′-ethylcarbodiimide hydrochloride), 108 mg (2 mmol) of sulfo NHS (N-hydroxysulfosuccinimide) and 5 mg (0.75 μmol) of ON 1 fosfodiester C3-amino modified at 3′ end are then added. The reaction mixture is stirred overnight (16 hours) at room temperature. In order to purify the product, the mixture is chromatographed in a size exclusion column (E.g. Hiload 16/60 Superdex 75 run is showed in FIG. 7.A.). The fractions of interest are pooled, the quantity of ON in the final product is calculated by UV absorbance at 260 nm, checked by analytical Size Exclusion-HPLC (FIG. 7.B.), fractionated and lyophilized.

### Example 3: HA(100)-ON6 Synthesis

10 mg (25 μmol) of Hyaluronic Acid (MW 100 KDa) are dissolved in 5 ml of MES (morpholino ethanesulfonic acid) buffer at pH6 and at room temperature. 96 mg (2 mmol) of EDC-HCl (N-(3-Dimethylaminopropyl)-N′-ethylcarbodiimide hydrochloride), 108 mg (2 mmol) of sulfo NHS (N-hydroxysulfosuccinimide) and 5.7 mg (0.75 μmol) of ON1 fully fosforothioated C3-amino modified at 3′ end are then added. The reaction mixture is stirred overnight (16 hours) at room temperature. In order to purify the product, the mixture is chromatographed in a size exclusion column (E.g. Hiload 16/60 Superdex 75). The fractions of interest are pooled, the quantity of ON in the final product is calculated by UV absorbance at 260 nm, checked by analytical Size Exclusion-HPLC fractionated and lyophilized.

### Example 4: HA(1000)-ON1 Synthesis

10 mg (25 μmol) of Hyaluronic Acid (MW 1 MDa) are dissolved in 5 ml of MES (morpholino ethanesulfonic acid) buffer at pH6 and at room temperature. 96 mg (5 mmol) of EDC-HCl (N-(3-Dimethylaminopropyl)-N′-ethylcarbodiimide hydrochloride), 108 mg (5 mmol) of sulfo NHS (N-hydroxysulfosuccinimide) and 5 mg (0.75 μmol) of ON 1 fosfodiester C3-amino modified at 3′ end are then added. The reaction mixture is stirred overnight (16 hours) at room temperature. In order to purify the product, the mixture is chromatographed in a size exclusion column (E.g. Hiload 16/60 Superdex 75). The fractions of interest are pooled, the quantity of ON in the final product is calculated by UV absorbance at 260 nm, checked by analytical Size Exclusion-HPLC fractionated and lyophilized.

### Example 5: HA(1000)-ON6 Synthesis

10 mg (25 μmol of basic disaccharide unit) of Hyaluronic Acid (MW 1 MDa) are dissolved in 5 ml of MES (morpholino ethanesulfonic acid) buffer at pH6 and at room temperature. 96 mg (5 mmol) of EDC-HCl (N-(3-Dimethylaminopropyl)-N′-ethylcarbodiimide hydrochloride), 108 mg (5 mmol) of sulfo NHS (N-hydroxysulfosuccinimide) and 5.7 mg (0.75 μmol) of ON6 fully fosforothioated C3-amino modified at 3′ end are then added. The reaction mixture is stirred overnight (16 hours) at room temperature. In order to purify the product, the mixture is chromatographed in a size exclusion column (E.g. Hiload 16/60 Superdex 75). The fractions of interest are pooled, the quantity of ON in the final product is calculated by UV absorbance at 260 nm, checked by analytical Size Exclusion-HPLC fractionated and lyophilized.

### Example 6: HA(50)-ON1 Synthesis

4 mg (10 μmol) of Hyaluronic Acid (MW 30-50 KDa) are dissolved in 2.5 ml of MES (morpholino ethanesulfonic acid) buffer at pH6 and at room temperature. 38 mg (2 mmol) of EDC-HCl (N-(3-Dimethylaminopropyl)-N′-ethylcarbodiimide hydrochloride), 43 mg (2 mmol) of sulfo NHS (N-hydroxysulfosuccinimide) and 7 mg (1 μmol) of ON1 fosfodiester C3-amino modified at 3′ end are then added. The reaction mixture is stirred overnight (16 hours) at room temperature. In order to purify the product, the mixture is chromatographed in a size exclusion column (E.g. Hiload 16/60 Superdex 75). The fractions of interest are pooled, the quantity of ON in the final product is calculated by UV absorbance at 260 nm, checked by analytical Size Exclusion-HPLC fractionated and lyophilized.

### Example 7: PEG-ON Synthesis (MW 40 KDa)

37 mg of oligo ON6 (any other equivalent ON—with an amino-linker can be processed in the same way) were weighed and dissolved in 5.0 ml MeOH plus 1.00 ml MeCN, 50 uL DIPEA and 50 uL water.

The solution was added to 1.3 g of solid (mPEG20K) 2Lys-COOSu and vortexed well; the ODN6 vial was rinsed with additional 500 uL MeOH and 500 uL MeCN and both volumes added to the reaction, shaking gently. Reaction is followed by HPLC: 5 μL of a 1:6 dilution of the reaction in water was checked by RP-HPLC-UV-ELSD

The purification was performed using Ion Exchange chromatography (E.g. Hitrap Q HP 5 ml) using 20 mM Na2HPO4 as mobile phase A and 2M NaCl in A as mobile phase B (FIG. 14). The reaction volume was purified in several runs; a large peak eluted at flowthrough containing the (mPEG20K) 2Lys-COOSu reagent mixed with the succinimide produced in the reaction. After few CV of washing with mobile phase A, a gradient going from 0% to 100% B in 30 minutes at 5 ml/min was performed; the PEGylated ODN6 appeared with a first peak at about 45 mS, immediately followed by the unreacted ODN6. The purified fraction containing PEG(40)-ON6 was desalted/purified by RP-HPLC. Briefly, a column PLRP-S 5 μm 4.6×250 mm was equilibrated in 10 mM NH4OAc in water at 1 ml/min and the entire fraction containing PEG(40)-ON6 was loaded into the column. After 10 CV of washing a gradient going to 100% acetonitrile in 30 min was started and the eluted peak absorbing at 256 nm was collected. The quantity of ON in the final product is calculated by UV absorbance at 260 nm, checked by analytical RP-HPLC-UV-ELSD (FIG. 14. B), fractionated and lyophilized.

### Example 8: PEG-ON Synthesis (MW 20 KDa)

37 mg of oligo ON1 (any other equivalent ON—with an amino-linker can be processed in the same way) were weighed and dissolved in 5.0 ml MeOH plus 1.00 ml MeCN, 50 uL DIPEA and 50 uL water.

The solution was added to 0.65 g of solid (mPEG20K) OSu and vortexed well; the ON1 vial was rinsed with additional 500 uL MeOH and 500 uL MeCN and both volumes added to the reaction, shaking gently. Reaction is followed by HPLC: 5 μL of a 1:6 dilution of the reaction in water was checked by RP-HPLC-UV-ELSD

The purification was performed using Ion Exchange chromatography (E.g. Hitrap Q HP 5 ml) using 20 mM Na2HPO4 as mobile phase A and 2M NaCl in A as mobile phase B. The reaction volume was purified in several runs; a large peak eluted at flowthrough containing the (mPEG20K) OSu reagent mixed with the succinimide produced in the reaction. After few CV of washing with mobile phase A, a gradient going from 0% to 100% B in 30 minutes at 5 ml/min was performed; the PEGylated ODN6 appeared with a first peak at about 45 mS, immediately followed by the unreacted ODN6. The purified fraction containing PEG(20)-ON1 was desalted/purified by RP-HPLC. Briefly, a column PLRP-S 5 μm 4.6×250 mm was equilibrated in 10 mM NH4OAc in water at 1 ml/min and the entire fraction containing PEG(20)-ON1 was loaded using a manual Rheodyne valve. After 10 CV of washing a gradient going to 100% acetonitrile in 30 min was started and the eluted peak absorbing at 256 nm was collected. The quantity of ON in the final product is calculated by UV absorbance at 260 nm, checked by analytical RP-HPLC-UV-ELSD, fractionated and lyophilized.

### Example 9: Palmitoyl-ON Synthesis

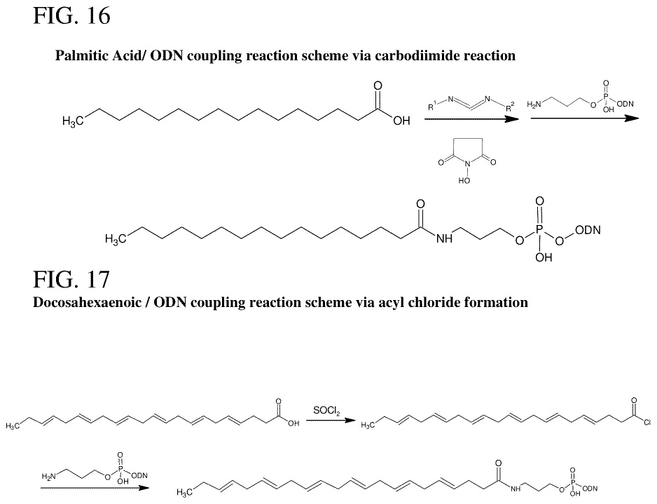

47 mg of lyophilized ON1 (any other equivalent ON—with an amino-linker can be processed in the same way) were introduced in a flask (DNAse RNase free). Then, 100 mg of Palm-OSu (Palmitic acid N-hydroxysuccinimide ester) crystallized were dissolved in 10 ml of methanol (100 mg, around 40× excess in molarity) and add the solution to the flask with the oligo. 200 ul of DNAse free water were added, gently vortexed and immediately added 150 ul of DIPEA (1% final) to bring it to basic pH. The mixture was incubated at RT (Scheme in FIG. 16). The reaction was followed by RPHPLC-MS on a Poroshell EC-C18 4.6×100 mm using 10 mM NH4OAc in water as mobile phase A and acetonitrile as Mobile phase B, with a gradient from 0 to 100% B in 20 min.

After 180 min the reaction was finished and the Palm-ON1 was purified in a semiprep Zorbax SBC18 9.4×250 mm, using 10 mM NH4OAc in water as mobile phase A and acetonitrile as Mobile phase B, with a gradient from 0 to 100% B in 20 min at 2 ml/min measuring OD at 256 and 280 nm. The reaction volume was purified in several runs; after injection of the reaction mix, the column was gently washed with Mobile phase A (10 mM NH4OAc pH7.8). After 1 CV two large peaks come out and both of them were collected. The last high peak is Palm-ON1 as evidenced immediately by mass spectrum of the collected fraction.

Then, fractions from several runs were pooled together, the quantity of ON in the final product is calculated by UV absorbance at 260 nm, checked by analytical RP-HPLC-UV-ELSD, fractionated and lyophilized.

### Example 10: Myristoil-ON Synthesis

47 mg of lyophilized ON1 (any other equivalent ON—with an amino-linker can be processed in the same way) were introduced in a flask (DNAse RNase free). Then, 100 mg of Myr-OSu (tetradecanoic acid N-hydroxysuccinimide ester) crystallized were dissolved in 10 ml of methanol (100 mg, around 40× excess in molarity) and add the solution to the flask with the oligo. 200 ul of DNAse free water were added, gently vortexed and immediately added 150 ul of DIPEA (1% final) to bring it to basic pH. The mixture was incubated at RT. The reaction was followed by RPHPLC-MS on a Poroshell EC-C18 4.6×100 mm using 10 mM NH4OAc in water as mobile phase A and acetonitrile as Mobile phase B, with a gradient from 0 to 100% B in 20 min.

Mass spectra of ON1 deconvolution gives 7401.03, while the more hydrophobic peak is 7610.85, perfectly in line with myristoylation (myristic acid=+228.37−water 18, totally +210)

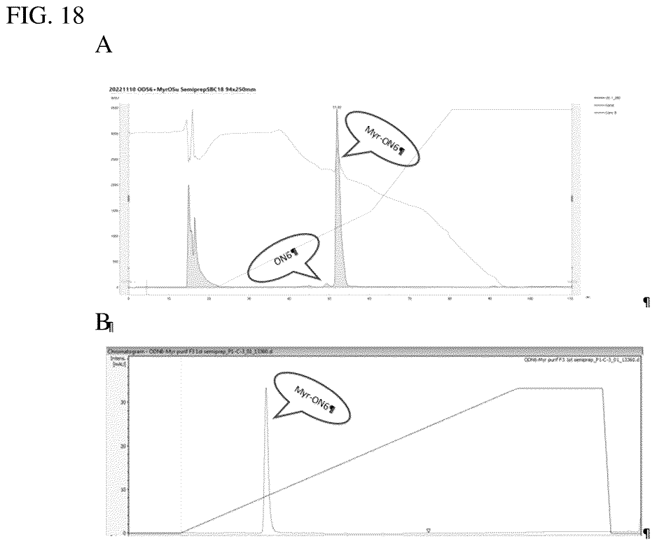

After 180 min the reaction was finished and the Myr-ON1 was purified in a semiprep Zorbax SBC18 9.4×250 mm, using 10 mM NH4OAc in water as mobile phase A and acetonitrile as Mobile phase B, with a gradient from 0 to 100% B in 20 min at 2 ml/min measuring OD at 256 and 280 nm (FIG. 18.A). The reaction volume was purified in several runs; after injection of the reaction mix, the column was gently washed with Mobile phase A (10 mM NH4OAc pH7.8). After 1 CV two large peaks come out and both of them were collected. The last high peak is Myr-ODN1 as evidenced immediately by mass spectrum of the collected fraction with MW=7610.46.

Then, fractions from several runs were pooled together, the quantity of ON in the final product is calculated by UV absorbance at 260 nm, checked by analytical RP-HPLC-UV-ELSD (FIG. 18. B), fractionated and lyophilized.

### Example 11: Docosahexaenoic-ON Synthesis

To a solution of docosahexaenoic acid (328.5 mg, 1 μmol) in acetonitrile (2 mL), 38 mg (2 mmol) of EDC-HCl and 43 mg of sulfo-NHS (2 mmol) were added (Scheme in FIG. 17). After 1 hour, 7 mg of ON fosfodiester C3-amino modified dissolved in 2 ml of MES buffer (pH6), and the mixture was stirred at room temperature for 5 hours. Then the Doc-ODN1 was purified in a semiprep Zorbax SBC18 9.4×250 mm, using 10 mM NH4OAc in water as mobile phase A and acetonitrile as Mobile phase B, with a gradient from 0 to 100% B in 20 min at 2 ml/min measuring OD at 256 and 280 nm. The reaction volume was purified in several runs; after injection of the reaction mix, the column was gently washed with Mobile phase A (10 mM NH4OAc pH7.8). After 1 CV two large peaks come out and both of them were collected. The last high peak is Doc-ODN1 as evidenced immediately by mass spectrum of the collected fraction.

Then, fractions from several runs were pooled together, the quantity of ON in the final product is calculated by UV absorbance at 260 nm, checked by analytical RP-HPLC-UV-ELSD, fractionated and lyophilized.

### Example 12: Hind Paw Inflammatory Pain Model

Adult Sprague-Dawley male rats (aprox. 250 g) were kept in a 12 h light-cycle, with water and food ad libitum.

A group of rats anesthetized with Isoflurane, the right hind paw was injected intra-dermally with 100 μl of Complete Freund Adjuvant (CFA, 1:1, dissolved in normal saline).

All rats received intra-plantar complete Freund's Adjuvant (CFA). 3 Days after, 4 animals have received a dose of 20 mg/kg IMT504 daily during 5 days. Other 4 animals have received only one dose of 0.4 mg/kg of the conjugate Palm-ON1 and 4 more, only one dose of 0.4 mg/kg of the conjugate HA(100) ON1. Then, they were tested periodically for mechanical and cold allodynia during 14 days.

CFA injured untreated (n=4) rats were used as controls. Untreated CFA rats received an intradermal hind paw injection of CFA and no further treatment.

Behavioral assessment was performed during daytime in all animals before any intervention (basal responses) and at different time-points after injury.

Nociceptive behavior assessment Thermal hyperalgesia and mechanical allodynia was evaluated in the inflammatory pain model. The basal measurements were obtained 2 days before the injury. Nociceptive evaluations were performed during daylight, when the animals were acclimated to the testing room and equipment for 30 minutes before the actual test.

Thermal sensitivity was evaluated by measuring paw withdrawal latencies to a radiant heat stimulus using the Hargreaves apparatus xxvi. The animals were placed in individual acrylic boxes over a tempered glass surface, and radiant heat source beneath the surface was aimed at the plantar surface of the hind paw. The seconds from initiation of thermal stimulus to removal of the hind paw (ipsilateral and contralateral to the site of injury) were recorded as paw withdrawal latency (PWL). A cutoff latency of 20 seconds was set to prevent tissue damage. Three trials on the same hind paw were performed with intervals of at least 6 minutes. The PWL was tested on days 1, 2, 4, 6, 8, 10, and 14 after CFA injection and all data was expressed as mean±S.E.M (all data underwent standard normality analysis).

Mechanical allodynia Von Frey filaments (MARSTOCK nerve test) were used to test mechanical allodynia in the hind paw (ipsilateral and contralateral to the site of injury)^{lxxvii}. The animals were placed in individual acrylic boxes with a mesh bottom, which permitted easy access to the plantar surface of the paws. Filaments were applied to the midplantar region of the hind paw, perpendicular to the plantar surface, maintaining constant force to cause a slight bend for 8 seconds. Abrupt paw withdrawal was recorded as a positive response. Based on the response pattern and the force of the final filament, the 50% response threshold was calculated using Dixon “up-down” method^{lxxviii} and was expressed as paw withdrawal threshold (PWT). The PWTs were tested on days 1, 2, 4, 6, 8, 10, and 14 after CFA injection and all data was expressed as mean±S.E.M (all data underwent standard normality analysis).

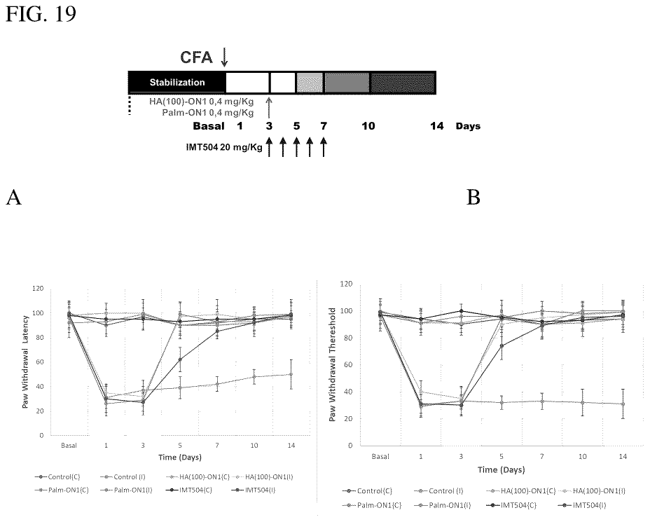

In CFA injured untreated animals, the ipsilateral PWL to thermal stimuli and the PWT to mechanical stimulation were significantly reduced in comparison with the contralateral paw and preinjured (basal) values (FIGS. 19.A and B). ODN treated animals significantly enhanced ipsilateral mechanical response threshold and thermal withdrawal latency after several days of treatment and further increased these parameters until they almost reached the levels detected at the contralateral paw. Additionally, in both cases, the behavior of prototypes Palm-ON1 and HA(100)-ON1 is identical, showing a total alleviation of pain in less than 24 h. The IMT504, however, has shown to reach to the same level of mitigation of pain in 3 days. Additionally, and surprisingly, the biological activity of those molecules is several hundred higher that the classical PyTTTGT ON (IMT504).

### Example 13: Effect of HA-ONs on the Brain Myelination

Experiments were conducted on a highly in-bred strain of Wistar rats handled in accordance with the NTH Guide for the Care and Use of Laboratory Animals. Twenty-one-day-old rats were housed in a temperature- and photoperiod-controlled room and fed milled chow without (control) or 0.6% (w/w) cuprizone (CPZ) for 14 days until 35 days of age. Four days before CPZ withdrawal, a group of animals were injected with saline solution (SS) and IMT504 (20 mg/kg) during five consecutive days. Also, four days before, CPZ withdrawal, a group of animals were injected with HA(1000)-ON1 (0.4 mg/kg) but only once instead the 5 day treatment with the comparative drug. Animals were sacrificed 7 days after CPZ withdrawal.

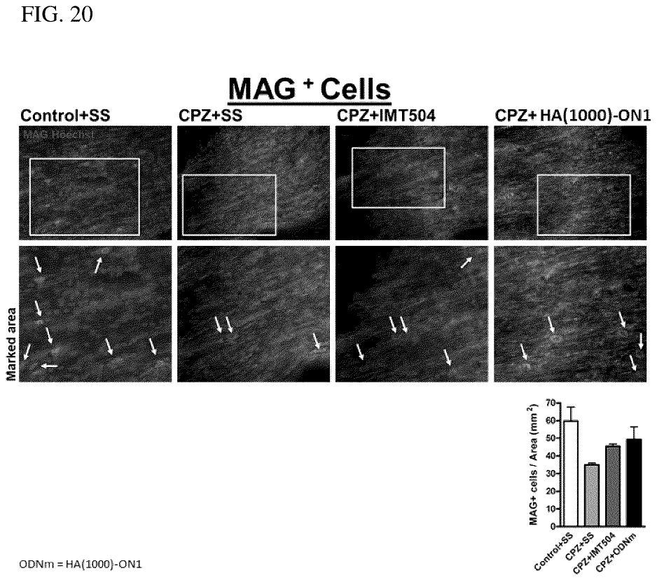

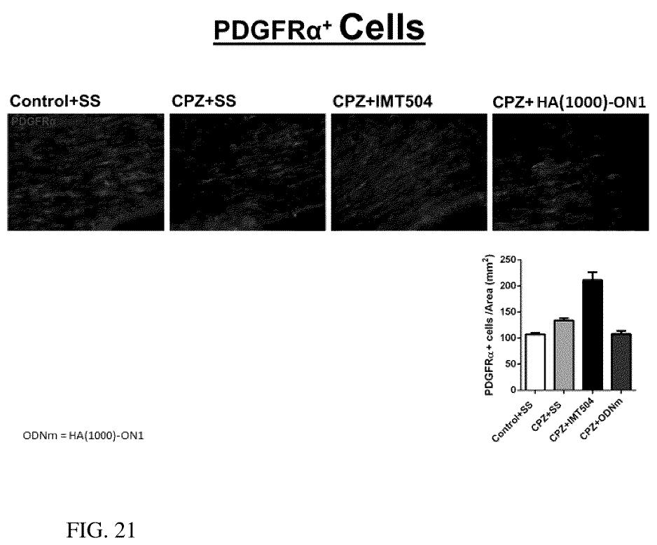

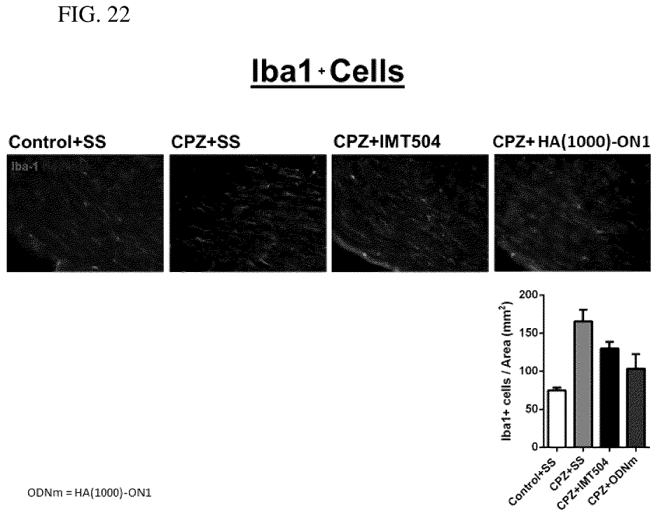

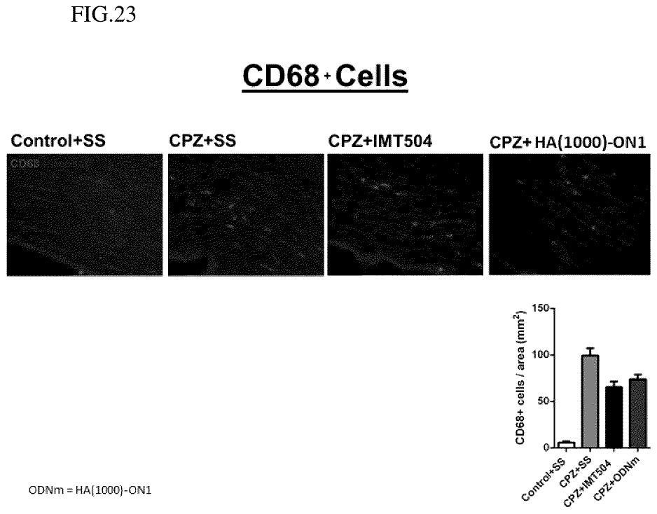

Briefly, 2 animals per experimental group were deeply anesthetized and perfused trans-intracardially with phosphate-buffered saline, pH 7.4 (PBS), followed by 4% (w/v) solution of paraformaldehyde in PBS. Brains were dissected out and post-fixed in the same solution overnight. After this, brains were thorough washed with PBS and cryoprotected by keeping them in 30% (w/v) sucrose in PBS. Brains were then frozen and used to obtain 30 um free-floating coronal sections using a Leica CM 1850 cryotome. Microscopic observations were conducted using an Olympus BX50 microscope and photographs were obtained with a CoolSnap digital camera. The Image Pro Plus software (version 5.5) was used for image analysis. FIGS. 20, 21, 22 and 23 describe the obtained results. These results indicated that both, IMT504 (100 mg/kg total trough 5 daily applications) or HA(1000)-ON1 (0.4 mg/kg total in only 1 shot) induce an increase in the population of mature oligodendrocytes involved in the remyelination of the CC of CPZ-demyelinated rats. Results also showed a decrease in the inflammatory response observed as a consequence of demyelination in the CC. This means that HA(1000)-ON1 appears to be, surprisingly, at least 250 times more biologically active. Moreover, the ON conjugated with the HA has a phosphodiester backbone while IMT504 has a phosphorothioate backbone (it must be recalled that the equivalent sequence of IMT504, but with a phosphodiester backbone, has only 30 times less activity).

### Example 14: Effect of Palm-ON1, PEG(20)-ON1, Palm-ON6 and PEG(20)-ON6 on the Brain Myelination

Experiments were conducted on a highly in-bred strain of Wistar rats handled in accordance with the NTH Guide for the Care and Use of Laboratory Animals. Twenty-one-day-old rats were housed in a temperature- and photoperiod-controlled room and fed milled chow without (control) or 0.6% (w/w) cuprizone (CPZ) for 14 days until 35 days of age. Four days before CPZ withdrawal, a group of animals were injected with saline solution (SS) and IMT504 (20 mg/kg) during five consecutive days. Also, four days before, CPZ withdrawal, 4 groups of animals were injected respectively with 0.4 mg/Kg of Palm-ON1, Palm-ON6, PEG(20)-ON1 and PEG(20)-ON6, but only once instead the 5 days treatment with the comparative drug. Animals were sacrificed 7 days after CPZ withdrawal.

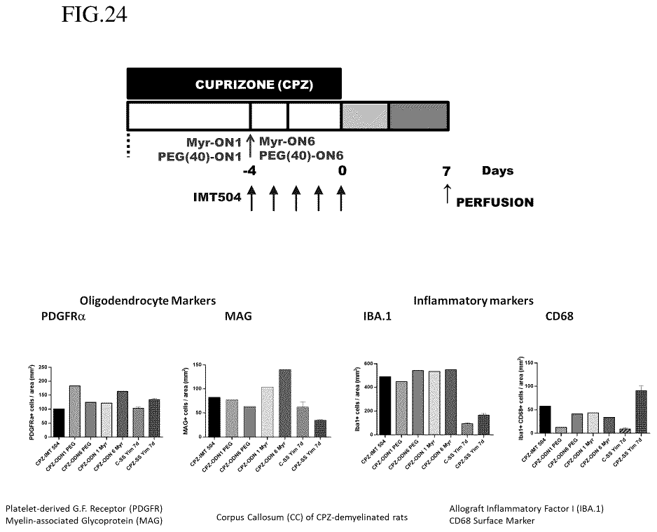

Brain treatments, procedures and analysis were conducted es described in the previous Example. FIG. 24 describe the obtained results. These indicates that all, IMT504 (100 mg/kg total trough 5 daily applications) or any tested LLONC (at 0.4 mg/kg total in only 1 shot) induce an increase in the population of mature oligodendrocytes involved in the remyelination of the CC of CPZ-demyelinated rats. Results also showed a decrease in the inflammatory response observed as a consequence of demyelination in the CC. This means that Palm-ON1, PEG(20)-ON1, Palm-ON6 and PEG(20)-ON6 appears to be, surprisingly, at least 250 times more biologically active. Moreover, the Palm-ON1 and PEG(20)-ON1 have a phosphodiester backbone while IMT504 has a phosphorothioate backbone (it must be recalled that the equivalent sequence of IMT504, but with a phosphodiester backbone, has only 30 times less activity).

### Example 15: PEG(20)-ON6 and Palm-ON6 Protection Against Cisplatin-Induced Toxicity in Mice

Balb/c mice (20-22 g, male) were maintained under pathogen free conditions with a 12 h light/dark cycle at 23° C. and they had free access to food and water. All animal procedures were performed in accordance with the Guidelines for Care and Use of Laboratory Animals of the National Institutes of Health.

Mice were randomly divided into seven groups (n=5; N=35), namely, Control CPt(−) group which was injected (sc) with 200 □l of 0.05 mM phosphate buffered saline (PBS) pH 7.4; Control CPt(+) group which was injected (sc) with 200 □l of 0.05 mM phosphate buffered saline (PBS) pH 7.4. IMT504 group which was injected (sc) with 200 □l of IMT504 (20 mg/ml) in PBS (total dose of 20 mg/kg animal weight) every day during the first 5 consecutive days. PEG(20)-ON1 200 group which was injected (sc) with 200 □l of PEG(20)-ON1 in PBS (200 g/kg animal weight) in only one shot. PEG(20)-ON1 20 group which was injected (sc) with 200 □l of PEG(20)-ON1 in PBS (20 g/kg animal weight) in only one shot. Palm-ON1 200 group which was injected (sc) with 200 □l of Palm-ON1 in PBS (200 g/kg animal weight) also in only one shot and Palm-ON1 20 group which was injected (sc) with 200 □l of Palm-ON1 in PBS (20 g/kg animal weight) also in only one shot.

Two days after, all animals, except those belonging to the group Control CPt (−) have received an intraperitoneal injection of cisplatin (20 mg kg-1). After the treatment with cisplatin each dead animal was autopsied immediately and the survived ones were sacrificed, and autopsied 14 days after Cisplatin treatment. Spleen and kidneys of any animal were collected and were kept in 4% paraformaldehyde for 24 h and then embedded in paraffin wax. Tissues were sectioned and stained with Masson's trichrome and hematoxylin and eosin (H&E) stains before examination under a light microscope.

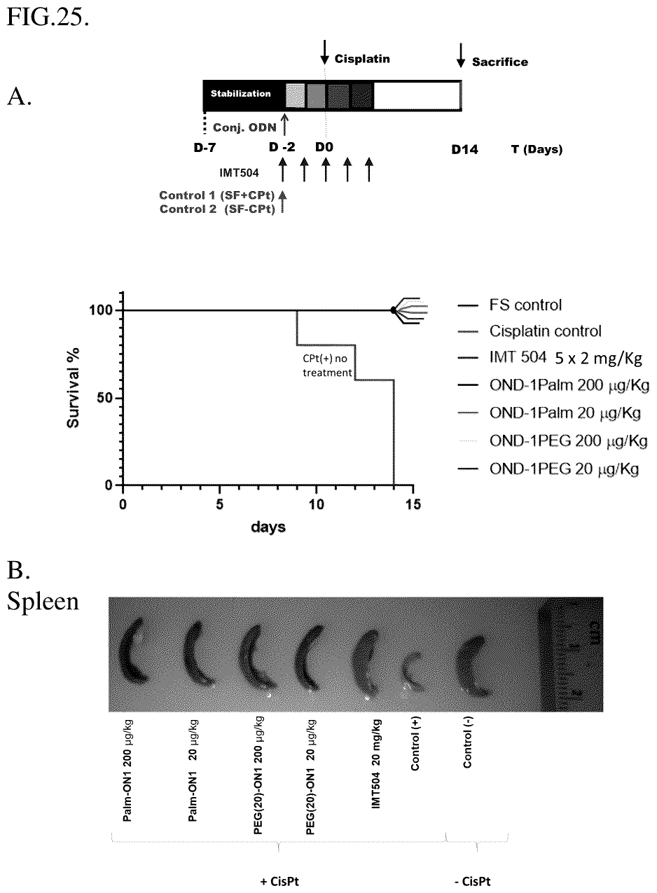

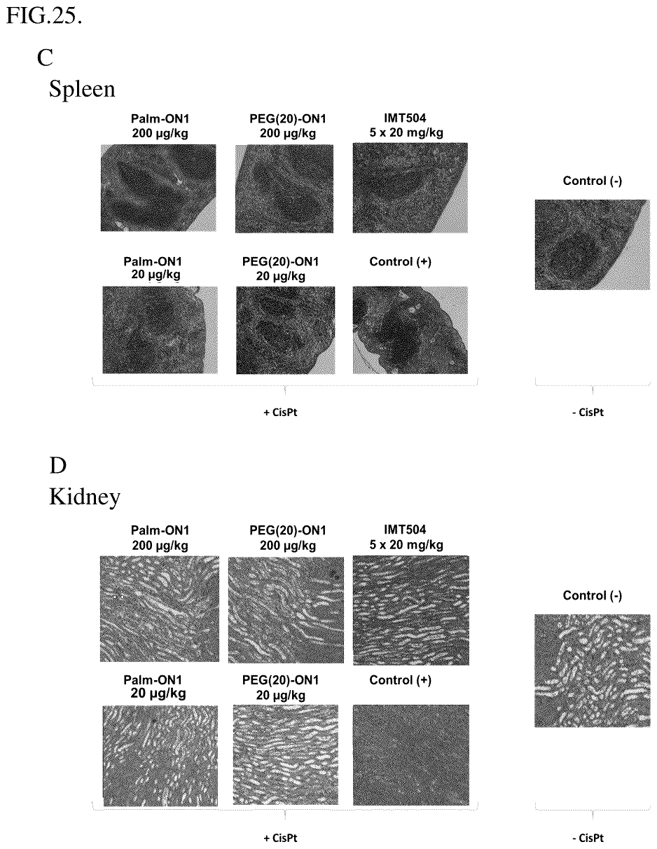

Survival curve of animals were drawn (FIG. 25A) and data were analyzed by a Kaplan-Meier test. The macroscopic (FIG. 25.B) and histological (FIG. 25.C) comparison of spleen from animals with all treatments are shown. Also, histological comparison of liver from animals with all treatments are shown in FIG. 25.D

Animals from group treated with five consecutive injections of 2 mg/kg (10 mg/Kg total=minimum protective dose) of mouse were protected from the damage caused by cisplatin (CPt). While the mice inoculated with CPt (CPt(+)) which do not receive any other treatment showed a behavior compatible with severe intoxication (finally evidenced by the death of all the animals in the group), the animals belonging to the IMT504 treated group showed a behavior similar to the control group (CPt(−)) and survived during the study period. Additionally, the histopathology of the organs from the sacrificed LLONC treated animals did not show significant differences with the CPt(−) control. However, the CPt(+) control without other treatment showed severe histological and morphological damage in the studied organs. In the same way, as in CPt(+) animals treated with IMT504, those treated with only one shot of PEG(20)-ON1 (200 g/kg animal weight), PEG(20)-ON1 (20 g/kg animal weight), Palm-ON1 (200 g/kg animal weight) and Palm-ON1 (20 g/kg animal weight) also did not show differences with the control CPt(−) group, neither in behavior, nor in mortality, nor in the morphological or histological analysis of the studied organs. Cisplatin is one of the most widely prescribed chemotherapeutic drugs, used to treat a wide range of pediatric and adult malignances and it is prescribed in nearly 50% of all tumor chemotherapies [^{lxxix}]. However, it has limited use in clinical practice due to various deleterious side effects. Currently, around 40 side effects of cisplatin have been reported, but the kidney is the most affected organ (in more than 60% of the treated patients). Then, the results shown high protection of kidney from the deleterious effects due to the cytostatic using LLONCs as therapeutic adjuvants. On the other hand, because the activity of classic immunomodulators, as well as that of LLONCs, depends on their action on B lymphocytes and the integrity of the spleen, the protection that these molecules provide on this organ would enhance their effectiveness. Additionally, it can be concluded that the LLONCs studied in the present trial were shown to be about 500 times more powerful than IMT504 in reducing or avoiding the toxic effects of Cisplatin.

### Example 16: Effect of HA-ONs as Adjuvant

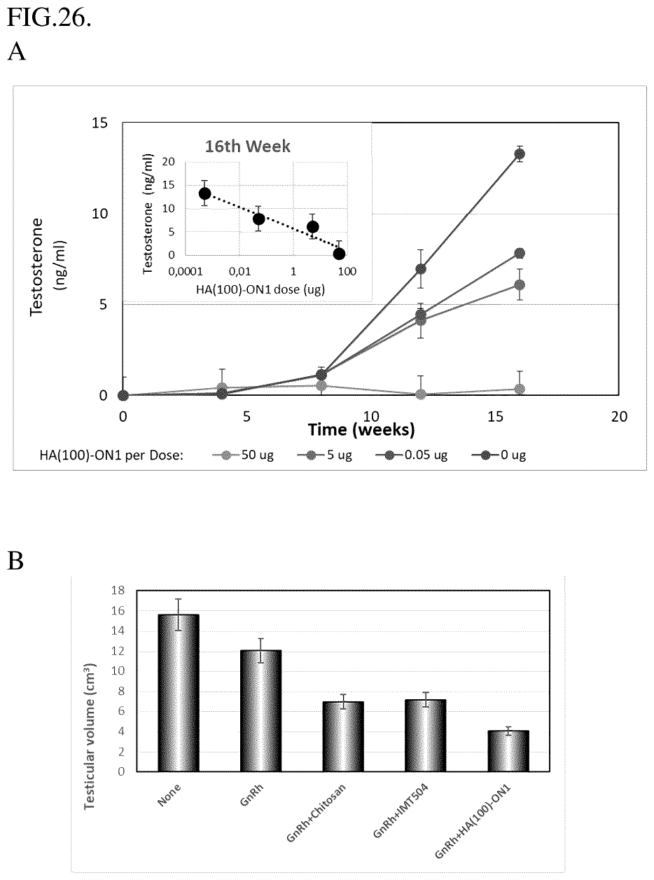

Young male Wistar rats, 4-week-old, were taken for immunization and assigned to various groups randomly on equitable basis of body weights. Recombinant gonadotropin-releasing hormone (GnRH) was combined with none (0.05 mM phosphate buffered saline (PBS)) or different adjuvant molecules, yielding different immunocontraceptive vaccines (300 □g GnRH alone, 300 □g GnRH+50 □g of chitosan, 300 □g GnRH+50 □g of IMT504 and 300 □g GnRH+50 □g of HA(100)-ON1). Each group of rats was injected subcutaneously (s.c.) with the corresponding composition and then, the same composition booster injections were given 4 weeks after the first shot. Sera collection following the first injection on Day 0, blood was collected by retro-orbital puncture after every 15 days and sera separated and stored at −20° C. until assayed for testosterone levels. Necropsy was performed 8 weeks after the first injection, testicles were extracted and measured with a caliper in all 3 dimensions (a, b and c) and the volume was calculated according to the formula V=(4/3)×π×(L/2)×(W/2)×(D/2), where L is the tumor length, W is the tumor width and D is the estimated tumor depth. Results in FIG. 26A. shows that also the group of animals vaccinated using GnRH plus HA(1000)-ON1 as adjuvant, had the lower levels of serum Testosterone and the insert in the figure allow to see also a dose/response behavior. On the other hand, FIG. 26 B shows that animals vaccinated using LLONC HA(1000)-ON1 as adjuvant shown much better inhibition of testicular growth. Both results have demonstrated that HA(1000)-ON1 adjuvant is much more potent than all other used here for developing an immunological reaction against GnRh.

### Example 17: Effect of Palm-ON1 and PEG(20)-ON1 as Adjuvants

Young male Wistar rats, 4-week-old, were taken for immunization and assigned to various groups randomly on equitable basis of body weights. Vero cell RABV vaccine (VeroRab™, Sanofi-Pasteur) containing ≥2.5 IU per dose was used for immunization of rats.

Each rat received a single subcutaneous injection containing 50 μl of a 1/125 dilution of the original vaccine. Control groups received the vaccine alone (control) and experimental groups received vaccine with the addition of 50 μg (IMT504 50) or 10 μg (IMT504 10) of ODN IMT504. Or 5 μg (ODN1-Palm 5) or 1 μg (ODN1-Palm 1) or 0.2 μg (ODN1-Palm 0.2) of Palm-ON1. Or 5 μg (ODN1-PEG 5) or 1 μg (ODN1-PEG 1) or 0.2 μg (ODN1-PEG 0.2) of PEG(20)-ON1

Each group of rats was injected subcutaneously (s.c.) with the corresponding composition and then, the same composition booster injections were given 2 and 4 weeks after the first shot.

At times 0, 14, 28 and 42 days, the rats were bled (collected by retro-orbital puncture) and sera separated and stored at −20° C. until assayed for specific total IgG antibody determination by ELISA.

Briefly, ELISA plates (Maxisorp™, Nunc) were coated overnight with 40 μg/ml of the recombinant rabies antigen. Residual protein-binding sites were blocked with carbonate-bicarbonate buffer containing 8% non-fat milk. Serum samples were diluted 1/200 with 0.05% Tween 20 in phosphate buffered saline (PBS) and plates incubated 2 h at 37° C. Total IgG was evaluated using Sigma A 5795 Ab conjugated with horseradish peroxidase. O-phenylendiamine dichloride solution (1 mg/ml) was finally added to the wells. Optical Density (OD) was measured at 490 nm.

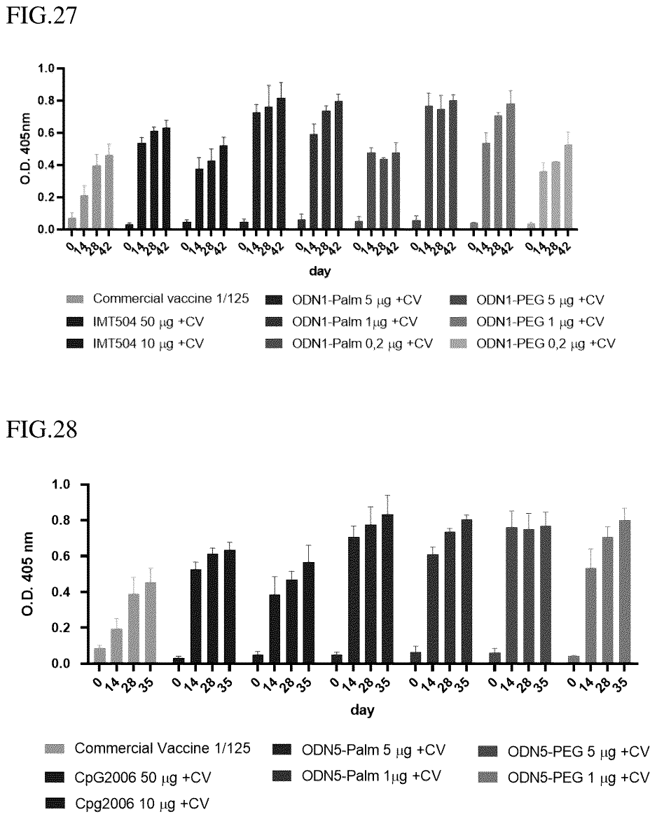

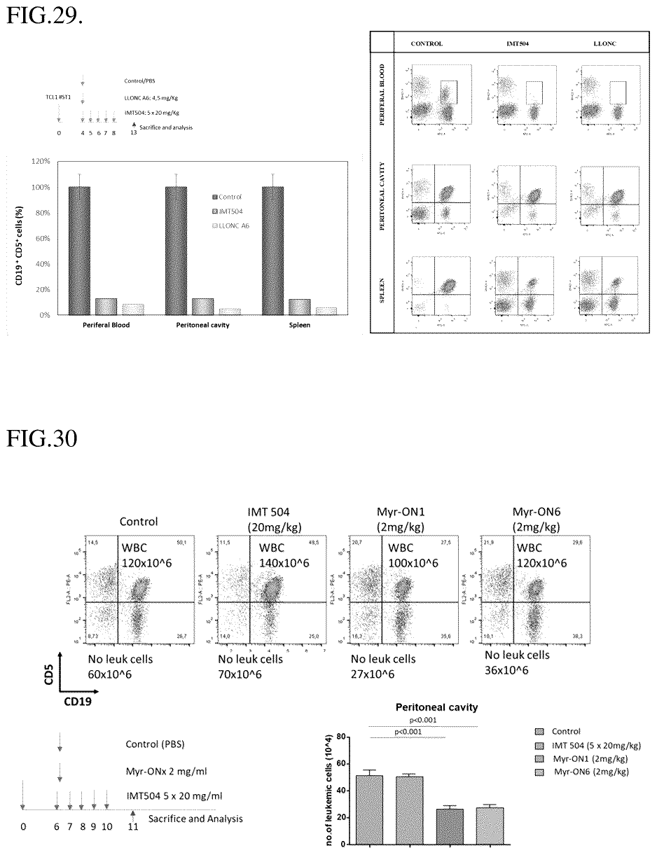

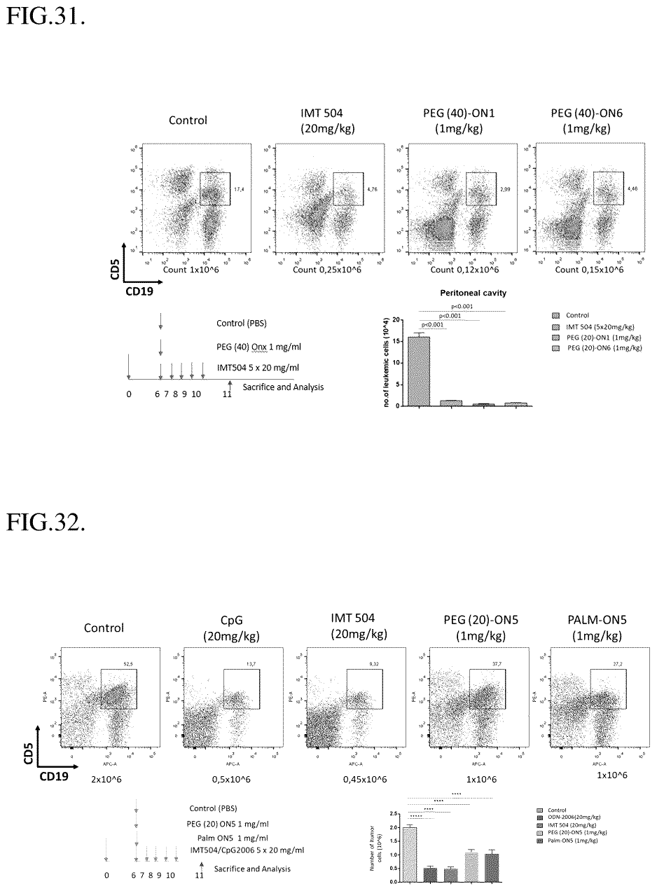

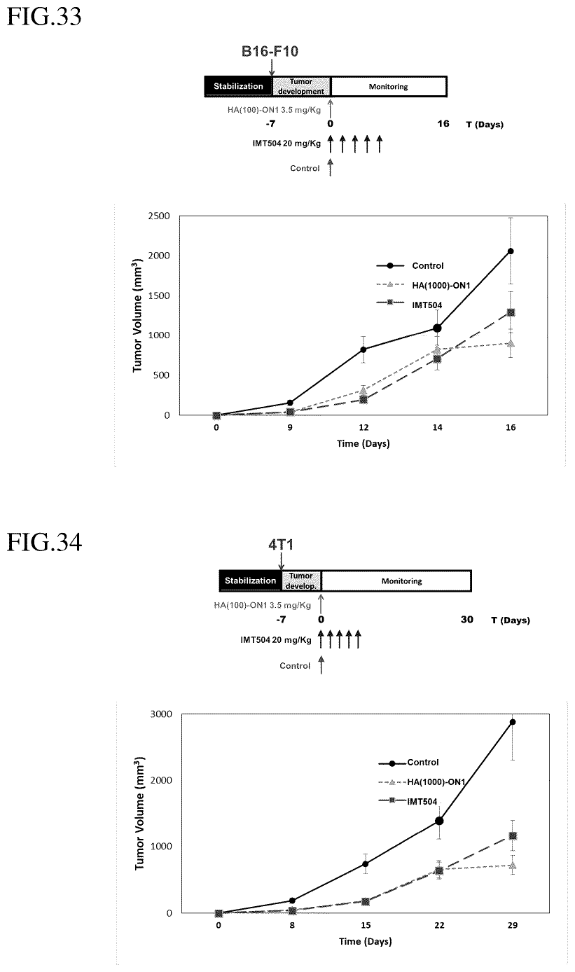

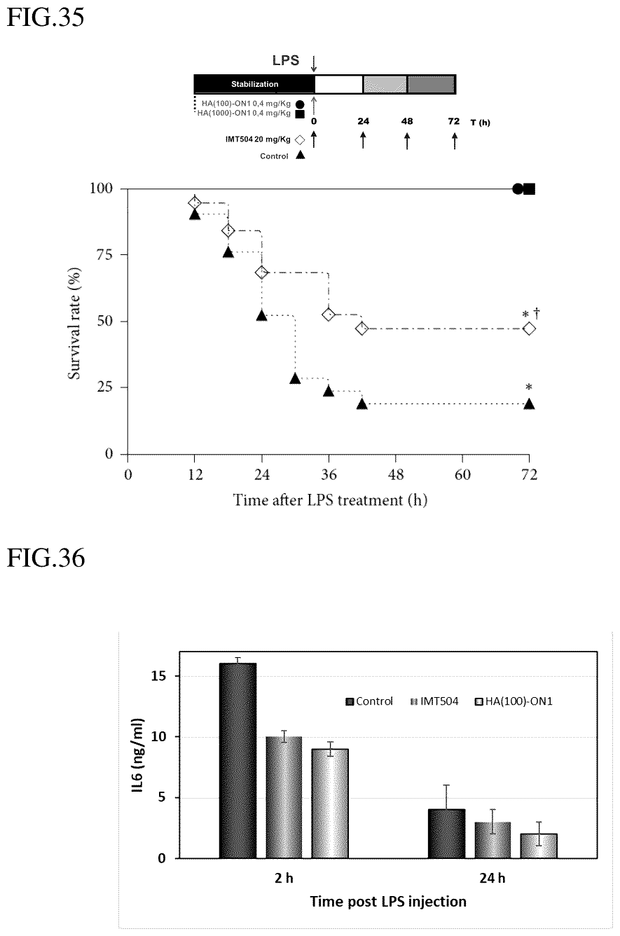

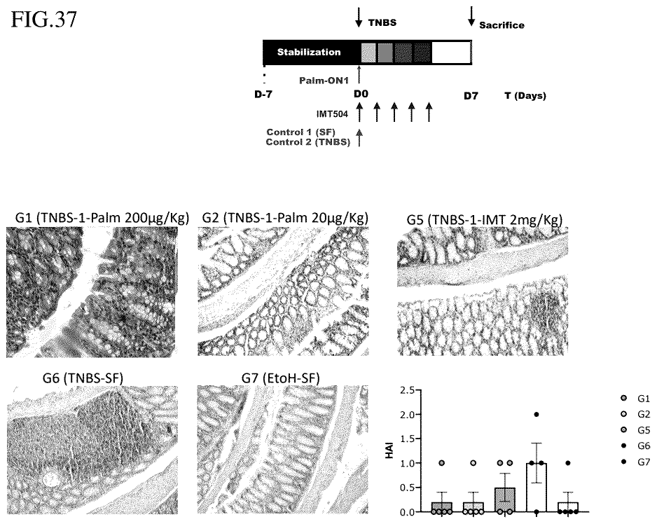

Results in FIG. 27. show that also the group of animals vaccinated using Rabic antigen plus LLONCs based on ODN1 (Palm or PEG(20)) as adjuvant, had the maximum levels of rabies specific total IgG antibody in serum. It can see also a dose/response behavior in every group. These results have demonstrated that LLONCs based on ODN1 (Palm or PEG(20)) adjuvated vaccines are much more potent (at least 50 times) than both original vaccine alone or IMT504 adjuvated. All adjuvated vaccines with ODN1 based complexes shows, additionally, the same behavior than those adjuvated with IMT504, which is to reach almost to the max. titles sooner than the control (commercial) vaccine. This phenomenon is really important because the rabies vaccine is mostly used as therapeutic vaccine.

### Example 18: Effect of Palm-CpG2006 and PEG(20)-CpG2006 as Adjuvants

Young male Wistar rats, 4-week-old, were taken for immunization and assigned to various groups randomly on equitable basis of body weights. Vero cell RABV vaccine (VeroRab™, Sanofi-Pasteur) containing ≥2.5 IU per dose was used for immunization of rats.

Each rat received a single subcutaneous injection containing 50 μl of a 1/125 dilution of the original vaccine. Control groups received the vaccine alone (control) and experimental groups received vaccine with the addition of 50 μg (CpG2006 50) or 10 μg (CpG2006 10) of ODN CpG2006. Or 5 μg (ODN5-Palm 5) or 1 μg (ODN5-Palm 1) or 0.2 ug (ODN5-Palm 0.2) of Palm-ON5. Or 5 μg (ODN5-PEG 5) or 1 μg (ODN5-PEG 1) or 0.2 μg (ODN5-PEG 0.2) of PEG(20)-ON5

Each group of rats was injected subcutaneously (s.c.) with the corresponding composition and then, the same composition booster injections were given 2 and 4 weeks after the first shot.

At times 0, 14, 28 and 42 days, the rats were bled (collected by retro-orbital puncture) and sera separated and stored at −20° C. until assayed for specific total IgG antibody determination by ELISA.

Briefly, ELISA plates (Maxisorp™, Nunc) were coated overnight with 40 μg/ml of the recombinant rabies antigen. Residual protein-binding sites were blocked with carbonate-bicarbonate buffer containing 8% non-fat milk. Serum samples were diluted 1/200 with 0.05% Tween 20 in phosphate buffered saline (PBS) and plates incubated 2 h at 37° C. Total IgG was evaluated using Sigma A 5795 Ab conjugated with horseradish peroxidase. O-phenylendiamine dichloride solution (1 mg/ml) was finally added to the wells. Optical Density (OD) was measured at 490 nm.

Results in FIG. 28 shows that also the group of animals vaccinated using Rabic antigen plus LLONCs based on ODN5 (Palm or PEG(20)) as adjuvant, had the maxim levels of rabies specific total IgG antibody in serum. It can see also a dose/response behavior in every group. These results have demonstrated that LLONCs based on ODN5 (Palm or PEG(20)) adjuvated vaccines are much more potent (at least 50 times) than both original vaccine alone or CpG2006 adjuvated. All adjuvated vaccines with CpG ODN5 based complexes shown, additionally, the same behavior than those adjuvated with CpG2006, which is to reach almost to the max. titles sooner than the control vaccine. This phenomenon is really important because the rabies vaccine is mostly used as therapeutic vaccine.

### Example 19: Effect of HA-ONs in Chronic Lymphocytic Leukemia

WT mice were injected intraperitoneally with 0.8×10⁶ cells/mouse with TCL1 #ST1 cells. The animals were assigned to 3 groups randomly on equitable basis of body weights.

Control group was injected with 20 □l of 0.05 mM phosphate buffered saline (PBS) pH 7.4 (none). PyNTTTGT group was injected with 20 □l of IMT504 (20 mg/ml) in PBS (total dose of 20 mg/kg animal weight) every day during the first 5 consecutive days. LLONC HA(1000)-ON1 group was injected with 20 □l of IMT504 (7.2 mg/ml) in PBS (3.5 mg/kg animal weight) in only one shot.

Eleven days after the treatment has started, peripheral blood samples were extracted, processed and quantified by mean of cytometric analysis, focused in the leukemic CD19+, CD5+ B cells (FIG. 29). Two days after that, mice were sacrificed and peritoneal cavity and spleen compartment were processed and leukemic cells analyzed by cytometry, measuring CD19+, CD5+ B cells (FIG. 29 right). Results were summarized in FIG. 29 (down left). Results shown demonstrated that HA(1000)-ON1 is much more potent drug than IMT504 for the treatment of CLL (at least around 30 times more, but much more considering that phosphorotioated IMT504 is around 30 times more active than phosphorodiester used in the LLONC HA(1000)-ON1 synthesis). The treatment with LLONCs could be a much better tool for treating this lymphoproliferative disorder, incurable and the most frequent type of leukemia in adult humans.

### Example 20: Effect of Myr-ONs in Chronic Lymphocytic Leukemia

WT mice were injected intraperitoneally with 0.8×10⁶ cells/mouse with TCL1 #ST1 cells. The animals were assigned to 4 groups randomly on equitable basis of body weights.

Control group was injected with 20 □l of 0.05 mM phosphate buffered saline (PBS) pH 7.4 (Control). PyNTTTGT group was injected with 20 □l of IMT504 (20 mg/ml) in PBS (total dose of 20 mg/kg animal weight) every day during the first 5 consecutive days. Myr-ON1 group was injected with 20 □l of Myr-ON1 in PBS (2 mg/kg animal weight) in only one shot. Myr-ON6 group was injected with 20 □l of Myr-ON6 in PBS (2 mg/kg animal weight) also in only one shot.

Five days after the treatment has started, mice were sacrificed and spleen compartment were processed and leukemic cells analyzed by cytometry, measuring CD19+, CD5+ B cells (FIG. 30 up). Results were summarized in FIG. 30 (down). Results shown demonstrated that Myr-ON6 is, at least, 10 times more potent drug than IMT504 for the treatment of CLL. On the other hand Myr-ON1 is, at least, 50 times more potent drug than IMT504 for the treatment of CLL, but much more considering that phosphorotioated IMT504 is around 30 times more active than phosphorodiester used in the Myr-ON1 synthesis). The treatment with LLONCs could be a much better tool for treating this lymphoproliferative disorder, incurable and the most frequent type of leukemia in adult humans.

### Example 21: Effect of PEG(40)-ONs in Chronic Lymphocytic Leukemia

WT mice were injected intraperitoneally with 0.8×10⁶ cells/mouse with TCL1 #ST1 cells. The animals were assigned to 4 groups randomly on equitable basis of body weights.

Control group was injected with 20 □l of 0.05 mM phosphate buffered saline (PBS) pH 7.4 (Control). PyNTTTGT group was injected with 20 □l of IMT504 (20 mg/ml) in PBS (total dose of 20 mg/kg animal weight) every day during the first 5 consecutive days. PEG(40)-ON1 group was injected with 20 □l of PEG(40)-ON1 in PBS (1 mg/kg animal weight) in only one shot. Myr-ON6 group was injected with 20 □l of PEG(40)-ON6 in PBS (1 mg/kg animal weight) also in only one shot.

Five days after the treatment has started, mice were sacrificed and spleen compartment were processed and leukemic cells analyzed by cytometry, measuring CD19+, CD5+ B cells (FIG. 31 Up). Results were summarized in FIG. 31 (down). Results shown demonstrated that PEG(40)-ON6 is, at least, 20 times more potent drug than IMT504 for the treatment of CLL. On the other hand PEG(40)-ON1 is, at least, 100 times more potent drug than IMT504 for the treatment of CLL, but much more considering that phosphorotioated IMT504 is around 30 times more active than phosphorodiester used in the PEG(40)-ON1 synthesis). The treatment with LLONCs could be a much better tool for treating this lymphoproliferative disorder, incurable and the most frequent type of leukemia in adult humans.

### Example 22: Effect of PEG(20)-CpG-ON and Palm-CpG-ON in Chronic Lymphocytic Leukemia

WT mice were injected intraperitoneally with 0.8×10⁶ cells/mouse with TCL1 #ST1 cells. The animals were assigned to 4 groups randomly on equitable basis of body weights.

Control group was injected with 20 □l of 0.05 mM phosphate buffered saline (PBS) pH 7.4 (Control). PyNTTTGT group (IMT504) was injected with 20 □l of IMT504 (20 mg/ml) in PBS (total dose of 20 mg/kg animal weight) every day during the first 5 consecutive days. CpG group (CpG2005) was injected with 20 □l of IMT504 (20 mg/ml) in PBS (total dose of 20 mg/kg animal weight) every day during the first 5 consecutive days. PEG(20)-ON5 group was injected with 20 □l of PEG(20)-ON5 in PBS (1 mg/kg animal weight) in only one shot. Palm-ON5 group was injected with 20 □l of Palm-ON5 in PBS (1 mg/kg animal weight) also in only one shot.

Five days after the treatment has started, mice were sacrificed and spleen compartment were processed and leukemic cells analyzed by cytometry, measuring CD19+, CD5+ B cells (FIG. 32 Up). Results were summarized in FIG. 31 (Down). Results shown demonstrated that PEG(20)-ON5 and Palm-ON1 are, at least, 100 times more potent drug for the treatment of CLL than the equivalent CpG2006, and even IMT504, but much more considering that phosphorotioated form is around 30 times more active than phosphorodiester used in the PEG(20)-ON5 and Palm-ON5 synthesis). The treatment with LLONCs could be a much better tool for treating this lymphoproliferative disorder, incurable and the most frequent type of leukemia in adult humans.

### Example 23: Effect of HA(1000)-ON1 in B16 F10 Melanoma Mouse Model

≤50% confluent monolayer B16-F10 melanoma cells in the logarithmic growth phase were detached with trypsin/EDTA and pipetted vigorously to obtain single-cell suspension. Then, the suspension is treated with cold culture medium to neutralize trypsin and centrifuged. Pelleted cells were resuspended in cold Hanks' balanced salt solution) HBSS, aiming for 1-5×10⁶ cells/ml, counted live cells using trypan blue (Viability >90%). Final cell concentration was adjusted to 1×10⁶ cells/ml in ice-cold HBSS. 100 μl of the cell suspension were inoculated to 6 to 12 weeks old female C57BL/6 mice into the tibialis anterior skeletal muscle and also subcutaneously into the right flank. Mice were observed for tumor growth; tumors became palpable in 5 to 10 days. When tumors reached ˜90 mm3 all treatments were initiated and the animals were assigned to 3 groups randomly on equitable basis of body weights.

Control group was injected with 20 □l of 0.05 mM phosphate buffered saline (PBS) pH 7.4 (none). PyNTTTGT group was injected with 20 □l of IMT504 (20 mg/ml) in PBS (total daily dose of 20 mg/kg animal weight) every day during the first 5 consecutive days. HA(1000)-ON1 group was injected with 3.5 mg/kg animal weight in only one shot.

Mice were observed for tumor growth and a caliper was used to measure perpendicular tumor diameters at time 9, 12, 14 and 16 days after the first (or only) shot of the treatment. The volume calculations were obtained using the formula V=(W (2)×L)/2 for caliper measurements, where L is the tumor length, W is the tumor width.

Results shown in the FIG. 33 demonstrated that HA(1000)-ON1 is much more potent drug than IMT504 for the treatment of melanoma tumors (at least around 30 times more, but much more considering that phosphorotioated IMT504 is around 30 times more active than phosphorodiester used in the LLONC synthesis.

### Example 24: Effect of HA(1000)-ON1 in 4T1 Breast Cancer Mouse Model

≤50% confluent monolayer 4T1 murine mammary carcinoma cells in the logarithmic growth phase were detached with collagenase and pipetted vigorously to obtain single-cell suspension. Then, the suspension is treated with cold culture medium to neutralize protease and centrifuged. Pelleted cells were resuspended in cold Hanks' balanced salt solution) HBSS, aiming for 1-5×10⁶ cells/ml, counted live cells using trypan blue (Viability >90%). Final cell concentration was adjusted to 7.5×10⁶ cells/ml in ice-cold HBSS. 100 μl of the cell suspension were subcutaneously injected to 6 to 12 week old female syngenic BALB/c mice into the inguinal flank area near to back of animals. After 6-8 days cancer were palpable in the cells injection position were observed for tumor growth. When tumors reached ˜90 mm3 all treatments were initiated and the animals were assigned to 3 groups randomly on equitable basis of body weights.

Control group was injected with 20 □l of 0.05 mM phosphate buffered saline (PBS) pH 7.4 (none). PyNTTTGT group was injected with 20 □l of IMT504 (20 mg/ml) in PBS (total dose of 20 mg/kg animal weight) every day during the first 5 consecutive days. HA(1000)-ON1 group was injected with 20 □l of IMT504 (7.2 mg/ml) in PBS (3.5 mg/kg animal weight) in only one shot.

Mice were observed for tumor growth and a caliper was used to measure perpendicular tumor diameters at time 8, 15, 22 and 29 days after the first (or only) shot of the treatment. The volume calculations were obtained using the formula V=(W (2)×L)/2 for caliper measurements, where L is the tumor length, W is the tumor width.

Results shown in the FIG. 34 demonstrated that HA(1000)-ON1 is much more potent drug than IMT504 for the treatment of this tumor, which very closely mimic stage IV human breast cancer. The HA(1000)-ON1 new molecule shown to be at least around 30 times more than PyNTTTTGT representative ODN, and much more considering that phosphorotioated IMT504 is around 30 times more active than phosphorodiester used in the LLONC synthesis).

### Example 25: Effect of HA(1000)-ON1 in Sepsis

18 mice were injected intraperitoneally with 30 □g/Kg of LPS.

Immediately, control group was injected with 25 □l of 0.05 mM phosphate buffered saline (PBS) pH 7.4 (none). IMT504 (PyNTTTGT group) was injected with 30 □g of IMT504 each every day during the first 5 consecutive days. HA(1000)-ON1 group A was injected with the equivalent of 60 □g of ODN in only one shot. HA(1000)-ON1 group B was injected with 15 □g of the equivalent of 60 □g of ODN in only one shot. HA(1000)-ON1 group C was injected with 6 □g of the equivalent of 60 □g of ODN in only one shot.

Animals were monitored periodically and survival ones in each group were counted. Results were expressed in a Kaplan Meier survival plot, shown in FIG. 35.

Results shown in the Fig demonstrated that HA(1000)-ON1 is much more potent drug than IMT504 for the treatment of sepsis (at least around 30 times more, but much more considering that phosphorotioated IMT504 is around 30 times more active than phosphorodiester used in the LLONC HA(1000)-ON1 synthesis). The treatment with HA(1000)-ON1 could be a much better drug for treating cytokine storm which causes sepsis.

### Example 26: Effect of HA-ONs on Cytokine Storm

13 mice were injected intraperitoneally with 20 □g/Kg of LPS. Another 6 with phosphate buffered saline (PBS) solution.

Immediately, control group was injected also with 20 □l of 0.05 mM phosphate buffered saline (PBS) pH 7.4 (Control). IMT504 (PyNTTTGT group) was injected with 600 □g of IMT504. HA(1000)-ON1 was injected with the equivalent of 70 □g of ODN in only one shot. Samples of serum were extracted at 2 and 24 h post treatment with LPS and serum levels of IL6 were measured.

Results shown in the FIG. 36 demonstrated that HA(1000)-ON1 is much more potent drug than IMT504 controlling the cytokine storm induced for LPS (at least around 10 times more, but much more considering that phosphorotioated IMT504 is around 30 times more active than phosphodiester used in the HA(1000)-ON1 synthesis). The treatment with LLONCs could be a much better drug for treating cytokine storm.

### Example 27: Effect of Palm-ON1 on Colitis Mouse Model

Colitis mouse model Male mice (6 weeks old) were randomly divided into five groups of 5 animals corresponding to control and treatments (5 mice per condition). On day 7, animals from Control group were intrarectally instilled with 100 μl of 50% ethanol (mucosal irritant and vehicle), and, all other groups were intrarectally instilled with 3.5% TNBS (an haptenic immunological stimulus) in 50% ethanol, PBS (vehicle).

Immediately, Control TNBS (−) group was injected (sc) with 200 □l of 0.05 mM phosphate buffered saline (PBS) pH 7.4; Control TNBS (+) group was injected (sc) with 200 □l of 0.05 mM phosphate buffered saline (PBS) pH 7.4. IMT504 group which was injected (sc) with 200 □l of IMT504 (2 mg/ml) in PBS (total dose of 2 mg/kg animal weight) every day during the first 5 consecutive days. Palm-ON1 200 group was injected (sc) with 200 □l of Palm-ON1 in PBS (200 g/kg animal weight) in only one shot and Palm-ON1 20 group which was injected (sc) with 200 □l of Palm-ON1 in PBS (20 g/kg animal weight) also in only one shot.

On day 14, animals were sacrificed, samples were taken, and the gut was removed. The colon was cut close to the ileocecal valve, and the length and weight were registered. Distal colon sections (2 cm) were cut and histologically analyzed as described in Muglia et al. (^{lxxx}). The histological activity index (HAI) was determined considering the magnitude of epithelial damage (score 0: none, 1: minimal loss of goblet cells, 2: extensive loss of goblet cells, 3: minimal loss of crypts, 4: extensive loss of crypts) and cellular infiltration (score 0: none infiltration, 1: infiltrate around crypt bases, 2: infiltrate in muscularis mucosa, 3: extensive infiltrate in muscularis mucosa with edema, 4: infiltration of submucosa). The results are shown in FIG. 37.

It is evident that all the treatments applied have been effective in controlling inflammatory colitis. It is also evident that the modified variant (Palm-ON1) is more powerful than IMT504 since a 500 times lower dose of LLONC generates better control of the pathology based on HAI. Even more so if we consider that we compare Palm-ON1 with its DNA phosphodiester component with fully phosphorothioate IMT504.

Application Project

| | |
|---|---|
| <1101> | Applicant name: Segena Corporation S.A |
| <120> | Title: Enhancing Oligonucleotide Immunomodulatory Activity through |
| | Dianophore Long-Lasting Modification: Methods and Applications |
| <130> | AppFileReference: |
| <140> | CurrentAppNumber: |
| <141> | CurrentFiling Date: |
| Sequence | |
| <213> | OrganismName: Artificial Sequence |
| <400> | PreSequenceString: |

| | |
|---|---|
| tttttttttt ttcattttgt gggg | 24 |

| | |
|---|---|
| <212> | Type: DNA |
| <211> | Length: 24 |
| | SequenceName: CpG 1 (Seq ID No 1) |
| | SequenceDescription: |
| Sequence | |
| <213> | OrganismName: Artificial Sequence |
| <400> | PreSequenceString: |

| | |
|---|---|
| tcatcatttt gtcattttgt catt | 24 |

| | |
|---|---|
| <212> | Type: DNA |
| <211> | Length: 24 |
| | SequenceName: PyNTTTTGT-1 (Seq ID No 2) |
| | SequenceDescription: |
| Sequence | |
| <213> | OrganismName: Artificial Sequence |
| <400> | PreSequenceString: |

| | |
|---|---|
| acatcatttt gtcattttgt catt | 24 |

| | |
|---|---|
| <212> | Type: DNA |
| <211> | Length: 24 |
| | SequenceName: PyNTTTTGT-2 (Seq ID No 3) |
| | SequenceDescription: |
| Sequence | |
| <213> | OrganismName: Artificial Sequence |
| <400> | PreSequenceString: |

| | |
|---|---|
| tttttttttt ttcattttgt gggg | 24 |

| | |
|---|---|
| <212> | Type: DNA |
| <211> | Length: 24 |
| | SequenceName: PyNTTTTGT-3 (Seq ID No 4) |
| | SequenceDescription: |
| Sequence | |
| <213> | OrganismName: Artificial Sequence |
| <400> | PreSequenceString: |

| | |
|---|---|
| acatcatttt gtcattttgt aatt | 24 |

| | |
|---|---|
| <212> | Type: DNA |
| <211> | Length: 24 |
| | SequenceName: PyNTTTTGT-4 (Seq ID No 5) |
| | SequenceDescription: |

ⁱ Gursel M, Verthelyi D, Gursel I, Ishii K J, Klinman D M. Differential and competitive activation of human immune cells by distinct classes of CpG oligodeoxynucleotides. J Leukoc Biol. 2002; 71(5): 813-820.
2 Elias F, Flo J, Lopez R. A., Zorzopulos J, Montaner A, Rodriguez J M (2003) Strong cytosine-guanosine-independent immunostimulation in humans and other primates by synthetic oligodeoxynucleotides with PyNTTTTGT motifs. J Immunol 171:3697-3704.
ⁱⁱⁱ Bode C, Zhao G, Steinhagen F, Kinjo T, Klinman D M. CpG DNA as a vaccine adjuvant. Expert Rev Vaccines. 2011; 10 (4): 499-511.
^{iv} Shirota H, Klinman D M. Recent progress concerning CpG DNA and its use as a vaccine adjuvant. Expert Rev Vaccines. 2014; 13(2): 299-312.
^{v} Krieg A M. CpG motifs in bacterial DNA and their immune effects. Annu Rev Immunol. 2002; 20:709-760.
^{vi} Gürsel M, Verthelyi D, Gürsel I, Ishii K J, Klinman D M. Differential and competitive activation of human immune cells by distinct classes of CpG oligonucleotide. J Leukoc Biol. 2002; 71(5): 813-820.
^{vii} Hartmann G, Wecratna R D, Ballas Z K, et al. Delineation of a CpG phosphorothioate oligodeoxynucleotide for activating primate immune responses in vitro and in vivo. J Immunol. 2000; 164(3): 1617-1624.
^{viii} Hartmann G, Krieg A M. Mechanism and function of a newly identified CpG DNA motif in human primary B cells. J Immunol. 2000; 164(2): 944-953.
^{ix} Zorzopulos J, Opal S M, Hernando-Insúa A, Rodriguez J M, Elías F, Fló J, López R A, Chasscing N A, Lux-Lantos V A, Coronel M F, Franco R, Montaner A D, Horn D L. Immunomodulatory oligonucleotide IMT504: Effects on mesenchymal stem cells as a first-in-class immunoprotective/immunoregenerative therapy.
World J Stem Cells. 2017 Mar. 26; 9(3): 45-67. doi: 10.4252/wjsc.v9.i3.45. Review.
^{x} Mutwiri G K, Nichani A K, Babiuk S, Babiuk L A. Strategies for enhancing the immunostimulatory effects of CpG oligodeoxynucleotides. J Control Release. 2004; 97 (1): 1-17. EP0912535B1
^{xii} Shechan J P, Lan H C. Phosphorothioate oligonucleotides inhibit the intrinsic tenase complex. Blood. 1998; 92(5): 1617-1625.
^{xiii} Henry S P, Novotny W, Leeds J, Auletta C, Kornbrust D J. Inhibition of coagulation by a phosphorothioate oligonucleotide. Antisense Nucleic Acid Drug Dev. 1997; 7(5): 503-510.
^{xiv} Shaw D R, Rustagi P K, Kandimalla E R, Manning A N, Jiang Z, Agrawal S. Effects of synthetic oligonucleotides on human complement and coagulation. Biochem Pharmacol. 1997; 53:1123-1132.
^{xv} Brown D A, Kang S H, Gryaznov S M, et al. Effect of phosphorothioate modification of oligodeoxynucleotides on specific protein binding. J Biol Chem. 1994; 269(43):26801-26805.
^{xvi} Henry S P, Beattie G, Ych G, et al. Complement activation is responsible for acute toxicit rhesus monkeys treated with a phosphorothioate oligodeoxynucleotide. Int Immunopharmacol. 2002; 2(12):1657-1666.
^{xvii} Bjersing J L, Eriksson K, Tarkowski A, Collins L V. The arthritogenic and immunostimulatory properties of phosphorothioate oligodeoxynucleotides rely on synergy between the activities of the nuclease-resistant backbone and CpG motifs. Inflammation. 2004; 28 (1): 39-51.
^{xviii} Heikenwalder M, Polymenidou M, Junt T, et al. Lymphoid follicle destruction and immunosuppression after repeated CpG oligodeoxynucleotide administration. Nat Med. 2004; 10(2): 187-192.
^{xix} Khvorova, Anastasia; Watts, Jonathan K (2017). The chemical evolution of oligonucleotide therapies of clinical utility. Nature Biotechnology, 35(3), 238-248. doi: 10.1038/nbt.3765. doi.org/10.1038/nbt.3765.
^{xx} WO 2007/085629 A2
^{xxi} US 2009/0197797
^{xxii} Schanté, C.E.; Zuber, G.; Herlin, C.; Vandamme, T.F. Chemical modifications of hyaluronic acid for the synthesis of derivatives for a broad range of biomedical applications. Carbohydr. Polym. 2011, 85, 469-489
^{xxiii} WO 2008/012365
^{xxiv} US 2009/0253651
^{xxv} WO 01/68105
^{xxvi} WO 2009/074678 A2
^{xxvii} Webster, R. and col. PEGylated proteins: evaluation of their safety in the absence of definitive metabolism studies Drug Metab. Dispos., 35 (2007), pp. 9-16
^{xxviii} Pasut, G. and col. State of the art in PEGylation: the great versatility achieved after forty years of research J. Control. Release, 161 (2012), pp. 461-472
^{xxix} Harris, J.M. and col. Effect of pegylation on pharmaceuticals Nat. Rev. Drug Discov., 2 (2003), pp. 214-221
^{xxx} Yamaoka, T. and col. Fate of water-soluble polymers administered via different routes J. Pharm. Sci., 84 (1995), pp. 349-354
^{xxxi} Ryan, Sinéad M and col. Advances in PEGylation of important biotech molecules: Delivery aspects Expert Opinion on Drug Delivery 2008, 5 (4): 371-83
^{xxxii} Xueguang Lu and Ke Zhang. PEGylation of therapeutic oligonucletides: From linear to highly branched PEG architectures. Nano Res. 2018 October; 11 (10): 5519-5534. doi: 10.1007/s12274-018-2131-8.
^{xxxiii} EP0912535B1 xxxiv EP0912535A1
^{xxxv} Zaro J L. Lipid-based drug carriers for prodrugs to enhance drug delivery. AAPS J. 2015 January; 17 (1): 83-92. doi: 10.1208/s12248-014-9670-z. Epub 2014 Oct. 1. PMID: 25269430; PMCID: PMC4287280.
^{xxxvi} Irby, Danielle; Du, Chengan; Li, Feng. Lipid-Drug Conjugate for Enhancing Drug Delivery. Mol. Pharmaceutics 2017, 14, 5, 1325-1338
^{xxxvii} IASP-pain.org [Internet]. International Association for the Study of Pain. IASP Taxonomy; 2012 May 22
^{xxxviii} Colloca L, Ludman T, Bouhassira D, Baron R, Dickenson A H, Yarnitsky D, Freeman R, Truini A, Attal N, Finnerup N B, Eccleston C, Kalso E, Bennett D L, Dworkin R H, Raja S N. Neuropathic pain. Nat Rev Dis Primers. 2017 Feb. 16; 3:17002. doi: 10.1038/nrdp.2017.2. PMID: 28205574; PMCID: PMC5371025.
^{xxxix} Lyon P, Cohen M, Quintner J. An evolutionary stress-response hypothesis for chronic widespread pain (fibromyalgia syndrome). Pain Med. 2011 August; 12(8):1167-78.
^{xl} Hargrove T. A. BetterMovement.org [Internet]. Systems Perspective on Chronic Pain; 2014 October 23 [cited 16 September 12].
^{xli} Cohen S P, Mao J. Neuropathic pain: mechanisms and their clinical implications. BMJ. 2014; 348: f7656. PubMed #24500412.
^{xlii} Clark I A. 2007. The advent of the cytokine storm. Immunol. Cell Biol. 85:271-273
^{xliii} Ferrara J L. 1993. Cytokine dysregulation as a mechanism of graft versus host disease. Curr. Opin. Immunol. 5:794-799
^{xliv} Link H. 1998. The cytokine storm in multiple sclerosis. Mult. Scler. 4:12-15
^{xlv} Makhija R, Kingsnorth A N. 2002. Cytokine storm in acute pancreatitis. J. Hepatobil. Pancreat. Surg. 9:401-410
^{xlvi} Wang H, Ma S. 2008. The cytokine storm and factors determining the sequence and severity of organ dysfunction in multiple organ dysfunction syndrome. Am. J. Emerg. Med. 26:711-715
^{xlvii} La Gruta N L, Kedzierska K, Stambas J, Doherty P C. 2007. A question of self-preservation: immunopathology in influenza virus infection. Immunol. Cell Biol. 85:85-92
^{xlviii} Imai Y, et al. 2008. Identification of oxidative stress and Toll-like receptor 4 signaling as a key pathway of acute lung injury. Cell 133:235-249
^{xlix} Rubenfeld G D, et al. 2005. Incidence and outcomes of acute lung injury. N. Engl. J. Med. 353:1685-1693
^{l} Pugin J, Ricou B, Steinberg K P, Suter P M, Martin T R. 1996. Proinflammatory activity in bronchoalveolar lavage fluids from patients with ARDS, a prominent role for interleukin-1. Am. J. Respir. Crit. Care Med. 153:1850-1856
^{li} Imai Y, et al. 2003. Injurious mechanical ventilation and end-organ epithelial cell apoptosis and organ dysfunction in experimental an model of acute respiratory distress syndrome. JAMA 289:2104-2112
^{lii} Simmons C, Farrar J. 2008. Insights into inflammation and influenza. N. Engl. J. Med. 359:1621-1623
^{liii} Mangione, Julia N.A. and col. The Association of Cytokines with Severe Dengue in Children. Trop Med Health. 2014 December; 42 (4): 137-144.
^{liv} Ward P.A. New approaches to the study of sepsis. EMBO Mol Med 4, 1234-1243 (2012).
^{lv} Richard S Hotchkiss, Guillaume Monneret, Didier Payen. Immunosuppression in sepsis: a novel understanding of the disorder and a new therapeutic approach. www.thelancet.com/infection Vol 13 March (2013).
^{lvi} Barochia A V, Cui X, Vitberg D, et al. Bundled care for septic shock: an analysis of clinical trials. Crit Care Med 38:668-78 (2010)
^{lvii} Monneret G, Venet F, Pachot A, Lepape A. Monitoring immune dysfunctions in the septic patient: a new skin for the old ceremony. Mol Med 14:64-78 (2008).
^{lviii} Kethireddy S, Kumar A. Mortality due to septic shock following early, appropriate antibiotic therapy: can we do better? Crit Care Med 40:2228-29 (2012).
^{lix} Otto G P, Sossdorf M, Claus R A, et al. The late phase of sepsis is characterized by an increased microbiological burden and death rate. Crit Care 15: R183 (2011).
lx J. Vollmer, A.M. Krieg/Advanced Drug Delivery Reviews 61 (2009) 195-204
^{lxi} Cheri D. Landers M D, Subbarao Bondada PhD What Insights Into Human Cord Blood Lymphocyte Function Can Be Gleaned From Studying Newborn Mice? in Hematology, Immunology and Infectious Disease, 2008. Mervin Yoder Ed.
^{lxii} Montaner A D, Denichilo A, Rodriguez J M, Flo J, Lopez R A, Pontoriero A, Savy V, Baumeister E, Frant R, Zorzopulos J, Elias F. 2011. Addition of the immunostimulatory oligonucleotide IMT504 to a seasonal flu vaccine increases hemagglutinin antibody titers in young adult and elder rats, and expands the anti-hemagglutinin antibody repertoire. Nucleic Acid Ther 21:265-274. http://dx.doi.org/10.1089/nat.2011.0284.
^{lxiii} Elias F, Flo J, Lopez R A, Zorzopulos J, Montaner A, Rodriguez J M. 2003. Strong cytosine-guanosine-independent immunostimulation in humans and other primates by synthetic oligodeoxynucleotides with PyNTTTTGT motifs. J Immunol 171:3697-3704. http://dx.doi.org/10.4049/jimmunol. 171.7.3697.
^{lxiv} Rodriguez J M, Elias F, Montaner A, Flo J, Lopez R A, Zorzopulos J, Franco R J, Lenial S P, Lopez Salón M, Pirpignani M L, Solimano J, Garay G, Riveros D, Fernandez J, Cacchione R, Dupont J. 2006. O N IMT504 induces an immunogenic phenotype and apoptosis of chronic lymphocytic leukemia cells. Medicina (B Aires) 66:9-16.
^{lx} Hernando Insua A, Montaner A D, Rodriguez J M, Elías F, Fló J, López R A, Zorzopulos J, Hofer E L, Chasseing N A. 2007. IMT504, the prototype of the immunostimulatory oligonucleotides of the PyNTTTTGT class, increases the number of progenitors of mesenchymal stem cells both in vitro and in vivo: potential use in tissue repair therapy. Stem Cells 25:1047-1054. http://dx.doi.org/10.1634/stemcells.2006-0479.
^{lxvi} Franco R, Rodriquez J M, Elias F, Hernando-Insua A, Flo J, Lopez R, Nagle L, Zorzopulos J, Horn D L, Montaner A D. 10 Apr. 2014, posting date. Non-clinical safety studies of IMT504, a unique non-CpG oligonucleotide. Nucleic Acid Ther http://dx.doi.org/10.1089/nat.2013.0479.
^{lxvii} Hernando-Insúa A, Rodriguez J M, Elías F, Fló J, López R, Franco R, Lago N, Zorzopulos J, Montaner A D. 2010. A high dose of IMT504, the PyTTTTGT prototype immunostimulatory oligonucleotide, does not alter embryonic development in rats. Oligonucleotides 20:33-36. http://dx.doi.org/10.1089/oli.2009.0206.
^{lxviii} Rodríguez J M, Elias F, Montaner A et al. Oligonucleotide IMT504 induces an immunogenic phenotype and apoptosis in chronic lymphocytic leukemia cells. Medicina (B Aires) 2006; 66:9-16.
^{lxix} Andrés Hernando Insua, Alejandro D. Montaner, Juan M. Rodriguez, Fernanda Elías, Juan Fló, Ricardo A. López, Jorge Zorzopulos, Erica L. Hofer and Norma A. Chasseing. IMT504, the Prototype of the Immunostimulatory Oligonucleotides of the PyNTTTTGT Class, Increases the Number of Progenitors of Mesenchymal Stem Cells Both In Vitro and In Vivo: Potential Use in Tissue Repair Therapy. Stem Cells 2007; 25; 1047-1054. DOI: 10.1634/stemcells.2006-0479
^{lxx} Jorge Zorzopulos, Steven M Opal, Andrés Hernando-Insúa, Juan M Rodriguez, Fernanda Elías, Juan Fló, Ricardo A López, Norma A Chasseing, Victoria A Lux-Lantos, Maria F Coronel, Raul Franco, Alejandro D Montaner, David L Horn. Immunomodulatory oligonucleotide IMT504: Effects on mesenchymal stem cells as a first-in-class immunoprotective/immunoregenerative therapy. World J Stem Cells 2017 Mar. 26; 9(3):45-67. DOI: 10.4252/wjsc.v9.13.45
^{lxxi} De Mesmacker et al. Acc. Chem. Res. 1995, 28:366-374
^{lxxii} Sumnierton and Weller, U.S. Pat. No. 5,034,506
^{lxxiii} Nielsen etal. Science 1991, 254, 1497
^{lxxiv} U.S. Pat. No. 6,468,798
^{lxxv} U.S. Pat. No. 4,522,811
^{lxxvi} Hargreaves K M, Dubner R, Brown F, Flores C, Joris J. A new and sensitive method for measuring thermal nociception in cutaneous hyperalgesia. PAIN 1988; 32:77-88.
^{lxxvii} Chaplan S R, Bach F W, Pogrel J, Chung J M, Yaksh T L. Quantitative assessment of tactile allodynia in the rat paw. J Neurosci Methods 1994; 53:55-63.
^{lxxviii} Dixon W J. Efficient analysis of experimental observations. Annu Rev Pharmacol Toxicol 1980; 20:441-62.
^{lxxix} Galanski, M.; Jakupec, M. A.; Keppler, B. K. Update of the preclinical situation of anticancer platinum complexes: Novel design strategies and innovative analytical approaches. Curr. Med. Chem. 2005, 12, 2075-2094.
^{lxxx} Muglia, C. I., Papa Gobbi, R., Smaldini, P., Orsini Delgado, M. L., Candia, M., Zanuzzi, C., Sambuelli, A., Rocca, A., Toscano, M. A., Rabinovich, G. A., Docena, G. H., 2016. Inflammation controls sensitivity of human and mouse intestinal epithelial cells to galectin-1. J. Cell. Physiol. 231, 1575-1585.

1. A covalent modification of a dianophore structure of an oligonucleotide, comprising:
a long-lasting agent conjugated to an immunomodulatory oligonucleotide component,
wherein the immunomodulatory oligonucleotide component is covalently linked through a spacer containing at least one active group covalently linked to the dianophore modifier.

2. (canceled)

3. The covalent modification according to claim 1, wherein the long-lasting agent is:
a hyaluronic acid;
a hyaluronic acid having a Mw of about 15 to 1000 kDaltons;
a covalently linked through the C6 on the glucuronic acid subunit of the hyaluronic acid.

4. (canceled)

5. (canceled)

6. (canceled)

7. (canceled)

8. A method for producing the covalent modification to claim 3, wherein the method comprises the steps of:
covalent coupling a carboxylic group of a glucuronic acid subunit of the hyaluronic acid and an amino group of a spacer on a 3′ end of the immunomodulatory oligonucleotide component by with a condensing agent selected from the group consisting of a soluble monocarbodiimide and a soluble biscarbodiimide;
covalent coupling a carboxylic group of a glucuronic acid subunit of the hyaluronic acid and an amino group of a spacer in any other position than a 3′ end of a backbone of the immunomodulatory oligonucleotide component by with condensing agent selected from the group consisting of soluble monocarbodiimide or a soluble biscarbodiimide;
covalent coupling an acyl chloride group of the polyethylene glycol derivative and the amino group on the 3′ end of the immunomodulatory oligonucleotide component;
covalent coupling an anhydride group of the pol ethylene glycol derivative and an amino group on the 3′ end of the immunomodulatory oligonucleotide component;
covalent coupling a thiocyanate group of the polyethylene glycol derivative and an amino group on a 3′ end of the immunomodulatory oligonucleotide component; or
covalent coupling a thiocyanate group of the polyethylene glycol derivative and an amino group of a spacer in any other position other than a 3′ end of a backbone of the immunomodulatory oligonucleotide component.

9. (canceled)

10. The covalent modification according to claim 1, wherein the long-lasting agent is:
a polyethylene glycol derivative in which one of a terminal-OH group is transformed into a carboxylic group, an acyl chloride group, an aldehyde, an anhydride or an isothiocyanate group; or
a polyethylene derivative ha vino a terminal carboxylic acid and terminal groups including a malonic acid, a glutaric acid, an adipic acid, a pimelic acid, or a fumaric acid.

11. (canceled)

12. (canceled)

13. (canceled)

14. (canceled)

15. (canceled)

16. (canceled)

17. (canceled)

18. (canceled)

19. (canceled)

20. (canceled)

21. The covalent modification according to claim 1, where the long-lasting agent conjugated to the dianophore of a biologically active oligonucleotide is:
a lipid;
palmitic acid; or
a myristic acid;
wherein the carboxylic group of the lipidic acid is condensed with the amino group on 3′ end of the oligonucleotide.

22. (canceled)

23. (canceled)

24. (canceled)

25. (canceled)

26. (canceled)

27. The covalent modification according to claim 1 wherein the immunomodulatory oligonucleotide component has a length from 22 to 70 nucleotides, which encompasses the sequence motif represented by the following sequence:
(SEQ ID No: 1)
5′-TCGTCGTTTTGTCGTTTTGTCGTT-3′;
wherein the covalent modification includes:
at least a 90% sequence identity to SEQ ID NO: 1 and at least one unmethylated CpG pair;
at least a 80% sequence identity to SEQ ID NO: 1 and at least one unmethylated CpG pair; or
at least a 50% sequence identity to SEQ ID NO: 1 and at least one unmethylated CpG pair.

28. (canceled)

29. (canceled)

30. (canceled)

31. (canceled)

32. (canceled)

33. (canceled)

34. The covalent modification according to claim 10, wherein the immunomodulatory oligonucleotide component is:
covalently linked through a modified deoxyribose at 3′ end;
covalently linked through a modified sugar located near to the 3′ end of the ON chain;
a modified sugar located anywhere in the ON chain;
covalently linked through a spacer containing at least one active amino group;
covalently linked through a spacer comprising a linear hydrocarbon chain having about 2 to 14 C;
covalently linked through a spacer comprising a branched hydrocarbon chain having about 3 to 40 C;
covalently linked through a spacer with an active amine group; or
covalently linked through a spacer with an active sulfhydryl group.

35. (canceled)

36. (canceled)

37. (canceled)

38. The covalent modification according to claim 1, wherein the immunomodulatory oligonucleotide component has a length from 22 to 70 nucleotides, which encompasses the sequence motif represented by the following sequence:
(SEQ ID No: 2)
5′-TCATCATTTTGTCATTTTGTCATT-3′,
wherein the covalent modification includes:
at least 90% sequence identity to SEQ ID NO: 2. In all cases, the sequence should conserve at least one active site with the sequence PyNTTTTGT;
at least 80% sequence identity to SEQ ID NO: 2. In all cases, the sequence should conserve at least one active site with the sequence PyNTTTTGT; or
at least 50% sequence identity to SEQ ID NO: 2. In all cases, the sequence should conserve at least one active site with the sequence PyNTTTTGT.

39. (canceled)

40. (canceled)

41. (canceled)

42. The covalent modification according to claim 38, wherein the backbone of the immunomodulatory oligonucleotide component is:
fully or partially phosphorotioated;
total or partial nucleosyl amino acid-modification; or
has a total or a partial modification increasing its resistance to nucleases degradation.

43. (canceled)

44. (canceled)

45. The covalent modification according to claim 38, wherein the immunomodulatory oligonucleotide component is:
covalently linked through a modified deoxyribose at 3′ end;
covalently linked through a modified sugar located near to the 3′ end of the ON chain;
covalently linked through a modified sugar located anywhere in the ON chain;
covalently linked through a spacer containing at least, one active amino group;
covalently linked through a spacer comprising a linear hydrocarbon chain having about 2 to 14 C;
covalently linked through a spacer comprising a branched hydrocarbon chain having about 3 to 40 C;
covalently linked through a spacer with an active amine group; or
covalently linked through a spacer with an active sulfhydryl gorup.

46. (canceled)

47. (canceled)

48. (canceled)

49. (canceled)

50. (canceled)

51. (canceled)

52. (canceled)

53. A method for preventing or treating primary or metastatic cancer, sepsis, a cytokine storm syndrome caused by a viral infection, pain or precluding the pain chronification, a neurodegenerative disease, an inflammatory disease, immunodeficiency disorders, autoimmune diseases, aging conditions, intestinal irritation, reduction of secondary effects due to cytostatic during cancer treatment, any disorder, the method comprising the step off administering to a patient in need of a therapeutically effective amount of the covalent modification according to claim 1 or a pharmaceutically acceptable preparation thereof, alone or administered prior to and/or simultaneously with and/or after initiation of one or more.

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. A method for coating an implantable device with a physiologically active amount of the covalent modification according to claim 1 and an effective amount a bioactive agent.

66. A method according to claim 53, wherein the covalent modification according is administered intranasally, transdermally, orally, (liquid, capsules, tablets, or chewable tablets), sublingual, buccal, rectal, vaginal, ocular, otic or injection (subcutaneous, intramuscular, intravenous, intraperitoneal, intracardiac, intraarticular, and intracavernous) routes, continuous infusion (intravenous or intraperitoneal), or locally administered to a tissue or organ.

67. A method according to claim 53, wherein the covalent modification is administered in a dose of about 1 microgram to 10 milligram per Kg of body mass.

68. (canceled)

69. (canceled)

70. (canceled)

71. (canceled)

72. (canceled)

73. The covalent modification according to claim 1, wherein the long-lasting agent is:
a polyethylene glycol;
a polyethylene glycol having a Mw of about 15 to 100 kDaltons;
a linear polyethylene glycol; or
a branched polyethylene glycol.


SEQUENCE LISTING (5 sequences)

SEQ ID NO: 1 - DNA, 24 bp, synthetic construct
  source 1..24: mol_type = other DNA
ttttttttttttcattttgtgggg

SEQ ID NO: 2 - DNA, 24 bp, synthetic construct
  source 1..24: mol_type = other DNA
tcatcattttgtcattttgtcatt

SEQ ID NO: 3 - DNA, 24 bp, synthetic construct
  source 1..24: mol_type = other DNA
acatcattttgtcattttgtcatt

SEQ ID NO: 4 - DNA, 24 bp, synthetic construct
  source 1..24: mol_type = other DNA
ttttttttttttcattttgtgggg

SEQ ID NO: 5 - DNA, 24 bp, synthetic construct
  source 1..24: mol_type = other DNA
acatcattttgtcattttgtaatt